SEQUENCE LISTING (5 sequences)

SEQ ID NO: 1 - protein, 20 aa, homo sapien
ALCRRSTTDCGGPKDHPLTS

SEQ ID NO: 2 - protein, 15 aa, homo sapien
SYAVALSCQCALCRR

SEQ ID NO: 3 - protein, 15 aa, homo sapien
SLEPLRPRCRPINAT

SEQ ID NO: 4 - protein, 15 aa, homo sapien
SYAVALSAQCALCRR

SEQ ID NO: 5 - protein, 15 aa, homo sapien
SFPVALSCRCGPCRR

(19) United States
(12) Patent Application Publication
(10) Pub. No.: US 2024/0000895 A1
(43) Pub. Date: Jan. 4, 2024

(54) Treatment Of Females Having BRCA1/2 Mutations With Human Chorionic Gonadotropin To Reduce The Risk Of Developing Breast Cancer

(71) Applicants: Institute For Cancer Research d/b/a The Research Institute of Fox Chase Cancer Center, Philadelphia, PA (US); Universiteit Gent, Ghent (BE)

(72) Inventors: Jose Russo, Philadelphia, PA (US); Herman Depypere, Ghent (BE); Yanrong Su, Philadelphia, PA (US); Nhi Dang, Philadelphia, PA (US); Jaak Janssens, Ghent (BE)

(21) Appl. No.: 18/265,113

(22) PCT Filed: Dec. 6, 2021
(86) PCT No.: PCT/US21/61956

§ 371 Date: Jun. 2, 2023

Related U.S. Application Data
(60) Provisional application No. 63/122,023, filed on Dec. 7, 2020.

(51) Int. Cl. A61K 38/24; A61P 35/00; G01N 33/574; A61K 45/06

(52) U.S. Cl. CPC A61K 38/24; A61P 35/00; G01N 33/57415; A61K 45/06

(57) ABSTRACT

Methods of treating nulligravid females having a high risk of developing breast cancer and without exposure to a contraceptive by administering human chorionic gonadotropin (hCG), methods of monitoring the treatment efficacy of a subject having breast cancer or having a high risk of developing breast cancer, and methods of determining whether a subject is at risk of developing breast cancer are provided herein.

### REFERENCE TO SEQUENCE LISTING

This application includes a Sequence Listing filed electronically as a text file named 18530009502SEQ, created on Dec. 4, 2021, with a size of 2 kilobytes. The Sequence Listing is incorporated herein by reference.

### FIELD

The present disclosure is directed, in part, to treating nulligravid females having a high risk of developing breast cancer and without exposure to a contraceptive by administering human chorionic gonadotropin (hCG), methods of monitoring the treatment efficacy of a subject having breast cancer or having a high risk of developing breast cancer, and methods of determining whether a subject is at risk of developing breast cancer.

### BACKGROUND

Breast cancer is estimated to be the leading cause of death in women age 35 to 54 and accounts for 27% of all malignancies worldwide. One of the established risk factors for breast cancer is a BRCA1 and BRCA2 germ line mutations, that confer a lifetime risk of up to 70%. Carriers of these mutations therefore constitute a cohort with the highest risk. Breast cancer prevention in these women is challenging. To date, bilateral mastectomy remains the most effective means of reducing the incidence of BRCA-associated breast cancer. Chemoprevention with selective estrogen receptor modulators such as tamoxifen and aromatase inhibitors have been used to reduce breast cancer development for women at high risk, but it has not been validated as a chemopreventive method for primary breast cancer in BRCA1 mutation carriers.

Although there is an association between early full term pregnancy and a reduction in the lifetime risk of developing breast cancer, the mechanism providing this protection is still being determined. hCG represents one of the four members of the glycoprotein family which also include follitropin (FSH), thyrotropin (TSH), and lutropin (LH). hCG is a heterodimeric consisting of a 92 amino acid α (alpha) subunit and a 145 amino acid β (beta) subunit. The a subunit is ubiquitous among the four glycoprotein families while the β subunit is limited to hCG. While hCG is typically produced by syncytiotrophoblasts in the placenta after implantation, it is also upregulated in certain cancer tumors in both males and females. In particular, overexpression leading to β subunit secretion in various cancer cell types has been observed independent of α subunit gene expression.

### SUMMARY

The present disclosure provides methods of treating a nulligravid female having a high risk of developing breast cancer, the methods comprising administering hCG two to four times a week for at least ten weeks, wherein the nulligravid female is without exposure to a contraceptive for at least 21 days prior to administration of the hCG, thereby reducing the risk of developing breast cancer.

The present disclosure also provides methods of monitoring the efficacy of treatment of a subject having breast cancer or having a high risk of developing breast cancer, the methods comprising: a) obtaining or having obtained a biological sample from the subject prior to treatment initiation (T1) to provide a baseline expression of a panel of genes from the biological sample; b) obtaining or having obtained a biological sample from the subject after treatment completion (T2); and c) obtaining or having obtained a biological sample from the subject about 6 months or later after treatment completion (T3); and d) performing a gene expression assay on the T1, T2, and T3 samples to identify a set of differentially expressed genes from the biological sample; wherein increased expression in at least 10 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 5 of the following genes: FBL, FZD1, FZD7, HMAG1, ITGB4, KIT, LIG1, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample at T3 compared to T1 indicates that the treatment is efficacious, and wherein a lack of the differentially expressed gene profile indicates that the treatment is non-efficacious.

The present disclosure also provides methods of monitoring the efficacy of treatment of a subject having breast cancer or having a high risk of developing breast cancer, the methods comprising: a) obtaining or having obtained a biological sample from the subject prior to treatment initiation (T1) to provide a baseline expression of a panel of genes from the biological sample; b) obtaining or having obtained a biological sample from the subject after treatment initiation (T1); and c) performing a gene expression assay on the two samples to identify a set of differentially expressed genes from the biological sample; wherein increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

The present disclosure also provides methods of determining whether a subject is at risk of developing breast cancer, the method comprising obtaining or having obtained a biological sample from the subject and performing a gene expression assay to identify an expression profile of a panel of genes from the biological sample; wherein increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, KIT, ID4, miR182, MMP7, MYC, PADI2, SOX9, and SOX10, in the biological sample obtained from the subject compared to a control breast cancer expression profile indicates that the subject has a lower risk of developing breast cancer; and when the subject does not have the expression profile, the subject is at higher risk of developing breast cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a flow chart of participant recruitment and sample size used for each step of the study.

FIG. 2, Panel A shows the size of one breast biopsy specimen; FIG. 2, Panel B shows appropriately preserved tissue morphology; the lobules and ducts were clearly identified by H&E staining, and the nuclear structure was also preserved in these cells (magnification: 200×); FIG. 2, Panel C shows breast epithelial cells in lobules and ducts stained positive for E-cadherin on the cell membrane and cytoplasm, with a more intense staining on the cell membrane (magnification: 200×); FIG. 2, Panel D shows staining of H3K27me3 located on the cell nuclei (magnification: 400×).

FIG. 3 shows DEGs at the cutoff fold change (FC) of 1.5 and 2, respectively, with 1907 DEGs (1032 up, 875 down) at T2 vs. T1 and 1065 DEGs (897 up, 168 down) at T3 vs. T1 for the women not using contraceptives (responders) while there was almost no response at T2 vs. T1 and a small number of DEGs, 260 (214 up, 46 down) at T3 vs. T1 for the group of 14 women using pills or an IUD during, or stopping the pills prior to, the trial (low responders); the graphs represent the number of DEGs found in the breast tissue of women at different time points after hCG treatment compared to the control samples taken from the same patient before treatment; cutoff of false discovery rate-adjusted p-value (FDRp)<0.05 and Fold change of 1.5 or 2. Bar clusters=down-regulated (left bar); up-regulated (right bar).

FIG. 4, Panel A shows mean ovary 2-dimensional size and ovary thickness with 95% confidence interval; FIG. 4, Panel B shows mean ovary 2-dimensional size and endometrial thickness with 95% confidence interval.

FIG. 5, Panel A shows responders had the lowest level at week 5 and the peak (or close to peak) at week 36 for both serum FSH; mean FSH and LH with 95% confidence intervals according to visit and response; mean FSH level at weeks 5, 9, and 13 in low responders is 3.26 (2.15-4.95) and is significantly (p=0.028) higher the mean value 1.64 (1.05-2.56) of the responders at week 5, 9, and 13; FIG. 5, Panel B shows responders had the lowest level at week 5 and the peak (or close to peak) at week 36 for both serum LH; mean FSH and LH with 95% confidence intervals according to visit and response; mean LH level at weeks 5, 9, and 13 of the low responders was not different from the responders (p=0.204).

FIG. 6, Panel A shows a higher level of estradiol (p=0.078, mean ratio=0.55) compared to low responders at week 1; mean estradiol with 95% confidence interval according to visit and response; FIG. 6, Panel B shows a higher level of progesterone (p=0.01, mean ratio=0.2) compared to low responders at week 1; mean progesterone with 95% confidence interval according to visit and response.

FIG. 7, Panel A shows mean hCG level was 206 (180-237) IU/L at week 5, 9, and 13 in the low responders, it was significantly (P<0.005) higher than the mean value 154 (134-178) IU/L in the responders (mean ratio low responders to responders=1.34); mean hCG with 95% confidence intervals according to visit and response; FIG. 7, Panel B shows levels of prolactin were not significantly different between groups, not even when they were pooled; mean hCG with 95% confidence intervals according to visit and response.

FIG. 8 shows there is also no DEGs related to DNA repair at both T2 and T3, and DEGs associated with chromatin remodeling and organization (6 DEGs) as well as cell cycle (3 DEGs); rhCG effects on DNA repair, chromatin remodeling-organization and cell cycle are abolished by the hormonal birth control exposure in breasts of women carrying BRCA1/2 mutation; the graph represents the number of differentially expressed genes found in the breast tissues of women for each categorized genomic function at different time point compared to the control samples taken from the same patient before treatment. Bar clusters=without contraceptives (left bar); with contraceptives (right bar).

FIG. 9, Panel A shows BRCA1 protein was significantly higher in the breast tissues of BRCA1/2 wild type women; breast biopsy of BRCA1/2 wild type or mutation carrier was used for IHC staining; paraffin sections at 4 μm were stained with BRCA1-N antibody; the analysis based on the intensity; FIG. 9, Panel B shows representative IHC images of BRCA1 or BRCA2 mutation carrier without contraceptives use (magnification, 400×; scale bar, 20 μm).

FIG. 10 shows representative IHC images for subjects without contraceptives; the effect of rhCG treatment on H3K27me3 in breast epithelial cells of BRCA1/2 carriers; representative images of H3K27me3 staining in breast tissues of BRCA1/2 mutation carriers without contraceptives use (magnification, 400×; scale bar, 20 μm).

FIG. 11, Panel A shows that 50 IU/ml of rhCG treatment induced up-regulation of BRCA1 and BARD1 in MCF10F cells at the end of treatment and persisted 5-days post treatment stopped; MCF10F cells were treated with 10 or 50 IU/ml of rhCG for 72 hours, total lysates were extracted at the end of treatment or 5 days post rhCG treatment, 40 μg protein were used for WB; MCF10A or MCF12A cells were treated with 50 IU/ml rhCG for 72 hours, nuclear fraction was extracted at the end of treatment, 30 μg protein was used for WB; the number under each band indicates the relative expression quantified by intensity of the band; FIG. 11, Panel B, Panel C, and Panel D show MCF10A cells treated with 50 IU/ml rhCG for 72 hours, nuclear fraction was extracted at the end of treatment, and 6 days as well as 10 days post treatment, 30 μg protein was used for WB; the intensity of each band was quantified and graphed. Bar clusters=BRCA1^{+/+}, Ctrl (first bar); BRCA1^{+/+}, hCG (second bar); BRCA1^{mut/+}, Ctrl (third bar); BRCA1^{mut/+}, hCG (fourth bar).

FIG. 12, Panel A shows the protein level of TGFβ was increased in BRCA1^{+/+} cells at the end of 72 hours rhCG treatment; MCF10A cells were treated with 50 IU/ml rhCG for 72 hours, total lysates or nuclear fraction was extracted at the end of treatment, and 6-days as well as 10-days post treatment, 30 μg protein was used for WB; TGFβ and SFRP4 were analyzed by using total lysates, and SOX7 was analyzed by using nuclear fraction; FIG. 12, Panel B shows the expression level of miR182 in both BRCA1^{+/+} and BRCA1^{mut/+} cell lines with and without rhCG treatment; FIG. 12, Panel C shows the quantification of SFRP4, TGF beta, and SOX7 in both BRCA1^{+/+} and BRCA1^{mut/+} cell lines with and without rhCG treatment. Bar clusters=BRCA1^{+/+}, Ctrl (first bar); BRCA1^{+/+}, hCG (second bar); BRCA1^{mut/+}, Ctrl (third bar); BRCA1^{mut/+}, hCG (fourth bar).

FIG. 13, Panel A shows p53 protein was increased in both BRCA1 WT and mutation carrier MCF10A cells at 6 days and 10 days post rhCG treatment detected by WB; BRCA1 wild type or mutation carrier MCF10A cells were treated with 50 IU/ml of rhCG for 72 hours, nuclear fraction was extracted at the end of treatment and 6-days as well as 10-days post rhCG treatment. 30 μg protein was used for WB; FIG. 13, Panel B shows p53 protein was increased in both BRCA1 WT and mutation carrier MCF10A cells at 6 days and 10 days post rhCG treatment detected by WB; the intensity of each band in A was quantified and graphed. Bar clusters=BRCA1^{+/+}, Ctrl (first bar); BRCA1^{+/+}, hCG (second bar); BRCA1^{mut/+}, Ctrl (third bar); BRCA1^{mut/+}, hCG (fourth bar); FIG. 13, Panel C shows immunofluorescence staining also detected the increase of p53 at the end of 72 hours treatment; representative images of cells stained with p53 by immunofluorescence (magnification, 400×), at the end of 72-hours treatment.

FIG. 14, Panel A shows the gamma H2AX level at 24 hours post gamma irradiation was decreased by 56% when cells were treated with rhCG before irradiation, although the gamma H2AX level was same at 1-hour post irradiation; this effect was also observed 5 days post rhCG treatment; MCF10F cells were treated with 50 IU/ml rhCG for 72 hours, then cells were irradiated with 2 Gy gamma irradiation (IR) at the end of rhCG treatment, or irradiated at 5 days post rhCG treatment; gamma H2AX level was evaluated by WB at indicated time points post IR; FIG. 14, Panel B shows decreased gamma H2AX level in total cell lysates of rhCG treated cells evaluated by WB, reduced number of gamma H2AX foci on the nuclei of cells treated with rhCG was observed by immunofluorescence staining of gamma H2AX; representative images and quantification of gamma H2AX foci in MCF 10F cells, cells were irradiated after 72 hours rhCG treatment; FIG. 14, Panel C shows decreased gamma H2AX level in total cell lysates of rhCG treated cells evaluated by WB, reduced number of gamma H2AX foci on the nuclei of cells treated with rhCG was observed by immunofluorescence staining of gamma H2AX; representative images and quantification of gamma H2AX foci in MCF 10F cells, cells were irradiated after 72 hours rhCG treatment; FIG. 14, Panel D shows decreased gamma H2AX level when compared with cells without rhCG treatment prior to gamma irradiation (MCF10A cells with BRCA1^{+/+} or BRCA1^{mut/+} were treated with 50 IU/ml rhCG for 72 hours, then cells were irradiated with 2 Gy gamma irradiation at the end of rhCG treatment, or 5 Gy gamma irradiation at 9-days post rhCG treatment; gamma H2AX level was evaluated by WB at indicated time points post IR; the number below the WB band indicates the relative expression level to the control; FIG. 14, Panel E shows increased DNA repair compared to cells without rhCG treatment; quantification of gamma H2AX foci in MCF10A cells; cells were treated with 50 IU/ml of rhCG for 72 hours first, then at 9-days post rhCG treatment, cells were irradiated with 5 Gy gamma irradiation, foci were quantified 6 hours post irradiation; * indicates p<0.05 by Chi-Square analysis.

FIG. 15, Panel A shows H3K27me3 level in the rat mammary gland epithelial cells by immunohistochemistry, the global H3K27me3 level and the number of cells positive for H3K27me3 was increased in rat mammary gland 15-days post rhCG treatment, at a level similar to that in the mammary gland of 15 days post-delivery; Sprague Dawley rats were treated with 100 IU/day rhCG for 21 days or mated at 55 days old; mammary glands were collected 15-days post treatment or delivery; IHC to H3K27me3 antibody was performed on paraffin sections; * indicates p<0.05 compared to control (magnification, 400×); FIG. 15, Panel B shows H3K27me3 was increased in both BRCA1^{+/+} or BRCA1^{mut/+} cells, at the time of finishing 72 hours rhCG treatment, and 6 days or 10-days post rhCG treatment; MCF10A cells were treated with 50 IU/ml rhCG for 72 hours, nuclear fraction was extracted at the end of treatment, or 6-days and 10-days post rhCG treatment; 30 μg protein was used to perform WB; FIG. 15, Panel C shows H3K27me3 was increased in both BRCA1^{+/+} or BRCA1^{mut/+} cells, at the time of finishing 72 hours rhCG treatment, and 6 days or 10-days post rhCG treatment; quantification of WB was shown. Bar clusters=BRCA1^{+/+}, Ctrl (first bar); BRCA1^{+/+}, hCG (second bar); BRCA1^{mut/+}, Ctrl (third bar); BRCA1^{mut/+}, hCG (fourth bar); FIG. 15, Panel D shows H3K27me3 was increased in both BRCA1^{+/+} or BRCA1^{mut/+} cells, at the time of finishing 72 hours rhCG treatment, and 6 days or 10-days post rhCG treatment; MCF10A cells were plated in 4-well chamber slides, cells were treated with 50 IU/ml rhCG for 72 hours, then fixed and stained with H3K27me3 by immunofluorescence staining; representative image is shown (magnification, 400×).

FIG. 16, Panel A shows a number of primary mammospheres generated from mammary epithelial cells of rats 21-days post rhCG treatment was significantly reduced when compared with that from control rats (56.6±4.0 mammospheres for control, 37.2±2.0 for rhCG group, n=3, t-test p=0.002); representative images of mammospheres; epithelial cells enriched rat mammary cells were plated on ultra-low attachment 6-well plate at a density of 25,000 cells/ml in complete EpiCult-B medium; the mammospheres formation was checked daily and took pictures every other day; FIG. 16, Panel B shows Cd24 and CD10 are both significantly down-regulated by Microarray and RT-PCR analysis; validation of selected genes by real-time RT-PCR; RNAs extracted from primary mammopsheres were used for microarray and PCR analysis. Bar clusters=Real-time RT PCR (left bar); microarray (right bar); FIG. 16, Panel C shows expression of CK14 is significantly increased in mammospheres from rhCG treated rats (35±3.5% of mammospheres are positive in hCG group whereas only 18±3.8% are positive for control, T test p=0.0088) (validation the expression of CK14 in primary mammospheres by IHC analysis); FIG. 16, Panel D shows IHC staining of rat mammary gland also showed cd24 was reduced significantly (p=0.05) in mammary epithelial cells of rhCG treated rats (validation the expression of cd24 in rat mammary glands; the formalin fixed paraffin embedded rat mammary glands were used for IHC analysis, rats are from the same group of animals those were used to isolate mammary epithelial cells for mammosphere culture.

FIG. 17, Panel A shows Venn diagrams representing the number of DEGs found up and down-regulated (FC≥1.5) at T2 and T3 compared to the control sample of the same patient at T1, and the common DEGs between 2 time points; sample size: n=11 women for responders, n=14 women for low-responders; Panel B shows Venn diagrams representing the number of differentially expressed genes (DEGs) found up and down-regulated (FC≥2) in the breast tissue of BRCA1/2 carriers at T2 and T3 compared to the control sample taken from the same patient before treatment (T1); sample size: n=11 individuals for responders, n=14 individuals for low-responders; notably, in the responders, the number of DEGs with cutoff FC2 (1327 DEGs) accounts for almost half of the total number of DEGs (2972 DEGs) with cutoff FC1.5; Panel C shows Volcano plots of pairwise comparisons for DEGs with FC≥1.5; the y-axis is the negative log 10 of FDR-adjusted p values (−log 10(p value)), a higher value indicates greater significance and the x-axis is the difference in expression between the two time points as measured in log 2 fold change (log 2FC); orange dots represent genes showing statistically significant changes (FDRp<0.05) and absolute log 2FC≤0.58, blue dots represent genes showing statistically significant changes and absolute log 2FC≥0.58 (FC≥1.5); black dots represent non-significant genes; Panel D shows Volcano plots of pairwise comparisons for DEGs with FC≥2; the y-axis is the negative log 10 of FDR-adjusted p values (−log 10(p value)), a higher value indicates greater significance and the x-axis is the difference in expression between the two time points as measured in log 2 fold change (log 2FC); each gene is represented by one dot in the graph; orange dots represent genes showing statistically significant changes (FDRp<0.05) and absolute log 2FC<1, blue dots represent genes showing statistically significant changes and absolute log 2FC≥1; black dot represent non-significant genes; Panel E shows changes in DEGs number related to DNA repair, chromatin, and cell cycle over time.

FIG. 18 shows DEGs related to apoptosis; tables show gene ontology categories and DEGs related to apoptosis in responders and low-responders.

FIG. 19 shows DEGs related to stem cell proliferation and maintenance in responders.

FIG. 20 shows DEGs associated with G protein-coupled receptor signaling; the tables show function categories and up-regulated genes in responders and low-responders.

FIG. 21, Panel A shows change of the expression in DEGs related to Wnt/β-catenin signaling pathway; bubble graphs representing involvements of the canonical pathway genes determined by IPA (Qiagen, USA) and visualized by R 4.1.0; Panel B shows canonical signaling pathways regulated by DEGs at T3 in low-responders; significant pathways or regulator enrichment were determined activated with positive z-score and inhibited with negative z-score and the FDRp<0.05 (q value), in which z-score is the statistical measure of correlation between relationship direction and gene expression; blue arrow indicates inhibited pathway discussed in the result; Panel C shows validation of selected DEGs by qRT-PCR in two groups of women over time; data was analyzed by pairwise comparison between each time point after treatment versus the baseline (before treatment); error bars representing for the Mean±SEM; *p<0.05, **p<0.01, ***p<0.001; n=10 for responders, n=14 for low-responders.

FIG. 22 shows activation Z-scores for the selected upstream regulators; all activated upstream regulators had Z-score≥2.0, while inhibited regulators had Z-score≤−2.

FIG. 23 shows upstream regulators TGFRB2, TGFBR1, and BRCA1 are predicted activated in the responders; tables show upstream regulators regulated by r-hCG treatment in the responders by IPA analysis.

FIG. 24 shows validation of selected DEGs by qRT-PCR in the two groups over time; pairwise comparison between each time point after treatment versus the baseline (before treatment); error bars representing for Mean±SEM; *p<0.05, **p<0.01, ***p<0.001.

FIG. 25 shows IHC analysis of BRCA1 expression on the breast tissue of BRCA1/2 carriers before and after r-hCG treatment; pictures show one representative example of BRCA1 carriers from each group; magnification, 40× objective; scale bar, 20 μm; the quantification is shown on the right panel; each line represents for one subject; one sample T-test was used for the statistical analysis.

FIG. 26, Panel A shows quantification of primary mammospheres formed by rat mammary epithelial cells; female Sprague-Dawley rats at 50 days old were treated with 100 IU/day r-hCG for 21 days, and let rest for 21 days, then mammary gland 4&5 were dissected, epithelial cells enriched rat mammary cells were isolated and used for mammospheres culture; mammospheres formation was quantified after 7 days of culture; the graph shows the number of mammospheres larger than 50 μm; three rats per group were used for this study; Panel B shows the number of DEGs by microarray analysis. RNA was isolated from primary mammospheres cultured for 7 days; graph represents the number of DEGs relative to control rats; Panel C shows the most enriched GO terms in up or down-regulated genes; bar graph shows the top 12 GO terms in each category; Panel D shows canonical pathways enriched by up or down-regulated genes using IPA; table shows the main pathways and gene name in each pathway; Panel E shows Cd24 expression was reduced in the rat mammary gland ducts 21 days after the last r-hCG treatment (cumulative odd ratio 0.05, 95% CI. 0.009, 0.289) evaluated by IHC staining; five rats were used for the analysis; magnification, 40× objective; scale bar, 20 μm.

FIG. 27, Panel A shows a flow chart of cell treatment; cells were plated and allowed to attach overnight, then treated with 50 IU/ml r-hCG unless indicated, with daily medium change for three days; cells were used at the end of treatment (DO time point), or passaged and used 6 (D6) and 10 (D10) days after the last treatment; Panel B shows fold change relative to the RNA expression in cells treated with vehicle control; paired two-tailed Student's t-test was used for statistical analysis; Panel C shows qRT-PCR for selected genes in cultured cells; BRCA1+/+ or BRCA1mut/+ MCF10A cells were treated with 50 IU/ml r-hCG for 3 days; total RNA was extracted 10 days later (D10) and used for qRT-PCR; three independent passages were used for the analysis; relative fold change to the RNA expression in the cells without r-hCG treatment was plotted; graphs represent Mean±SD; (n=3); paired two-tailed Student's t test was used for statistical analysis; Panel D shows comparison of the RNA expression between BRCA1^{mut/+} and BRCA1+/+ MCF10A cells; Student's t-test was used for the comparison; Panel E shows 30 μg nuclear extract was used for checking BRCA1, BARD1, FOXO3, p53, H3K27me3, SOX7, and SO17, and 40 μg total lysates was used for 0-casein, SFRP4, and TGFβ by WB; Panel F shows cells were treated with r-hCG for 3 days, then irradiated with 2 Gy gamma irradiation (IR), or irradiated with 5 Gy IR 10 days after the last r-hCG treatment; γ-H2AX level was evaluated by WB; graphs in B and D represent Mean±SD; (n=3 passages); immunoblots showed here are representative images from three independent experiments.

FIG. 28, Panel A shows MCF10F cells were treated with r-hCG for 3 days, total lysates was prepared at the end of treatment (DO time pint) and 5 days later (D5); 40 μg total lysates were used for WB; Panel B shows cells were treated with r-hCG for 3 days, 30 μg nuclear extract was used for WB; the increase of BRCA1 and BARD1 was more significant in MCF12A cells (from a nulliparous woman) than in MCF10F and MCF10A (from a parous woman); Panel C shows MCF10F cells were irradiated with 2 Gy gamma irradiation (IR) at the end of 3-day r-hCG treatment (DO), or irradiated 5 days later (D5); γ-H2AX level was evaluated by WB at indicated time points post IR; Panel D shows representative immunofluorescence images and quantification of γ-H2AX foci in MCF10F cells at D5; γ-H2AX was in green color, and nuclei were in blue (DAPI); images were acquired and analyzed using Metamorph software; magnification, 100× objective with oil; *indicates p<0.05.

### DESCRIPTION OF EMBODIMENTS

The terminology used herein is for the purpose of describing particular embodiments only and is not intended to be limiting.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by +10% and remain within the scope of the disclosed embodiments.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The present disclosure provides methods of treating a nulligravid female having a high risk of developing breast cancer. The methods comprise administering human chorionic gonadotropin (hCG) two to four times a week for at least ten weeks, thereby reducing the risk of developing breast cancer. The nulligravid female is without exposure to a contraceptive, in particular a hormonal contraceptive, for at least 21 days prior to administration of the hCG.

In some embodiments, the hCG is administered two to four times a week for at least ten weeks. In some embodiments, the hCG is administered two to four times a week for at least eleven weeks. In some embodiments, the hCG is administered two to four times a week for at least twelve weeks. In some embodiments, the hCG is administered two to four times a week for no more than twelve weeks. In some embodiments, the hCG is administered three times a week for at least eleven weeks. In some embodiments, the hCG is administered three times a week for at least twelve weeks. In some embodiments, the hCG is administered three times a week for no more than twelve weeks.

In some embodiments, the nulligravid female is without exposure to a contraceptive for at least 21 days prior to administration of the hCG. In some embodiments, the nulligravid female is without exposure to a contraceptive for at least 26 days prior to administration of the hCG. In some embodiments, the nulligravid female is without exposure to a contraceptive for at least 30 days prior to administration of the hCG.

In some embodiments, the contraceptive is a hormonal or hormone-based contraceptive. In some embodiments, the contraceptive is an oral hormonal contraceptive, a transdermal contraceptive, or an implanted contraceptive. In some embodiments, the implanted contraceptive is levonorgestrel (LNG) intrauterine device (IUD), LNG-releasing intrauterine system (LNG-IUS), or a progestin IUD.

In some embodiments, the nulligravid female is a carrier of a deleterious mutation in any one or more of BRCA1, BRCA2, PALP2, CHEK2, ATM, TP53, RAD51C, RAD51d, BRIP1, MLH1, MSH2, and MSH6. In some embodiments, the nulligravid female is a carrier of a deleterious mutation in PALP2. In some embodiments, the nulligravid female is a carrier of a deleterious mutation in CHEK2. In some embodiments, the nulligravid female is a carrier of a deleterious mutation in ATM. In some embodiments, the nulligravid female is a carrier of a deleterious mutation in TP53. In some embodiments, the nulligravid female is a carrier of a deleterious mutation in RAD51C. In some embodiments, the nulligravid female is a carrier of a deleterious mutation in RAD51d. In some embodiments, the nulligravid female is a carrier of a deleterious mutation in BRIP1. In some embodiments, the nulligravid female is a carrier of a deleterious mutation in MLH1. In some embodiments, the nulligravid female is a carrier of a deleterious mutation in MSH2. In some embodiments, the nulligravid female is a carrier of a deleterious mutation in MSH6. In some embodiments, the nulligravid female is a carrier of a deleterious mutation in BRCA1 and/or BRCA2. In some embodiments, the nulligravid female is a carrier of a deleterious mutation in BRCA1. In some embodiments, the nulligravid female is a carrier of a deleterious mutation in BRCA2. In some embodiments, the subject possesses any one or more of the other risk factors described herein.

In some embodiments, the nulligravid female has an increased familial risk (e.g., at least one 1^{st} grade relative with breast cancer) of breast cancer with or without having a deleterious mutation in any one or more particular genes. In some embodiments, the nulligravid female has dense breast tissue.

In some embodiments, the nulligravid female is from about 18 years of age to about 40 years of age, from about 18 years of age to about 30 years of age, from about 18 years of age to about 26 years of age, or from about 19 years of age to about 29 years of age. In some embodiments, the nulligravid female is from about 18 years of age to about 40 years of age. In some embodiments, the nulligravid female is from about 18 years of age to about 30 years of age. In some embodiments, the nulligravid female is from about 18 years of age to about 26 years of age. In some embodiments, the nulligravid female is from about 19 years of age to about 29 years of age.

In some embodiments, the hCG is administered in an amount from about 50 μg to about 500 μg, from about 100 μg to about 400 μg, from about 200 μg to about 300 μg, or in an amount of about 250 μg. In some embodiments, the hCG is administered in an amount from about 100 μg to about 400 μg. In some embodiments, the hCG is administered in an amount from about 200 μg to about 300 μg. In some embodiments, the hCG is administered in an amount of about 250 μg. Effective doses of hCG can vary depending upon many different factors, including means of administration, target site, physiological state of the subject, other medications administered, and whether treatment is prophylactic or therapeutic. In some embodiments, the hCG is administered to the nulligravid female in a non-continuous manner, and in particular, only during the luteal phase.

In some embodiments, the hCG is administered subcutaneously, transdermally, intranasally, by an intravaginal ring or implant, or by a controlled release device. In some embodiments, the hCG is administered subcutaneously. In some embodiments, the hCG is administered transdermally. In some embodiments, the hCG is administered intranasally. In some embodiments, the hCG is administered by an intravaginal ring or implant. In some embodiments, the hCG is administered by a controlled release device. In some embodiments, the hCG is administered by subcutaneous injection. In some embodiments, the hCG is administered as a slow release formulation by an implanted controlled release device.

In some embodiments, the hCG is recombinant hCG (rhCG) or urinary hCG, or any therapeutically active peptide thereof. In some embodiments, the hCG is rhCG, or any therapeutically active peptide thereof. In some embodiments, the hCG is rhCG. In some embodiments, the hCG is urinary hCG, or any therapeutically active peptide thereof. In some embodiments, the hCG is urinary hCG. In some embodiments the alpha subunit of hCG comprises the amino acid sequence of Uniprot Protein P01215-1. In some embodiments the beta subunit of hCG comprises the amino acid sequence of any one of Uniprot Protein A6NKQ9-1 and A6NKQ9-2, Uniprot Protein Q6NT52-1, Uniprot Protein P0DN86-1 and P0DN86-2, GenBank Protein AAI06060.1, Uniprot Protein P0DN87-1, or GenBank Protein AAH69526.1,

In some embodiments, the hCG peptide comprises the amino acid sequence Ala Leu Cys Arg Arg Ser Thr Thr Asp Cys Gly Gly Pro Lys Asp His Pro Leu Thr Ser (SEQ ID NO:1), Ser Tyr Ala Val Ala Leu Ser Cys Gln Cys Ala Leu Cys Arg Arg (SEQ ID NO:2), Ser Leu Glu Pro Leu Arg Pro Arg Cys Arg Pro Ile Asn Ala Thr (SEQ ID NO:3), Ser Tyr Ala Val Ala Leu Ser Ala Gln Cys Ala Leu Cys Arg Arg (SEQ ID NO:4), or Ser Phe Pro Val Ala Leu Ser Cys Arg Cys Gly Pro Cys Arg Arg (SEQ ID NO:5). In some embodiments, the hCG peptide comprises the amino acid sequence set forth in SEQ ID NO:1. In some embodiments, the hCG peptide consists of the amino acid sequence set forth in SEQ ID NO: 1. In some embodiments, the hCG peptide comprises the amino acid sequence set forth in SEQ ID NO:2. In some embodiments, the hCG peptide consists of the amino acid sequence set forth in SEQ ID NO:2. In some embodiments, the hCG peptide comprises the amino acid sequence set forth in SEQ ID NO:3. In some embodiments, the hCG peptide consists of the amino acid sequence set forth in SEQ ID NO:3. In some embodiments, the hCG peptide comprises the amino acid sequence set forth in SEQ ID NO: 4. In some embodiments, the hCG peptide consists of the amino acid sequence set forth in SEQ ID NO:4. In some embodiments, the hCG peptide comprises the amino acid sequence set forth in SEQ ID NO:5. In some embodiments, the hCG peptide consists of the amino acid sequence set forth in SEQ ID NO:5. In some embodiments, the hCG peptide comprises an amino acid sequence that is at least about 80%, at least about 85%, at least about 90%, at least about 95%, at least about 96%, at least about 97%, at least about 98%, at least about 99%, or 100% identical to SEQ ID NO:1, SEQ ID NO:2, SEQ ID NO:3, SEQ ID NO:4, or SEQ ID NO:5. In some embodiments, the hCG peptide comprises an amino acid sequence that is at least about 80% identical to these amino acid sequences. In some embodiments, the hCG peptide comprises an amino acid sequence that is at least about 85% identical to these amino acid sequences. In some embodiments, the hCG peptide comprises an amino acid sequence that is at least about 90% identical to these amino acid sequences. In some embodiments, the hCG peptide comprises an amino acid sequence that is at least about 95% identical to these amino acid sequences. In some embodiments, the hCG peptide comprises an amino acid sequence that is at least about 96% identical to these amino acid sequences. In some embodiments, the hCG peptide comprises an amino acid sequence that is at least about 97% identical to these amino acid sequences. In some embodiments, the hCG peptide comprises an amino acid sequence that is at least about 98% identical to these amino acid sequences. In some embodiments, the hCG peptide comprises an amino acid sequence that is at least about 99% identical to these amino acid sequences. In some embodiments, the hCG peptide can be an isolated peptide, a synthesized peptide, or a peptide that forms part of a protein with other peptides.

In some embodiments, the hCG can be formulated in an aqueous buffer. In some embodiments, liquid formulations of a pharmaceutical composition containing hCG prepared in water or other aqueous vehicles can contain various suspending agents such as, for example, methylcellulose, alginates, tragacanth, pectin, kelgin, carrageenan, acacia, polyvinylpyrrolidone, and polyvinyl alcohol, or any combination thereof. Liquid formulations of pharmaceutical compositions can also include solutions, emulsions, syrups and elixirs containing, together with the hCG, wetting agents, sweeteners, and coloring, and flavoring agents. Various liquid and powder formulations of hCG can be prepared by conventional methods.

In some embodiments, liquid formulations of pharmaceutical compositions including hCG for injection can comprise various carriers such as vegetable oils, dimethylacetamide, dimethylformamide, ethyl lactate, ethyl carbonate, isopropyl myristate, ethanol, polyols such as, for example, glycerol, propylene glycol, liquid polyethylene glycol, and the like. In some embodiments, the composition includes a citrate/sucrose/tween carrier. For intravenous injections, water soluble versions of the compositions can be administered by the drip method, whereby a pharmaceutical formulation containing the hCG and a physiologically acceptable excipient can be infused. Physiologically acceptable excipients can include, for example, 5% dextrose, 0.9% saline, Ringer's solution, or other suitable excipients. A suitable insoluble form of the composition can be prepared and administered as a suspension in an aqueous base or a pharmaceutically acceptable oil base, such as an ester of a long chain fatty acid such as, for example, ethyl oleate.

The compositions including hCG can be, for example, injectable solutions, aqueous suspensions or solutions, non-aqueous suspensions or solutions, solid and liquid oral formulations, salves, gels, ointments, intradermal patches, creams, aerosols, lotions, tablets, capsules, sustained release formulations, and the like. In some embodiments, for topical applications, the pharmaceutical compositions can be formulated in a suitable ointment. In some embodiments, a topical semi-solid ointment formulation typically comprises a concentration of the hCG from about 1 to 20%, or from 5 to 10%, in a carrier, such as a pharmaceutical cream base. Some examples of formulations of a composition for topical use include, but are not limited to, drops, tinctures, lotions, creams, solutions, and ointments containing the active ingredient and various supports and vehicles.

The methods described above for administration of hCG can be adapted to administration of a therapeutically active peptide of hCG as needed.

The present disclosure also provides methods of monitoring the efficacy of treatment of a subject having breast cancer or having a high risk of developing breast cancer. The methods comprise a) obtaining or having obtained a biological sample from the subject prior to treatment initiation (T1) to provide a baseline expression of a panel of genes from the biological sample. The methods also comprise b) obtaining or having obtained a biological sample from the subject after treatment completion (T2). The methods also comprise c) obtaining or having obtained a biological sample from the subject about 6 months or later after treatment completion (T3). The methods also comprise performing a gene expression assay on the T1, T2, and T3 samples to identify a set of differentially expressed genes from the biological sample. Increased expression in at least 10 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 5 of the following genes: FBL, FZD1, FZD7, HMAG1, ITGB4, KIT, LIG1, miR182, NPM2, MMP7, MYC, MYCL, PADI2, PROM1, RPS6, RPS12, RPS18, RPS19, SOX9, and SOX10, in the biological sample at T2 compared to T1 indicates that the treatment is efficacious, and a lack of the differentially expressed gene profile indicates that the treatment is non-efficacious. Alternately, increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, KIT, ID4, miR182, MMP7, MYC, PADI2, SOX9, and SOX10, in the biological sample at T3 compared to T1 indicates that the treatment is efficacious, and a lack of the differentially expressed gene profile indicates that the treatment is non-efficacious. Suitable gene expression assays, which may be used to determine an increase or decrease in the level of expression of a particular gene, are described in, for example, the Examples section below.

In some embodiments, the subjects having breast cancer are BRCA1/2 mutation carriers. In some embodiments, the subjects having breast cancer are BRCA1/2 mutation carriers that have not yet developed breast cancer.

In some embodiments, the biological sample is breast tissue, blood, or urine, or any combination thereof. In some embodiments, the biological sample is breast tissue. In some embodiments, the biological sample is blood. In some embodiments, the biological sample is urine. Biological samples can be obtained using a variety of methods including drawing blood or collecting a urine sample from a subject. Tissue samples can be obtained using standard techniques including excisions, punctures, and aspiration, or other methods. In some embodiments, a sample of breast tissue is obtained by making an incision and taking one or more core samples. In some embodiments a SPIROTOME® biopsy may be performed on a subject as described in the Examples section below.

In some embodiments, the biological sample for identification of the baseline expression of the panel of genes is obtained from the subject about 3 months prior to treatment initiation. In some embodiments, the biological sample for identification of the baseline expression of the panel of genes is obtained from the subject during a period of time when the subject is taking no contraceptive, such as between T1 and about 21 days prior to T1.

In some embodiments, the biological sample obtained from the subject after treatment completion in step b) is obtained from the subject from about 1 day to about 7 days after treatment completion. In some embodiments, the biological sample obtained from the subject after treatment completion in step b) is obtained from the subject within 3 days after treatment completion. In some embodiments, the biological sample obtained from the subject after treatment completion in step b) is obtained from the subject within one or two days after treatment completion.

In some embodiments, the treatment comprises administering hCG to the subject. In some embodiments, the hCG is rhCG or urinary hCG, or any therapeutically active peptide thereof. In some embodiments, the hCG is any of the hCG molecules or therapeutically active peptides thereof described herein administered in any of the dosing regimens described herein. In some embodiments, the hCG treatment can include additional other compounds.

In some embodiments, increased expression in at least 20 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 8 of the following genes: FBL, FZD1, FZD7, HMAG1, ITGB4, KIT, LIG1, miR182, NPM2, MMP7, MYC, MYCL, PADI2, PROM1, RPS6, RPS12, RPS18, RPS19, SOX9, and SOX10, in the biological sample at T2 compared to T1 indicates that the treatment is efficacious, and a lack of the differentially expressed gene profile indicates that the treatment is non-efficacious. Alternately, increased expression in at least 20 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 5 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and a lack of the differentially expressed gene profile indicates that the treatment is non-efficacious.

In some embodiments, increased expression in at least 30 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 10 of the following genes: FBL, FZD1, FZD7, HMAG1, ITGB4, KIT, LIG1, miR182, NPM2, MMP7, MYC, MYCL, PADI2, PROM1, RPS6, RPS12, RPS18, RPS19, SOX9, and SOX10, in the biological sample at T2 compared to T1 indicates that the treatment is efficacious, and a lack of the differentially expressed gene profile indicates that the treatment is non-efficacious. Alternately, increased expression in at least 30 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 6 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and a lack of the differentially expressed gene profile indicates that the treatment is non-efficacious.

In some embodiments, increased expression in at least 40 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 12 of the following genes: FBL, FZD1, FZD7, HMAG1, ITGB4, KIT, LIG1, miR182, NPM2, MMP7, MYC, MYCL, PADI2, PROM1, RPS6, RPS12, RPS18, RPS19, SOX9, and SOX10, in the biological sample at T2 compared to T1 indicates that the treatment is efficacious, and a lack of the differentially expressed gene profile indicates that the treatment is non-efficacious. Alternately, increased expression in at least 40 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 7 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and a lack of the differentially expressed gene profile indicates that the treatment is non-efficacious.

In some embodiments, increased expression in at least 50 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 15 of the following genes: FBL, FZD1, FZD7, HMAG1, ITGB4, LIG1, KIT, miR182, NPM2, MMP7, MYC, MYCL, PADI2, PROM1, RPS6, RPS12, RPS18, RPS19, SOX9, and SOX10, in the biological sample at T2 compared to T1 indicates that the treatment is efficacious, and a lack of the differentially expressed gene profile indicates that the treatment is non-efficacious. Alternately, increased expression in at least 50 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 8 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and a lack of the differentially expressed gene profile indicates that the treatment is non-efficacious.

In some embodiments, increased expression of the following nine genes: BRCA1, FOXO3, HMOX1, SFRP4, SOX7, SOX17, SOX18, TGFB1, and TGFB3, and/or decreased expression of the following six genes: HMAG1, KIT, miR182, MMP7, MYC, SOX9, and ID4, in the biological sample at T2 compared to T1 indicates that the treatment is efficacious, and a lack of the differentially expressed gene profile indicates that the treatment is non-efficacious. Alternately, increased expression of the following nine genes: BRCA1, FOXO3, HMOX1, SFRP4, SOX7, SOX17, SOX18, TGFB1, and TGFB3, and/or decreased expression of the following six genes: HMAG1, KIT, miR182, MMP7, MYC, SOX9, and ID4, in the biological sample at T3 compared to T1 indicates that the treatment is efficacious, and a lack of the differentially expressed gene profile indicates that the treatment is non-efficacious.

In some embodiments, upon an indication of efficacious treatment, the treatment can be discontinued. In some embodiments, upon an indication of non-efficacious treatment, the treatment can be altered to a different treatment. For example, for subjects that do not sufficiently respond to treatment with hCG by producing the recited gene expression profiles described herein, 1) the administration of hCG can continue without interruption until a sufficient response is generated, 2) hCG treatment can be suspended for a particular period of time followed by a second round of hCG administration, 3) the dosage of hCG can be increased, or 4) a different anti-cancer therapeutic regimen can be sought. To determine how much to increase the dosage of hCG after 12 weeks of administration, for a normal pregnancy, the hCG blood levels are high throughout the 40 weeks of pregnancy, with a peak (up to 210,000 U/L) occurring around 12 weeks after the last menstrual period. The increase in the dosage of hCG can be in amount to mimic the hCG blood levels observed during pregnancy.

In some embodiments, the subject is a carrier of a deleterious mutation in any one or more of BRCA1, BRCA2, PALP2, CHEK2, ATM, TP53, RAD51C, RAD51d, BRIP1, MLH1, MSH2, and MSH6. In some embodiments, the subject is a carrier of a deleterious mutation in PALP2. In some embodiments, the subject is a carrier of a deleterious mutation in CHEK2. In some embodiments, the subject is a carrier of a deleterious mutation in ATM. In some embodiments, the subject is a carrier of a deleterious mutation in TP53. In some embodiments, the subject is a carrier of a deleterious mutation in RAD51C. In some embodiments, the subject is a carrier of a deleterious mutation in RAD51d. In some embodiments, the subject is a carrier of a deleterious mutation in BRIP1. In some embodiments, the subject is a carrier of a deleterious mutation in MLH1. In some embodiments, the subject is a carrier of a deleterious mutation in MSH2. In some embodiments, the subject is a carrier of a deleterious mutation in MSH6. In some embodiments, the subject is a carrier of a deleterious mutation in BRCA1 and/or BRCA2. In some embodiments, the subject is a carrier of a deleterious mutation in BRCA1. In some embodiments, the subject is a carrier of a deleterious mutation in BRCA2. In some embodiments, the subject possesses any one or more of the other risk factors described herein.

In some embodiments, the subject having breast cancer or having a high risk of developing breast cancer is a nulligravid female without exposure to a contraceptive for at least 21 days prior to administration of the hCG. In some embodiments, the subject having breast cancer or having a high risk of developing breast cancer is a nulligravid female is without exposure to a contraceptive for at least 26 days prior to administration of the hCG. In some embodiments, the subject having breast cancer or having a high risk of developing breast cancer is a nulligravid female is without exposure to a contraceptive for at least 30 days prior to administration of the hCG.

In some embodiments, the contraceptive is a hormonal contraceptive. In some embodiments, the contraceptive is an oral hormonal contraceptive, a transdermal contraceptive, or an implanted contraceptive. In some embodiments, the implanted contraceptive is levonorgestrel (LNG) intrauterine device (IUD), LNG-releasing intrauterine system (LNG-IUS), or a progestin IUD.

In some embodiments, the subject is a female is from about 18 years of age to about 40 years of age, from about 18 years of age to about 30 years of age, from about 18 years of age to about 26 years of age, or from about 19 years of age to about 29 years of age. In some embodiments, the subject is a female is from about 18 years of age to about 40 years of age. In some embodiments, the subject is a female is from about 18 years of age to about 30 years of age. In some embodiments, the subject is a female is from about 18 years of age to about 26 years of age. In some embodiments, the subject is a female is from about 19 years of age to about 29 years of age.

In any of the embodiments described herein, an “increased expression” of any of the genes set forth herein means at least a 2% increase, at least a 5% increase, at least a 10% increase, at least a 15% increase, or at least a 20% increase in the level of DNA or RNA for the gene. Likewise, in any of the embodiments described herein, a “decreased expression” of any of the genes set forth herein means at least a 2% decrease, at least a 5% decrease, at least a 10% decrease, at least a 15% decrease, or at least a 20% decrease in the level of DNA or RNA for the gene. The increased expression or decreased expression can be determined by any art accepted methodology, such as, for example, TaqMan Gene Expression Assay (Thermo Fisher Scientific).

The present disclosure also provides methods of monitoring the efficacy of treatment of a subject having breast cancer or having a high risk of developing breast cancer. The methods comprise a) obtaining or having obtained a biological sample from the subject prior to treatment initiation (T1) to provide a baseline expression of a panel of genes from the biological sample. The methods also comprise b) obtaining or having obtained a biological sample from the subject after treatment initiation (T1). The methods also comprise c) performing a gene expression assay on the two samples to identify a set of differentially expressed genes from the biological sample. Increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and a lack of the differentially expressed gene profile indicates that the treatment is not yet efficacious.

In some embodiments, the biological sample is breast tissue, blood, or urine, or any combination thereof. In some embodiments, the biological sample is breast tissue. In some embodiments, the biological sample is blood. In some embodiments, the biological sample is urine. Biological samples can be obtained using a variety of methods including drawing blood or collecting a urine sample from a subject. Tissue samples can be obtained using standard techniques including excisions, punctures, and aspiration, or other methods. In some embodiments, a sample of breast tissue is obtained by making an incision and taking one or more core samples. In some embodiments a SPIROTOME® biopsy may be performed on a subject as described in the Examples section below.

In some embodiments, the biological sample for identification of the baseline expression of the panel of genes is obtained from the subject about 3 months prior to treatment initiation.

In some embodiments, the biological sample obtained from the subject after treatment initiation in step b) is obtained from the subject from about 1 month to about 9 months after treatment initiation. In some embodiments, the biological sample obtained from the subject after treatment initiation in step b) is obtained from the subject from about 3 months to about 9 months after treatment initiation. In some embodiments, the biological sample obtained from the subject after treatment initiation in step b) is obtained from the subject from about 6 months to about 9 months after treatment initiation.

In some embodiments, the treatment comprises administering hCG to the subject. In some embodiments, the hCG is rhCG or urinary hCG, or any therapeutically active peptide thereof. In some embodiments, the hCG is any of the hCG molecules or therapeutically active peptides thereof described herein administered in any of the dosing regimens described herein.

In some embodiments, increased expression in at least 20 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 5 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and a lack of the differentially expressed gene profile indicates that the treatment is not yet efficacious.

In some embodiments, increased expression in at least 30 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 6 of the following genes: EYA2, FZD1, HMGA1, TD4, KIT, miR182, MMP7, MYC, PADI2, SOX9, and SOX10, in the biological sample obtained in step b) compared to step a) indicates that the treatment is efficacious, and a lack of the differentially expressed gene profile indicates that the treatment is not yet efficacious.

In some embodiments, increased expression in at least 40 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 7 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and a lack of the differentially expressed gene profile indicates that the treatment is not yet efficacious.

In some embodiments, increased expression in at least 50 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 8 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and a lack of the differentially expressed gene profile indicates that the treatment is not yet efficacious.

In some embodiments, increased expression of the following nine genes: BRCA1, FOXO3, HMOX1, SFRP4, SOX7, SOX17, SOX18, TGFB1, and TGFB3, and/or decreased expression of the following six genes: HMAG1, KIT, miR182, MMP7, MYC, SOX9, and ID4, in the biological sample obtained in step b) compared to step a) indicates that the treatment is efficacious, and a lack of the differentially expressed gene profile indicates that the treatment is not yet efficacious.

In some embodiments, upon an indication of efficacious treatment, the treatment can be discontinued. In some embodiments, upon an indication of non-efficacious treatment, the treatment can be altered to a different treatment. For example, for subjects that do not sufficiently respond to treatment with hCG by producing the recited gene expression profiles described herein, 1) the administration of hCG can continue without interruption until a sufficient response is generated, 2) hCG treatment can be suspended for a particular period of time followed by a second round of hCG administration, 3) the dosage of hCG can be increased, or 4) a different anti-cancer therapeutic regimen can be sought.

In some embodiments, the subject is a carrier of a deleterious mutation in any one or more of BRCA1, BRCA2, PALP2, CHEK2, ATM, TP53, RAD51C, RAD51d, BRIP1, MLH1, MSH2, and MSH6. In some embodiments, the subject is a carrier of a deleterious mutation in PALP2. In some embodiments, the subject is a carrier of a deleterious mutation in CHEK2. In some embodiments, the subject is a carrier of a deleterious mutation in ATM. In some embodiments, the subject is a carrier of a deleterious mutation in TP53. In some embodiments, the subject is a carrier of a deleterious mutation in RAD51C. In some embodiments, the subject is a carrier of a deleterious mutation in RAD51d. In some embodiments, the subject is a carrier of a deleterious mutation in BRIP1. In some embodiments, the subject is a carrier of a deleterious mutation in MLH1. In some embodiments, the subject is a carrier of a deleterious mutation in MSH2. In some embodiments, the subject is a carrier of a deleterious mutation in MSH6. In some embodiments, the subject is a carrier of a deleterious mutation in BRCA1 and/or BRCA2. In some embodiments, the subject is a carrier of a deleterious mutation in BRCA1. In some embodiments, the subject is a carrier of a deleterious mutation in BRCA2. In some embodiments, the subject possesses any one or more of the other risk factors described herein.

In some embodiments, the subject having breast cancer or having a high risk of developing breast cancer is a nulligravid female without exposure to a contraceptive for at least 21 days prior to administration of the hCG. In some embodiments, the subject having breast cancer or having a high risk of developing breast cancer is a nulligravid female is without exposure to a contraceptive for at least 26 days prior to administration of the hCG. In some embodiments, the subject having breast cancer or having a high risk of developing breast cancer is a nulligravid female is without exposure to a contraceptive for at least 30 days prior to administration of the hCG.

In some embodiments, the contraceptive is an oral hormonal contraceptive, a transdermal contraceptive, or an implanted contraceptive. In some embodiments, the implanted contraceptive is levonorgestrel (LNG) intrauterine device (IUD), LNG-releasing intrauterine system (LNG-IUS), or a progestin IUD.

In some embodiments, the subject is a female is from about 18 years of age to about 40 years of age, from about 18 years of age to about 30 years of age, from about 18 years of age to about 26 years of age, or from about 19 years of age to about 29 years of age. In some embodiments, the subject is a female is from about 18 years of age to about 40 years of age. In some embodiments, the subject is a female is from about 18 years of age to about 30 years of age. In some embodiments, the subject is a female is from about 18 years of age to about 26 years of age. In some embodiments, the subject is a female is from about 19 years of age to about 29 years of age.

The present disclosure provides methods of determining whether a subject is at risk of developing breast cancer. The methods comprise obtaining or having obtained a biological sample from the subject and performing a gene expression assay to identify an expression profile of a panel of genes from the biological sample. Increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer. When the subject does not have the expression profile, the subject is at higher risk of developing breast cancer.

In some embodiments, increased expression in at least 20 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 5 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer. When the subject does not have the expression profile, the subject is at higher risk of developing breast cancer.

In some embodiments, increased expression in at least 30 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 6 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer. When the subject does not have the expression profile, the subject is at higher risk of developing breast cancer.

In some embodiments, increased expression in at least 40 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 7 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer. When the subject does not have the expression profile, the subject is at higher risk of developing breast cancer.

In some embodiments, increased expression in at least 50 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 8 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer. When the subject does not have the expression profile, the subject is at higher risk of developing breast cancer.

In some embodiments, increased expression of the following nine genes: BRCA1, FOXO3, HMOX1, SFRP4, SOX7, SOX17, SOX18, TGFB1, and TGFB3, and/or decreased expression of the following six genes: HMAG1, KIT, miR182, MMP7, MYC, SOX9, and ID4, in the biological sample obtained from the subject compared to a control breast cancer expression profile indicates that the subject has a lower risk of developing breast cancer. When the subject does not have the expression profile, the subject is at higher risk of developing breast cancer.

In some embodiments, the control breast cancer expression profile is derived from a subject having breast cancer.

In some embodiments, the biological sample is breast tissue, blood, or urine, or any combination thereof. In some embodiments, the biological sample is breast tissue. In some embodiments, the biological sample is blood. In some embodiments, the biological sample is urine. Biological samples can be obtained using a variety of methods including drawing blood or collecting a urine sample from a subject. Tissue samples can be obtained using standard techniques including excisions, punctures, and aspiration, or other methods. In some embodiments, a sample of breast tissue is obtained by making an incision and taking one or more core samples. In some embodiments a SPIROTOME® biopsy may be performed on a subject as described in the Examples section below.

In some embodiments, the subject is a female is from about 18 years of age to about 40 years of age, from about 18 years of age to about 30 years of age, from about 18 years of age to about 26 years of age, or from about 19 years of age to about 29 years of age. In some embodiments, the subject is a female is from about 18 years of age to about 40 years of age. In some embodiments, the subject is a female is from about 18 years of age to about 30 years of age. In some embodiments, the subject is a female is from about 18 years of age to about 26 years of age. In some embodiments, the subject is a female is from about 19 years of age to about 29 years of age.

In some embodiments, when the subject does not have the recited gene expression profile, the subject is further treated to prevent the development of breast cancer. In some embodiments, the treatment can be any of the treatments with any of the hCG molecules described herein by any of the dosing regimens described herein. In some embodiments, the treatment comprises administering from about 50 μg to about 500 μg of hCG two to four times a week for at least ten weeks. In some embodiments, the hCG is administered two to four times a week for at least eleven weeks. In some embodiments, the hCG is administered two to four times a week for at least twelve weeks. In some embodiments, the hCG is administered two to four times a week for no more than twelve weeks. In some embodiments, the hCG is administered three times a week for at least eleven weeks. In some embodiments, the hCG is administered three times a week for at least twelve weeks. In some embodiments, the hCG is administered three times a week for no more than twelve weeks. In some embodiments, the hCG is administered in an amount from about 100 μg to about 400 μg. In some embodiments, the hCG is administered in an amount from about 200 μg to about 300 μg. In some embodiments, the hCG is administered in an amount of about 250 μg.

In some embodiments, the subject is a carrier of a deleterious mutation in any one or more of BRCA1, BRCA2, PALP2, CHEK2, ATM, TP53, RAD51C, RAD51d, BRIP1, MLH1, MSH2, and MSH6. In some embodiments, the subject is a carrier of a deleterious mutation in PALP2. In some embodiments, the subject is a carrier of a deleterious mutation in CHEK2. In some embodiments, the subject is a carrier of a deleterious mutation in ATM. In some embodiments, the subject is a carrier of a deleterious mutation in TP53. In some embodiments, the subject is a carrier of a deleterious mutation in RAD51C. In some embodiments, the subject is a carrier of a deleterious mutation in RAD51d. In some embodiments, the subject is a carrier of a deleterious mutation in BRIP1. In some embodiments, the subject is a carrier of a deleterious mutation in MLH1. In some embodiments, the subject is a carrier of a deleterious mutation in MSH2. In some embodiments, the subject is a carrier of a deleterious mutation in MSH6. In some embodiments, the subject is a carrier of a deleterious mutation in BRCA1 and/or BRCA2. In some embodiments, the subject is a carrier of a deleterious mutation in BRCA1. In some embodiments, the subject is a carrier of a deleterious mutation in BRCA2. In some embodiments, the subject possesses any one or more of the other risk factors described herein.

In some embodiments, the subject is a nulligravid female without exposure to a contraceptive for at least 21 days prior to administration of the hCG. In some embodiments, the subject is a nulligravid female is without exposure to a contraceptive for at least 26 days prior to administration of the hCG. In some embodiments, the subject is a nulligravid female is without exposure to a contraceptive for at least 30 days prior to administration of the hCG.

In some embodiments, the contraceptive is an oral hormonal contraceptive, a transdermal contraceptive, or an implanted contraceptive. In some embodiments, the implanted contraceptive is levonorgestrel (LNG) intrauterine device (IUD), LNG-releasing intrauterine system (LNG-IUS), or a progestin IUD.

The present disclosure also provides hCG, or any therapeutically active peptide thereof, for use in treating a nulligravid female having a high risk of developing breast cancer. The treating comprises administering hCG, or any therapeutically active peptide thereof, two to four times a week for at least ten weeks, thereby reducing the risk of developing breast cancer. The nulligravid female is without exposure to a contraceptive, in particular, a hormonal contraceptive, for at least 21 days prior to administration of the hCG.

The present disclosure also provides use of hCG, or any therapeutically active peptide thereof, in the preparation of a medicament for use in treating a nulligravid female having a high risk of developing breast cancer. The use comprises administering hCG, or any therapeutically active peptide thereof, two to four times a week for at least ten weeks, thereby reducing the risk of developing breast cancer. The nulligravid female is without exposure to a contraceptive for at least 21 days prior to administration of the hCG.

In some embodiments, the hCG is administered two to four times a week for at least ten weeks. In some embodiments, the hCG is administered two to four times a week for at least eleven weeks. In some embodiments, the hCG is administered two to four times a week for at least twelve weeks. In some embodiments, the hCG is administered two to four times a week for no more than twelve weeks. In some embodiments, the hCG is administered three times a week for at least eleven weeks. In some embodiments, the hCG is administered three times a week for at least twelve weeks. In some embodiments, the hCG is administered three times a week for no more than twelve weeks. Administration can be in a continuous mode or can be non-continuous, so as, for example, where hCG is administered only during the luteal phase.

In some embodiments, the nulligravid female is without exposure to a contraceptive for at least 21 days prior to administration of the hCG. In some embodiments, the nulligravid female is without exposure to a contraceptive for at least 26 days prior to administration of the hCG. In some embodiments, the nulligravid female is without exposure to a contraceptive for at least 30 days prior to administration of the hCG.

In some embodiments, the contraceptive is a hormone-based or hormonal contraceptive. In some embodiments, the contraceptive is an oral hormonal contraceptive, a transdermal contraceptive, or an implanted contraceptive. In some embodiments, the implanted contraceptive is LNG IUD, LNG-IUS, or a progestin IUD.

In some embodiments, the nulligravid female is a carrier of a deleterious mutation in any one or more of BRCA1, BRCA2, PALP2, CHEK2, ATM, TP53, RAD51C, RAD51d, BRIP1, MLH1, MSH2, and MSH6. In some embodiments, the nulligravid female is a carrier of a deleterious mutation in PALP2. In some embodiments, the nulligravid female is a carrier of a deleterious mutation in CHEK2. In some embodiments, the nulligravid female is a carrier of a deleterious mutation in ATM. In some embodiments, the nulligravid female is a carrier of a deleterious mutation in TP53. In some embodiments, the nulligravid female is a carrier of a deleterious mutation in RAD51C. In some embodiments, the nulligravid female is a carrier of a deleterious mutation in RAD51d. In some embodiments, the nulligravid female is a carrier of a deleterious mutation in BRIP1. In some embodiments, the nulligravid female is a carrier of a deleterious mutation in MLH1. In some embodiments, the nulligravid female is a carrier of a deleterious mutation in MSH2. In some embodiments, the nulligravid female is a carrier of a deleterious mutation in MSH6. In some embodiments, the nulligravid female is a carrier of a deleterious mutation in BRCA1 and/or BRCA2. In some embodiments, the nulligravid female is a carrier of a deleterious mutation in BRCA1. In some embodiments, the nulligravid female is a carrier of a deleterious mutation in BRCA2. In some embodiments, the subject possesses any one or more of the other risk factors described herein.

In some embodiments, the nulligravid female is from about 18 years of age to about 40 years of age, from about 18 years of age to about 30 years of age, from about 18 years of age to about 26 years of age, or from about 19 years of age to about 29 years of age. In some embodiments, the nulligravid female is from about 18 years of age to about 40 years of age. In some embodiments, the nulligravid female is from about 18 years of age to about 30 years of age. In some embodiments, the nulligravid female is from about 18 years of age to about 26 years of age. In some embodiments, the nulligravid female is from about 19 years of age to about 29 years of age.

In some embodiments, the hCG is administered in an amount from about 50 μg to about 500 μg, from about 100 μg to about 400 μg, from about 200 μg to about 300 μg, or in an amount of about 250 μg. In some embodiments, the hCG is administered in an amount from about 100 μg to about 400 μg. In some embodiments, the hCG is administered in an amount from about 200 μg to about 300 μg. In some embodiments, the hCG is administered in an amount of about 250 μg. In some embodiments, the hCG is administered to the nulligravid female during the luteal phase.

In some embodiments, the hCG is administered subcutaneously, transdermally, intranasally, by an intravaginal ring or implant, or by a controlled release device. In some embodiments, the hCG is administered subcutaneously. In some embodiments, the hCG is administered transdermally. In some embodiments, the hCG is administered intranasally. In some embodiments, the hCG is administered by an intravaginal ring or implant. In some embodiments, the hCG is administered by a controlled release device. In some embodiments, the hCG is administered by subcutaneous injection. In some embodiments, the hCG is administered as a slow release formulation by an implanted controlled release device.

In some embodiments, the treatment comprises administering hCG to the subject. In some embodiments, the hCG is rhCG or urinary hCG, or any therapeutically active peptide thereof. In some embodiments, the hCG is any of the hCG molecules or therapeutically active peptides thereof described herein administered in any of the dosing regimens described herein.

The present disclosure also provides an in vitro method of monitoring the efficacy of treatment of a subject having breast cancer or having a high risk of developing breast cancer, the method comprising: a) prior to treatment initiation (T1), performing a gene expression assay to identify a baseline expression of a panel of genes from a biological sample from the subject; b) after treatment completion (T2), performing a gene expression assay to identify a set of differentially expressed genes from a biological sample from the subject; and c) about 6 months or later after treatment completion (T3), performing a gene expression assay to identify a set of differentially expressed genes from a biological sample from the subject; wherein increased expression in at least 10, 15, 20, 25, 30, 35, 40, 45, 50, or all of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 5, 10, 15, or all of the following genes: FBL, FZD1, FZD7, HMAG1, ITGB4, KIT, LIG1, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 10, 15, 20, 25, 30, 35, 40, 45, 50, or all of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4, 5, 6, 7, 8, 9, of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and wherein a lack of the differentially expressed gene profile indicates that the treatment is non-efficacious.

In some embodiments, timepoint T1 is about 3 months prior to treatment initiation, in particular at least 21 days prior to treatment initiation. In some embodiments, timepoint T2 is from about 1 day to about 7 days after treatment completion, in particular within 3 days after treatment completion, more in particular within one or two days after treatment completion.

The present disclosure also provides an in vitro method of monitoring the efficacy of treatment of a subject having breast cancer or having a high risk of developing breast cancer, the method comprising: a) prior to treatment initiation (T1), performing a gene expression assay to identify a baseline expression of a panel of genes from the biological sample from the subject; and b) after treatment initiation (T1), performing a gene expression assay to identify a set of differentially expressed genes from the biological sample from the subject; wherein increased expression in at least 10, 15, 20, 25, 30, 35, 40, 45, 50, or all of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4, 5, 6, 7, 8, 9, or 10 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

In some embodiments, the timepoint T1 in step a) is about 3 months prior to treatment initiation, and the timepoint in step b) is from about 1 month to about 9 months after treatment initiation, in particular from about 3 months to about 9 months after treatment initiation, more in particular from about 6 months to about 9 months after treatment initiation.

In some embodiments of the above methods and upon an indication of efficacious treatment, the treatment can be discontinued or in the alternative the treatment can be altered to a different treatment. In particular, the treatment is with hCG as disclosed herein before.

The present disclosure also provides an in vitro method of determining whether a subject is at risk of developing breast cancer, the method comprising performing a gene expression assay to identify an expression profile of a panel of genes from a biological sample of the subject; wherein increased expression in at least 10, 15, 20, 25, 30, 35, 40, 45, 50, or all of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4, 5, 6, 7, 8, 9, or 10 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer; and when the subject does not have the expression profile, the subject is at higher risk of developing breast cancer.

In particular, the biological sample is breast tissue, blood, or urine, or any combination thereof. More in particular, the sample is breast tissue.

The present disclosure also provides a method or assay for determining the expression of at least two genes in a biological sample from a subject; wherein at least one gene is selected from the group consisting of ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2; and wherein at least one gene is selected from the group consisting of FBL, FZD1, FZD7, HMAG1, ITGB4, LIG1, miR182, NPM2, MMP7, MYC, MYCL, PADI2, PROM1, RPS6, RPS12, RPS18, RPS19, SOX9, and SOX10. In a further embodiment, the method or assay determines at least 5 genes, at least 10 genes, at least 15 genes, at least 20 genes, at least 30 genes, at least 40 genes, at least 50 genes or all of said genes.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: General Methodology

### A. Clinical Trial Using R-HCG

### Study Design and Patient Samples Collection

In brief, thirty-three women with germline BRCA1/2 mutation but free of breast cancer, at the age of 18 to 29 years old, were included in this study using criteria described in ClinicalTrial.gov (NCT0349569). Participants received a subcutaneous injection of 250 μg r-hCG (OVIDREL, 250 μg/0.5 ml; EMD Serono Inc., Rockland, MA, USA) three times a week (Monday, Wednesday and Friday) for 3 months. Breast tissue biopsies were obtained using Spirotome® biopsy before (time point T1) and after 3 months of r-hCG injection (T2), as well as 6 months after the last r-hCG administration (T3). The fragment of breast tissue for histology analysis was fixed in 70% ethanol, and the fragment for RNA analysis was stored in RNAlater RNA Stabilization Reagent.

### RNA-Sequencing (RNA-Seq) and Analysis

RNA samples from 25 women were used for RNA-seq. Library construction and sequencing were carried out by the BGI Company in Hong Kong.

RNA isolation and RNA-sequencing (RNA-seq): For RNA-seq, total RNA was extracted using RNeasy Lipid Tissue Mini kit (Qiagen, US) within a month after all samples were received. Library construction was performed using PE100 strand-specific library preparation for eukaryote (BGI, CA, USA) to generate DNA nanoball (DNB), which had more than 300 copies of one molecule. The DNBs were loaded into the patterned nanoarray and pair end 100 bases reads were generated by combinatorial Probe-Anchor Synthesis (cPAS) on the BGISEQ-500 platform (BGI, CA, USA) with more than 60 million reads delivered to each of the samples.

RNA-seq analysis: Whole transcriptome profiles of breast tissues were generated for 25 women using RNA-seq. Three transcriptome profiles that represent three time points were generated for each woman. In total, there were 150 files for this analysis with each containing 128-199 million reads. All the raw reads were quality controlled by FastQC (Babraham Bioinformatics, UK), and filtered using CLC Genomics Workbench version 12.0.3 (Qiagen, US) prior to being subjected to alignment. FastQC results were aggregated by MultiQC (world wide web at “pypi.python.org/pypi/multiqc”). The human reference genome GRCh38 was used for read aligning. The mapping rate ranged from 98% to 99% for all the samples. CLC Genomics Workbench version 12.0.3 (Qiagen, US) was used for the analysis. Differential expression analyses were performed using a generalized linear model (GLM) linked to the negative binomial distribution (Robinson et al., Biostatistics, 2008, 9, 321-32).

Pairwise comparisons for each woman for the data was conducted at three time points: T1, T2, and T3. Genes with absolute fold change (FC) larger than 1.5 (FC>1.5) and a false discovery rate (FDR)-adjusted p-value less than 0.05 (FDRp<0.05) were considered as differentially expressed genes (DEGs). The 25 women were divided into two groups according to the contraceptives use: 11 women who never used contraceptives, or stopped oral contraceptives more than 30 days prior to r-hCG treatment were named responders, and 14 women who stopped oral contraceptives less than 30 days prior to r-hCG treatment, or used contraceptives during the study, were named low-responders.

### Gene Enrichment Analysis

Gene Ontology (GO) Enrichment Analysis for the DEGs were analyzed via the Reactome Knowledgebase (world wide web at “reactome.org”) (Fabregat et al., Nucleic Acids Res., 2016, 44, D481-487), ShinyGO v0.61 (Ge et al., Bioinformatics, 2020, 36, 2628-2629), DAVID toolkit (Huang et al., Genome Biol., 2007, 8, R183), and Benjamini-Hochberg correction with cutoff p<0.05. Comparison between the DEGs herein and database of GO consortium (world wide web at “geneontology.org/”) (Ashburner et al., Nat. Genet., 2000, 25, 25-29) were performed to obtain all known genes associated with DNA damage repairs, chromatin remodeling, G protein-coupled receptor (GPCR) and cell cycle. Signaling pathways and biological processes with FDRp<0.05 were considered significant. Canonical pathways and upstream regulators were analyzed using Ingenuity Pathway Analysis (IPA, Qiagen, USA) with adjusted p value <0.05 and Z-score>2.0 for activated pathway/regulator and Z-score <−2.0 for inhibited pathway/regulator (Krämer et al., Bioinformatics, 2014, 30, 523-30). Interactive networks of target genes and related regulator/pathway were built using IPA. Venn diagram, volcano plots and heatmaps were generated using β version 4.0.3 (world wide web at “r-project.org/”) with package VennDiagram, ggplot2, and pheatmap. Chord diagrams for relationships between target genes and related signaling pathways at different time points for each group of women were generated using Circos (Krzywinski et al., Genome Res., 2009, 19, 1639-1645.

Analysis of GPCR Signaling Related Genes For 75 GPCR signaling related genes, the mean expression for responders and low-responders was presented in log 2 value. The change of gene expression was calculated by the formula: log 2 (fold change)=mean expression at T2 or T3−mean expression at T1.
Quantitative RT-PCR (qRT-PCR) Validation

TaqMan gene expression assays were used for the analysis of genes of interest. Briefly, total RNA of breast tissues was extracted using AllPrep DNA/RNA Mini Kit (#80204, Qiagen). Extraction of total RNA including miRNA was performed using miRNeasy Mini Kit (#217004, Qiagen). TaqMan gene expression assays (Thermo Fisher Scientific) were used for the analysis of genes of interest. 12 to 16 ng RNA was used for each reaction in 384 PCR plate with three replications for each sample. QuantStudio 6 Pro Real-Time PCR system was used to run the PCR.

### Probes Used for Quantitative RT-PCR

| Genes | Assay ID | Supplier |
|---|---|---|
| BRCA1 | Hs01556193_m1 | Thermo Fisher Scientific |
| HMGA1 | Hs00852949_g1 | Thermo Fisher Scientific |
| HMOX1 | Hs01110250_m1 | Thermo Fisher Scientific |
| HOTAIR | Hs03296631_m1 | Thermo Fisher Scientific |
| ID4 | Hs02912975_g1 | Thermo Fisher Scientific |
| KIT | Hs00174029_m1 | Thermo Fisher Scientific |
| MMP7 | Hs01042796_m1 | Thermo Fisher Scientific |
| MYC | Hs00153408_m1 | Thermo Fisher Scientific |
| SFRP4 | Hs00180066_m1 | Thermo Fisher Scientific |
| SOX9 | Hs00165814_m1 | Thermo Fisher Scientific |
| SOX18 | Hs00746079_s1 | Thermo Fisher Scientific |
| TGFB1 | Hs00998133_m1 | Thermo Fisher Scientific |
| TGFB3 | Hs01086000_m1 | Thermo Fisher Scientific |
| TGFBR2 | Hs00234253_m1 | Thermo Fisher Scientific |
| 18S | Hs99999901_S1 | Thermo Fisher Scientific |
| miR182 | 002334 | Thermo Fisher Scientific |
| RNU6B | 001093 | Thermo Fisher Scientific |

Data were analyzed by using ddCt method. Results are expressed as fold changes (log 2 scale). The two-sided Fisher's exact test was used for comparison of proportions. P<0.05 was considered as statistically significant. Data were presented as Mean±SEM.

### Immunohistochemical Analysis (IHC)

Paraffin sections of breast tissues at 4 μm were used for IHC following a standard protocol for 16000 Autostainer (BioGenex, Fremont, CA, USA). BRCA1 expression was evaluated by IHC with anti-BRCA1 antibody (abcam, #ab16780). A Super Sensitive™ Polymer-HRP Detection System (BioGenex, #QD430-XAKE) was used to detect the staining. Images were acquired using Olympus DP72 microscope and analyzed with ImageScope software (Leica Biosystems).

### B. Animal Study

Study Design and r-hCG Treatment

Female Sprague Dawley rats (Taconic Biosciences Inc.) at age 55 days were treated daily via intraperitoneal injection with 100 IU/day r-hCG (OVIDREL, 250 μg/0.5 ml; 250 μg of r-hCG is equivalent to 5000 IU)) or vehicle control (phosphate buffered saline) for 21 days, with rats per group.

### Rat Mammosphere Culture

Rat mammary gland 4&5 was resected 21 days after the last r-hCG treatment. Briefly, mammary gland 4&5 was resected from Sprague Dawley rats 21 days after r-hCG ((OVIDREL, 250 μg/0.5 ml) treatment. The chopped mammary tissue was placed in 1× gentle collagenase/hyaluronidase solution (#07919, Stemcell technology) and incubated for 15 hours at 37° C. with gentle shaking. After dissociation, cell pellet was resuspended with a 1:4 mixture of ammonium chloride (NH₄Cl; #07800, Stemcell technology) and cold Hanks' Balanced Salt Solution supplemented with 2% FBS and centrifuged at 350 g for 5 minutes. The resultant organoid was sequentially resuspended in 0.25% Trypsin-EDTA for 2 minutes, 5 mg/ml Dispase I (#07913, StemCell technology) plus 0.1 mg/ml DNase I (#07900, Stemcell technology) for 2 minutes and followed by filtration through a 40 μm cell strainer to obtain single cell suspension.

To enrich mammary epithelial cells, EasySep mouse mammary stem cell enrichment kit (#19757, StemCell technology) was used to enrich mammary epithelial cells. In brief, single cell suspension at a concentration of 1×10⁸ cells/ml was prepared in Hanks' Balanced Salt Solution supplemented with 2% FBS (referred to as HF), followed by 15-minute incubation with EasySep Negative Selection Mouse Mammary Epithelial Cell Enrichment Cocktail and another 15-minute incubation with EasySep Biotin Selection Cocktail. Magnet Nanoparticles were then added in and CD45+/Ter119+, CD31+ and CD140a cells were removed by magnet selection.

Mammary epithelial cell enriched single cells were plated in 6-well ultra-low attachment plate at a density of 25,000 cells/ml in complete EpiCult-B medium (#06100, Stemcell technology) containing 10 ng/ml EGF, 10 ng/ml basic fibroblast growth factor (bFGF), 4 μg/ml Heparin and 1×Pen/Strep/Fungizon. The formation of mammosphere was checked daily under an inverted microscope. After 7 days of culture, the number of mammospheres were counted and then the mammospheres were used for other studies. Three rats from each group were used for mammosphere study.

### Microarray Analysis of Rat Mammospheres and IHC of Rat Mammary Gland

Primary mammospheres were collected by 40 μm cell strainer after 7 days of culture. Total RNA was extracted using RNAqueous Micro Scale RNA Isolation Kit (#AM1931, Invitrogen). Two hundred nanogram of total RNA per rat from three rats per group were used for the microarray hybridization using the Quick Amp Labeling Kit-one color (Agilent Technologies, Palo Alto, CA) following manufacturer's protocol. Labeled cRNAs were hybridized to Whole rat genome (4×44K) Oligo Microarrays (G4413IF, Agilent Technologies). Normalization and statistical data analysis were conducted by using limma package of Bioconductor under R environment. A cutoff of fold change of 1.5 and 2.0 and FDRp<0.05 was set to select the DEGs. IHC was performed on mammary gland tissues to validate microarray data.

Briefly, normalization and statistical data analysis were conducted by using limma package of Bioconductor under R environment. Background correction was performed using “normexp” method in the package to adjust the local median background estimates. The resulting data were then normalized by using “quantile” method whose goal is to impose to each array the same empirical distribution of intensities. The statistical analysis of normalized log 2-ratio data was carried out by applying empirical Bayes moderated t-test provided in limma software. The p values and the false discovery rate (FDR) using Benjamini-Hochberg method were calculated for every comparison. A cutoff of fold change of 1.5 and 2.0 and FDR p<0.05 was set to select the differentially expressed genes.

The functional analyses of DEGs were carried out independently for up- and down-regulated genes. To identify the gene ontology (GO) terms in the biological process category that were over-represented among the DEGs, conditional hypergeometric tests were performed in the Bioconductor GOstats package. GO terms with p<0.05 were considered enriched. Then, manually, equivalent GO terms were grouped together in larger classes of biological functions. The DEGs were also imported into Ingenuity Pathway Analysis (IPA version: 11904312) based on the Ingenuity Pathways Knowledge Base (IPKB), where each interaction in IPKB is supported by the underlying publications and structured functional annotation (Calvano et al. 2005, or world wide web at “www.ingenuity.com/”). Statistical scores were then assigned to rank the resulting networks and pathways by using Fisher's right tailed exact tests, where the significantly enriched pathways (p<0.01) were selected.

Paraffin sections of rat mammary gland at the thickness of 4 μm were used for IHC. Five rats per group were analyzed. Rabbit anti-Cd24 (#251181, ABBIOTEC) was used to detect Cd24 expression. Images were acquired with a 40× objective using Olympus DP72 microscope. Eight fields for ducts and 8 fields for lobules were randomly acquired for each mammary gland. The intensity of Cd24 in each image was evaluated and given a score of 0 to 3. A score of 0 represents no staining, 1 represents weak intensity, 2 represents moderate intensity, and 3 represents high intensity. The final scored data was analyzed by a statistician using a model that is similar to odd ratios (ORs) from logistic regression model.

### C. In Vitro Study

Cell Culture and r-hCG Treatment

Human breast epithelial cell line MCF10A with BRCA1 mutation (185delAG/+) (referred as BRCA1mut/+) and BRCA1 wild type (referred as BRCA1+/+) were purchased from Horizon Discovery, cell lines MCF10F and MCF12A were purchased from ATCC. Briefly, human breast epithelial cell lines MCF10A with BRCA1 mutation or wild type, MCF10F, and MCF12A were cultured in Dulbecco's modified Eagle medium (DMEM): F12 from Gibco containing 1.05 mM calcium, 1× antibiotic-antimycotic (#15240-062, Gibco), 20 ng/ml human EGF (#236-EG, AMGEN), 10 mg/L insulin (#15500, Sigma), 5 mg/ml hydrocortisone (#H-4001, Sigma), 100 ng/ml cholera toxin vibrio (#C-3012, Sigma), and 5% horse serum.

Cells in exponential growth phase were plated in tissue culture flasks or dishes, allowed to attach overnight, and then treated with r-hCG (OVIDREL) at 10-100 IU/ml daily for three consecutive days. Total cell lysates, nuclear extracts, and RNA were prepared at the end of 72-hour treatment. In addition, one flask of control or treated cells were maintained in normal culture media, passaged every two to three days, and used at 5, 6, or 10 days after r-hCG treatment for extracting RNA and proteins, or performing gamma irradiation study.

### Gamma Irradiation

At the end of 72 hours r-hCG treatment, and 5 or 10 days after r-hCG treatment, cells in culture dishes or on chamber slides were irradiated using the Shepherd Model 81-14R Cesium-137 irradiator that delivered gamma rays approximately 0.853 Gy/min during the period of the experiment. Cells were returned to the incubator immediately after irradiation. Cell lysates or chamber slides were collected 1 hour, 2 hours, 6 hours, or 24 hours after irradiation for Western blotting or immunofluorescence analysis.

### Western Blotting (WB) and Immunofluorescence

Cell lysates and nuclear fraction were made at different time points for WB. The band intensities of immunoblots were quantitated using Image Studio (LI-COR) or ImageJ software. One represent blot from three experiments was shown for each gene. Immunofluorescences was performed on cells cultured on chamber slides.

Cells were lysed using cold RIPA buffer (#89900, Thermo Scientific™) supplemented with protease inhibitor (#1862209, Thermo Scientific™) and phosphatase inhibitors (#P0044 and #P5726, Sigma). Nuclear fraction was extracted using NE-PER™ Nuclear and Cytoplasmic Extraction Reagents (#78833, Thermo Scientific™). Forty μg of total lysates or 30 μg of nuclear extracts were separated on NuPAGE Bis-Tris Gel (#NP0321BOX, Invitrogen) and transferred to nitrocellulose blotting membrane (#GE1060013, Amersham, GE Healthcare Life Sciences), and then probed with primary and appropriate secondary antibodies. The blots were detected using either Li-Cor Odyssey imaging system (Li-Cor Biotechnologies Corporation, Lincoln, NE) or ECL™ Western Blotting Reagents (SIGMA, St. Louis, MO) and X-ray film.

### Antibodies Used for Western Blotting

| Antibody | Catalogue# | Supplier |
|---|---|---|
| BARD1 | ab50984 | abcam |
| Beta casein | SC-30041 | Santa Cruz |
| BRCA1 | SC-6954 | Santa Cruz |
| FOXO3 | PA5-27145 | Thermo Fisher Scientific |
| GAPDH | 5174S | Cell Signaling |
| Histone H3 | 14269 | Cell Signaling |
| H3K27me3 (Tri-Methyl- | 9733S | Cell Signaling |
| Histone H3 at Lys27) | | |
| P-Histone H2A.X, Ser139 | SC-517348 | Santa Cruz |
| (γ-H2AX ) | | |
| Lamin B1 | 12586S | Cell Signaling |
| P53 | SC126 | Santa Cruz |
| SFRP4 | 15328-1-AP | Proteintech |
| SOX7 | ARP39667 | Aviva Systems Biology |
| SOX17 | LS-C14857 | LifeSpan Biosciences |
| TGFβ | 3711 | Cell Signaling |

Cells were cultured and treated on 4-well chamber slides (Millipore, Burlington, MA). At the end of treatment, media was removed and the cells were washed with TBS, followed with fixation in 10% buffered formalin, permeabilized and then blocked with 5% goat serum. Cells were stained with antibody γ-H2AX (P-Histone H2A.X, Ser139, #SC-517348, Santa Cruz) and detected with Alex Fluor® 488 goat anti-mouse antibody (#4408, Cell signaling). Nuclei were counterstained with DAPI (Thermo Fisher Scientific). Fluorescent images were captured and analyzed using Olympus BX53 fluorescent microscope with Retiga™ 2000β Fast 1934 Digital CCD Camera-Monochrome (QIMAGING Corporation, Burnaby, BC, Canada) and MetaMorph 7.7.8.0 (Molecular Devices, Sunnyvale, CA).

### MicroRNA Assay and Quantitative RT-PCR

MicroRNA and total RNA were extracted from cultured cells using miRNeasy Mini Kit (#217004, Qiagen) and AllPrep DNA/RNA Mini Kit (#80204, Qiagen). The expression of the genes of interest was evaluated with the methods described in the quantitative RT-PCR validation of clinical trial section. Data were presented as Mean±SD (n=3).

### Statistical Analysis

The Chi-square test was used when comparing γ-H2AX foci in two groups. Paired two-tailed Student's t test was used for comparing mRNA expression with and without r-hCG treatment. Student's t test was used for the comparison between two cell lines. All statistical analyses were performed using SigmaPlot 12.0 software (Systat Software Inc., San Jose, CA).

### Example 2: Use of rhCG in Women Carrying BRCA1/2 Mutations to Prevent Breast Cancer

The genomic profile of breast epithelial cells obtained from core biopsies specimens performed in 33 high-risk women treated for 90 days with OVIDREL® Prefilled Syringe (choriogonadotropin alfa) (Serono) was studied following three weekly injections of 250 μg rhCG for a total of 12 weeks. The comparison of the RNA sequence profiles before and after treatment with rhCG, both at 90 and 270 days, are of particular importance in determining the duration of the hCG effect on the transcriptomic profile.

### Selection of Participants:

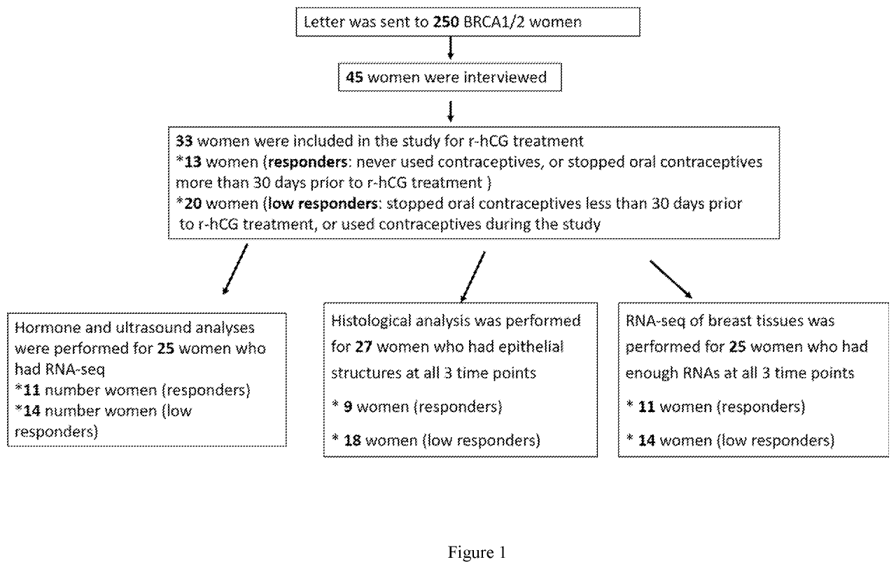

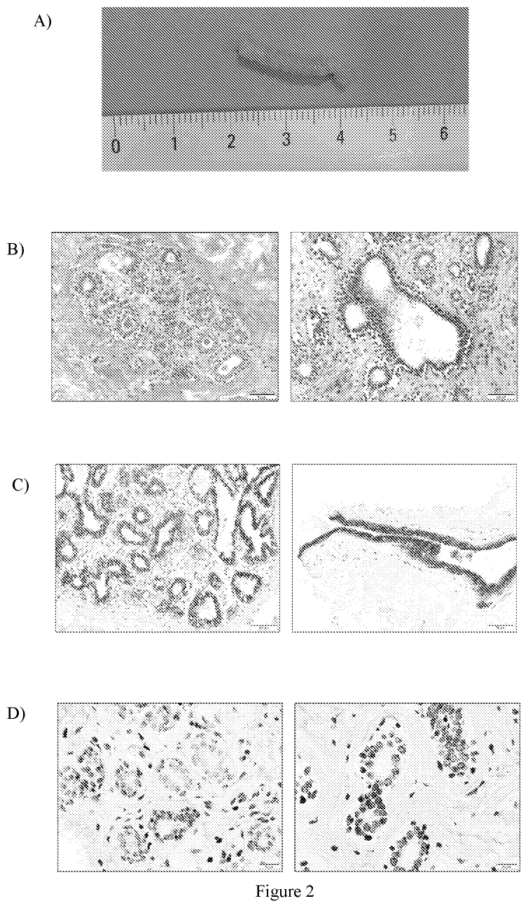

Correspondence was sent to 250 women that were proven to be BRCA1 or BRCA2 carriers, inviting them to participate in a longitudinal study involving the use of rhCG (see, FIG. 1). Thirty-three women were recruited in this prospective, longitudinal interventional study. Baseline characteristics are shown in Table 1.

TABLE 1

| Study | | | | | Contraception |
|---|---|---|---|---|---|
| number | BRCA | Mutation c1 | Mutation p1 | Age | Use |
| 101 | BRCA1 | c.5406 + 5G > A | | 25 | A |
| 102 | BRCA1 | c.5266dupC | | 23 | C |
| 103 | BRCA1 | c.2359dupG | | 21 | A |
| | | (p.Glu787Glyfs*3) | | | |
| 104 | BRCA2 | 8904del (former | Val2969fs | 24 | A |
| | | name: 9132delC) | | | |
| 105 | BRCA2 | c.4171delG | | 19 | A |
| | | (p. Glu1391Lysfs*19) | | | |
| 106 | BRCA1 | 3661G > T | Glu1221* | 25 | B (52 mg LNG |
| | | | | | over 5 years) |
| 107 | BRCA2 | 4935delA | Glu1646fs | 26 | A |
| 108 | BRCA2 | 6275_6276delTT | Leu22092Profs*7 | 18 | A |
| 109 | BRCA1 | 212 + 3A > G | | 24 | A |
| 110 | BRCA2 | 4935delA | Glu1646fs | 22 | A |
| 111 | BRCA1 | 397delC | Arg133Valfs*30 | 25 | C |
| 112 | BRCA1 | 3661G > T | Glu1221* | 21 | A |
| 113 | BRCA1 | 3661G > T | Glu1221* | 24 | B (52 mg LNG |
| | | | | | over 5 years) |
| 114 | BRCA1 | c.5194-2A > G | | 24 | A |
| 115 | BRCA1 | 134 + 3A > C | | 22 | B (13.5 mg LNG |
| | | | | | over 3 years) |
| 116 | BRCA2 | 6275_6276delTT | Leu209Profs*7 | 20 | C |
| 117 | BRCA2 | 1389_1390delAG | Val464Glyfs*3 | 22 | C |
| 118 | BRCA1 | 2359dupG | Glu787Glyfs*3 | 19 | B (13.5 mg LNG |
| | | | | | over 3 years) |
| 119 | BRCA1 | 2359dup (former | Glu787fs | 24 | C |
| | | name: 2478- | | | |
| | | 2479insG) | | | |
| 120 | BRCA1 | 3607C > T | Arg1203* | 20 | C |
| 121 | BRCA1 | 2019del (former | Glu673fs | 26 | A |
| | | name: 2138delA) | | | |
| 122 | BRCA1 | 2359dup | Glu787fs | 26 | C |
| 123 | BRCA1 | 2359dup | Glu787fs | 24 | C |
| 124 | BRCA2 | 3847_3848del | Val1283fs | 24 | A |
| | | (former name: | | | |
| | | 4075delGT) | | | |
| 125 | BRCA2 | c.5213_5216del4 | | 19 | B (EE 0.04 mg + |
| | | (p.Thr1738Ilefs*2) | | | DSG 0.15 mg) |
| 126 | BRCA1 | c.2359dupG | | 20 | B (E2 1.5 mg + |
| | | | | | Nomac 2.5 mg) |
| 127 | BRCA2 | 4171del (former | Glu1391fs | 23 | C |
| | | name: 4399delG) | | | |
| 128 | BRCA1 | 4575_4585delAGAG | Gln1525Hisfs*2 | 22 | B (19.5 mg LNG |
| | | GAGCTCA | | | over 5 years) |
| 129 | BRCA1 | 212 + 3A > G | | 25 | A |
| 130 | BRCA1 | c.3661G > T | | 22 | B (19.5 mg LNG |
| | | | | | over 5 years) |
| 131 | BRCA1 | c.3661G > T | | 18 | B (19.5 mg LNG |
| | | | | | over 5 years) |
| 132 | BRCA2 | c.662_663del | | 26 | C |
| 133 | BRCA2 | c.4576dupA | | 21 | B (etonogestrel 68 |
| | | (p.Thr1526Asnfs*3) | | | mg over 3 years) |

All women were nulliparous. The contraceptive profile consisted of 3 categories: A, B, and C (referring to Table 1). In Category A, participants did not take any hormonal medication during the study and had stopped contraception more than 30 days prior to start of study medication. In Category B, in instances where contraception containing any hormone was used, the contraceptive method is listed in this table. Three types of levonorgestrel (LNG) intrauterine systems (IUS) were used: MIRENA® (levonorgestrel-releasing IUS) (releasing 52 mg of LNG over 5 years; N=2); JAYDESS® (levonorgestrel-releasing IUS) (releasing 13.5 mg of LNG over 3 years; N=2); and KYLEENA® (levonorgestrel-releasing IUS) (releasing 19.5 mg of LNG over years; N=3). Etonogestrel (68 mg over 3 years) is an implant inserted 2 years prior to the study participation in one subject. One participant used a natural estradiol-containing oral contraceptive (1.5 mg of 17β-estradiol (E2)+2.5 mg of Nomac (nomegestrol acetate)). Another participant used an oral formulation containing ethinyl estradiol (EE) 0.04 mg combined with 0.15 mg of desogestrel (DSG). In Category C, participants did not take any hormonal medication during the study but stopped contraception less than 30 days prior to the start of study medication.

To be included in the study, the participants had to be asymptomatic, nulligravid women between 18 and 30 years of age, and carriers of the BRCA1 or BRCA2 mutation. The ECOG performance status needed to be 0 (Kornofsky 100%). Women needed to be willing to use mechanical contraceptive methods (condom, intrauterine device, abstinence). Hormonal intrauterine devices (IUD) such as the levonorgestrel (LNG)-releasing intrauterine system (LNG-IUS) that releases LNG were allowed as a contraceptive method.

Participants were excluded if they: 1) were receiving any other agents, investigational or otherwise, for the purpose of primary prevention; 2) had a history of allergic reactions attributed to compounds of similar chemical or biologic composition to rhCG preparations or one of its excipients; 3) were receiving medications that could interfere with the study protocol objectives such as prednisone, thyroid hormones, or insulin; 4) had previous treatment with follicle-stimulating hormone (FSH) for assisted reproduction; 5) had uncontrolled intercurrent illness including, but not limited to ovarian enlargement of undetermined origin, ongoing or active infection, NYHA≥class 1 congestive heart failure, unstable angina pectoris, cardiac arrhythmia, severe cognitive deficit or psychiatric illness/social situations that could make the participant unable to give informed consent or would limit compliance with study requirements; or 6) were HIV-positive, or had an infection with hepatitis B or C.

### Clinical Protocol:

Participants were asked to stop oral contraception prior to the study. The actual study was initiated during a natural cycle, if possible during the luteal phase, to avoid increased recruitment of follicles and potential overstimulation. Since no signs of hyperstimulation were observed and since it was difficult for young women to wait until a natural cycle resumed (some women did not have a regular cycle before they used oral contraceptives), it was subsequently deemed acceptable to start rhCG treatment within a week of stopping oral contraceptive use. Since some women were taking oral contraceptives because of acne or irregular bleeding, with a typical polycystic ovary syndrome image on ultrasound, the resumption of a potential ovulatory cycle was not awaited. Participants were subsequently allowed to start rhCG administration soon after stopping hormonal contraceptives. It was expected that a LNG-IUS would not interfere with the study protocol, since it was previously published that the amount of LNG in the breast epithelium was extremely low. As such, 7 women with a LNG-IUS could be included in the study without needing to remove the LNG-IUS. One woman had a long-acting reversible contraceptive implant (LARC) (68 mg of IMPLANON® (etonogestrel) over 3 years), inserted 2 years prior to the study. Another woman took a natural estrogen-containing pill during the study (1.5 mg of 17β-estradiol+2.5 mg of Nomac (nomegestrol acetate)). Another woman used an oral contraceptive (0.02 mg of ethinyl estradiol (EE)+0.15 mg of MERCILON® (desogestrel)). Since 31 women were in a stable relationship, more than 36 weeks of condom use was accepted as not being a reliable option for some participants. In the end, the study comprised 13 women who stopped using hormonal contraception more than 30 days prior to starting the rhCG medication, 10 women who started rhCG administration soon after stopping oral contraception, and 10 women who were using one form of steroidal contraception. This flexibility allowed for a 100% compliance rate in the study and avoided any unwanted pregnancies.

### SPIROTOME® Biopsy:

Blood was drawn and an ultrasound of both ovaries and the uterus was performed. If all examinations were normal, a SPIROTOME® (biopsy needle) (Bioncise, Belgium) biopsy was performed. Following the biopsy, the rhCG treatment was initiated. The study participants were taught to inject themselves with the rhCG (OVIDREL® Prefilled Syringe (choriogonadotropin alfa) (Serono)). Participants received a subcutaneous injection of 250 μg of rhCG 3 times a week (Monday, Wednesday, and Friday) for 12 weeks. The first dose of study drug was administered by a registered nurse. At that time, the nurse instructed each participant in the self-administration of the study drug by the subcutaneous route. Subsequently participants returned to receive doses 2 and 3, where they were observed by the registered nurse during the self-administration of the drug to confirm mastery of the skill and to answer any additional questions. The remainder of the drug doses were self-administered at home by the participants or by someone else trained in the procedure. All participants were seen by a study physician once a month during the treatment phase.

At inclusion, the start of the rhCG administration, and subsequently every month, blood was drawn and an ultrasound checkup of the ovaries and uterus was performed. This was carried out to exclude hyperstimulation or cyst formation. Four and 8 weeks after the final rhCG administration, blood was drawn and an ultrasound checkup of the ovaries and uterus was performed. This was carried out to assess resumption of the menstrual cycle. Since no information was available regarding prolonged rhCG administration in young women, the function of the pituitary-ovarian axis was closely monitored.

Breast tissue was obtained through a 4-mm biopsy needle using a SPIROTOME© biopsy system before, and immediately and 6 months after the 12-week treatment with rhCG. This was carried out to assess whether transcriptomic and histological changes occurring in the breast due to rhCG treatment persisted after 6 months' follow up. A rigorous follow-up protocol during and after the study was implemented to monitor the acceptance rate, procedural inconsistencies, interferences with clinical parameters, side effects, and safety of prolonged rhCG administration in these young women.

The SPIROTOME® biopsy was performed on the right lower inferior quadrant of the breast. The site was chosen to give the least esthetic impact of the small scan scar that may originate from the biopsy. An area with enough glandular tissue was selected by breast ultrasound (12-15 Hz probe, Medison, Germany). After disinfection of the skin, a disposable drape with an 8 cm round opening was attached to the biopsy area. First, a local anesthetic (0.5 mL of 1% xylocaine) was injected into the skin using a 26-gauge needle. The future trajectory of the SPIROTOME® biopsy was then anaesthetized using 10 mL of the anesthetic injected via a 22-gauge needle. A small 4 mm cut in the skin was performed using a pointed bistoury. Subsequently the SPIROTOME® trocar was inserted. The SPIROTOME® helix was gently used to remove tissue. After the removal of the first sample, a second insertion of the SPIROTOME® helix was performed through the cutting cannula/coax to remove a second tissue specimen. After the biopsy, the skin was covered with 3M Steri-Strips™. Both tissue specimens were divided into 2 parts. One fragment was placed in 70% alcohol and the other tissue fragments were stored in RNAlater. The biopsies were always obtained on Monday, Tuesday, or Wednesday so that the shipment with chemical icepack, in special containers, was carried out during the week.

### Ultrasound Monitoring:

Ultrasound examination of the endometrium, uterus, and ovaries was performed with a vaginal probe (7.5 Hz, Medison, Germany). The left and right ovaries were measured in 2 dimensions and follicles and cysts were recorded. The size of the uterus, fundal diameter, isthmus-fundal distance, and endometrial thickness and appearance (triple lining or luteal uniform appearance) were recorded. These ultrasound measurements were performed prior to the start of the rhCG treatment and every month thereafter. This was performed to exclude potential unexpected side effects of rhCG.

### Hormone Level Monitoring:

At baseline and thereafter, blood was drawn to determine estradiol, progesterone, FSH, LH, and hCG levels. Blood samples were taken before the biopsy and centrifuged at 3000 rpm for 15 minutes. The serum was stored at −80° C. Estradiol and progesterone serum levels were used to monitor the cycle. Since none of the participants had any complaints during the rhCG administration, and no signs of ovarian dysfunction were observed on ultrasound monitoring, blood was analyzed in one batch at the end of the study. The hormones and SHBG were measured by electro-chemiluminescence immunoassay (ECLIA) on the Elecsys30 and Cobas immunoassay analyzers.

### Hematoxylin & Eosin (H&E) Staining:

Breast tissues fixed in 70% ethanol were processed using a Modular Vacuum Processor (manufactured by Instrumentation Laboratory) upon receipt. Paraffin blocks were prepared using a Leica EG1160 Embedding Station. Paraffin sections at 4 μm thickness were sectioned using a Microm HM300 Microtome. The H&E staining was performed following a standard protocol.

### Immunohistochemistry (IHC):

Paraffin sections at 4 μm were stained with primary antibodies using a 16000 BioGenex Autostainer following a standard protocol. The antibodies used were as follows: purified mouse anti-E-cadherin (BD Biosciences, #610182) at a dilution of 1:200, and Tri-methyl-Histone (Lys27) (C36B11) Rabbit mAb (Cell Signaling, #9733S) at a dilution of 1:800. A Super Sensitive™ Polymer-HRP Detection System (BioGenex, #QD430-XAKE) was used to detect the staining. Tissues were counterstained with hematoxylin. The images were acquired using an Olympus DP72 microscope.

### Statistical Analysis for Hormones and Ultrasound:

Linear mixed models for the natural log-transformed hormones were fitted with a random intercept for patient (to account for the correlation between repeated measurements on the same patient) and with visit as a categorical fixed effect (the visit at week 1 before rhCG administration was taken as a reference). A mean profile plot of the estimated marginal means (on the original scale) was made. Average equivalence is concluded when the 90% confidence interval of the ratio of the means falls entirely within the range 0.80 to 1.25. Confidence intervals were computed using the profile method based on the likelihood ratio test. P-values were computed via Satterthwaite's degrees of freedom method.

In addition, linear mixed models were fitted with visit (categorical, with the visit at week 1 before rhCG administration was taken as reference group), responsiveness (low to moderate responders versus responders) and the 2-way interaction between visit and responsiveness in the fixed effect part of the model. Low to moderate responders were compared with responders at each visit at the 5% significance level. The estimated marginal means (on the original scale) were also plotted separately for low responders and responders as a function of time. For hormone levels that were below the detection limit, a value of half of the detection limit was used.

### Results:

The size of one breast biopsy specimen is shown in FIG. 2A. To assess whether the quality of breast biopsy was sufficient for histological analysis, paraffin sections were stained with H&E and evaluated under a microscope. As shown in FIG. 2B, the tissue morphology was appropriately preserved, the lobules and ducts were clearly identified by H&E staining, and the nuclear structure was also preserved in these cells. The breast tissues of BRCA1/2 carriers contain very dense stroma and fewer well defined lobules compared to the breast tissues of BRCA1/2 wild type women. The stroma-parenchyma ratio was difficult to determine, as the tissue specimen was very small. Despite this, sections containing breast parenchyma for further analysis were obtained from the majority of the specimens (27 women).

To evaluate if the proteins (antigens) in cells or tissues of these breast biopsy specimens were properly preserved for investigation using the IHC method, paraffin sections were stained with 2 antibodies: anti-E-cadherin, an epithelial cell marker expressed on the cell membrane, or anti-H3K27me3 that stains for tri-methylation at the lysine residue 27 of the histone 3 protein on cell nuclei. FIG. 2C shows that the breast epithelial cells in lobules and ducts are stained positive for E-cadherin on the cell membrane and cytoplasm, with a more intense staining on the cell membrane. The staining of H3K27me3 was located on the cell nuclei as expected (FIG. 2D). The pattern of the staining in these samples was similar to that of staining in the tissues fixed with 10% formalin, suggesting that these biopsy specimens are useful for both genetic and epigenetic studies.

### Ultrasound Changes:

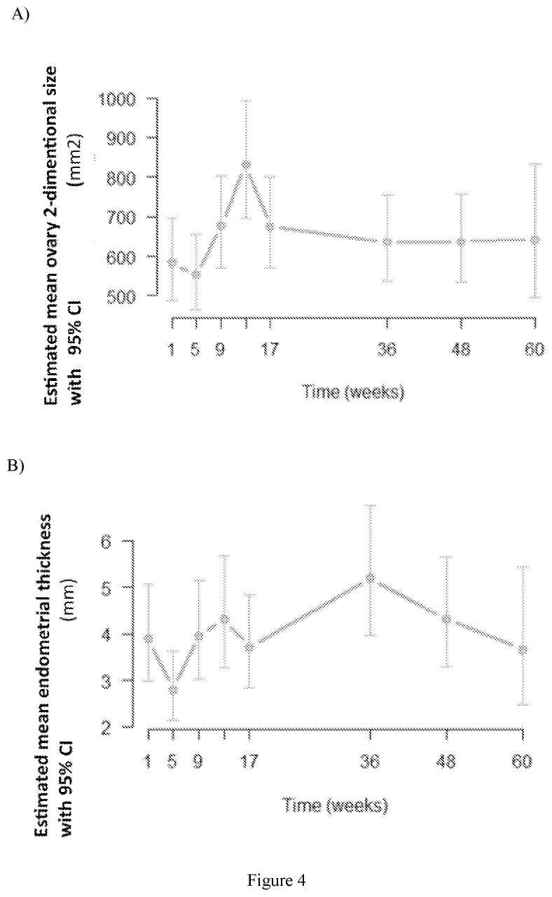

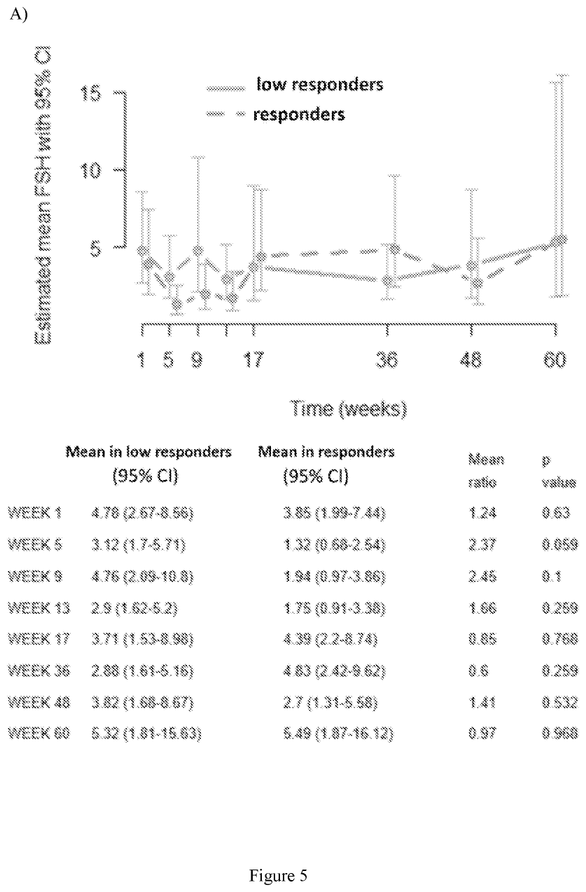

Ultrasound changes in the ovary induced by prolonged rhCG use were monitored before, during, and after the treatment. Ultrasound was performed at intake, before administration (Week 1) of the rhCG, every month during the drug administration (week 5, week 9, week 13), and 1 month after the last rhCG use (week 17). Measurements of the left and right ovary were not different from each other and were pooled. The ovaries were measured in width and length, and the 2-dimensional surface size was calculated. There was a significant, gradual increase in the size of the ovaries, from 582 (488-694) mm² at the beginning of the study, to a significantly higher surface of 831 (697-991) mm² (mean ratio 1.43 (1.19-1.71), p=0.002) at the end (week 13) of the rhCG administration. After the study was completed, the size of the ovaries remained within the values before the administration of the medication (FIG. 4A and FIG. 4B). No clinically relevant changes were observed either by ultrasound or reported subjectively by the participants during the study. No cyst formation was observed.

The changes induced by rhCG on the uterus were assessed by measuring the endometrial thickness and its appearance (triple lining, luteal appearance), the fundal diameter, and isthmus-fundal distance. There was a marginal significance of decrease in endometrial thickness from 3.9 cm (2.98-5.11) to 2.79 cm (2.13-3.66) (mean ratio 0.72 (0.54-0.95), p=0.059). The subsequent values for endometrial thickness were not different (FIGS. 4A and 4B).

### Hormonal Changes:

During the rhCG administration, there was a decrease in FSH and LH levels. FSH decreased from 3.6 (2.4-5.2) mIU/mL (reference time: T1=week 1) at the start of the study to a significantly lower value of 1.9 (1.3-2.8) mIU/mL (mean ratio 0.54 (0.35-0.83), p=0.021) at week 5. The subsequent FSH levels were not significantly different and were 2.9 (1.8-4.5) mIU/mL (mean ratio 0.8 (0.5-1.3)), and 2.7 (1.8-3.9) mIU/mL (mean ratio 0.75 (0.49-1.15)) at week 9, and week 13, respectively. The LH levels significantly decreased from 5.7 (4.3-7.7) mIU/mL at the start of the study to 1.6 (1.2-2.2) mIU/mL (mean ratio 0.28 (0.2-0.38), p<0.001) at week 5, and 3.9 (2.8-5.7) mIU/mL (mean ratio 0.69 (0.48-0.99), p=0.098) at week 9. During the last month of rhCG administration the LH normalized to 4.57 (3.37-6.21) (mean ratio 0.8 (0.58-1.11)). After the administration of the study medication, LH was not different from values at the beginning of the study.

Since FSH and LH are the drivers for follicular development, one would expect a decrease in estradiol. However, despite the decrease in FSH and LH, estradiol levels remained the same, within the normal range and not different from the initial estradiol levels. The increased hCG levels clearly compensated for the loss of gonadotropin stimulation. No significant changes were observed in the estradiol and progesterone levels.

The observed serum hCG levels clearly reflected the period of administration, with a quick elimination from the circulation at the end of the administration. The levels obtained were not associated with complaints typical for pregnancy. The levels remained between 198 (174-225) IU/L at week 5 and 161 (141-182) IU/L at week 13.

In 25 women the quality and quantity of RNA was adequate for RNA-seq analysis for all 3 time points. The response to rhCG treatment evaluated by the number of DEGs varied between study participants. The response was related to the history of contraceptive use. Whether this variation could be explained by differences in hormone levels during the study was assessed. The following differences were observed:

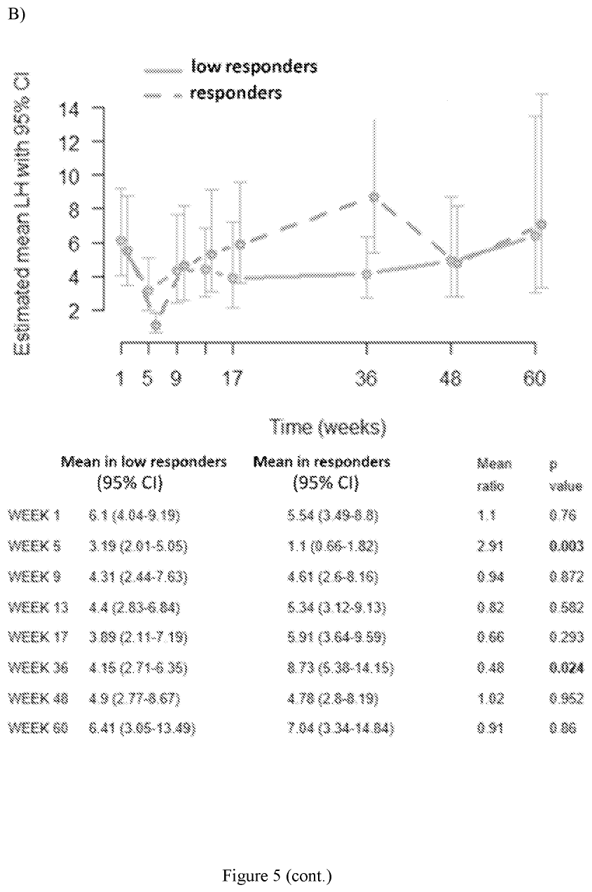

First, the responders had the lowest level at week 5 and the peak (or close to peak) at week 36 for both serum FSH (FIG. 5A) and LH (FIG. 5B); whereas the FSH and LH levels in the low responders did not vary much during the trial. There was a marginal significance for FSH at week 5 (p=0.059, mean ratio for low responders to responders=2.37) between the 2 groups. The LH was significantly different at both week 5 (p=0.003, mean ratio=2.91) and week 36 (p=0.024, mean ratio=0.48) between the 2 groups. The mean FSH levels at weeks 5, 9, and 13 (pooled analysis of hormone measurements from these 3 weeks) were significantly different (p=0.028, mean ratio low responders to responders=1.98). The mean of LH levels at weeks 5, 9, and 13 was not significantly different (p=0.204) between low responders and responders.

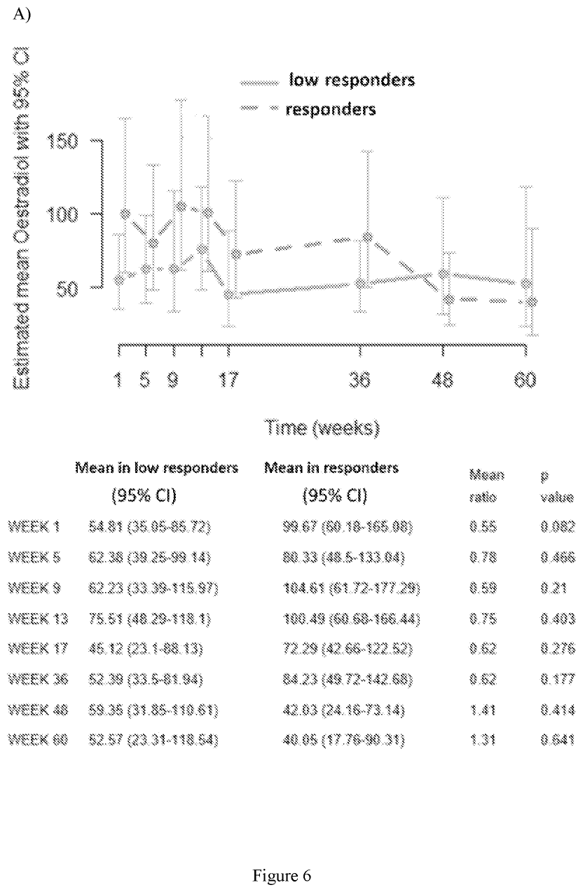

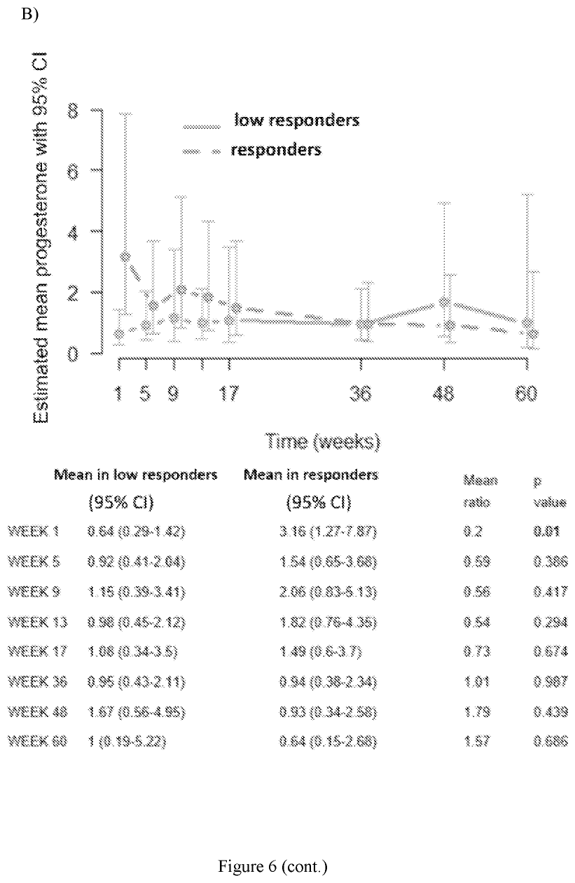

Second, responders had a higher level of estradiol (p=0.078, mean ratio=0.55) and progesterone (p=0.01, mean ratio=0.2) compared to low responders at week 1 (FIG. 6A and FIG. 6B). There was a remarkable reduction for both estradiol and progesterone in responders at week 5 and a peak at week 9, and after that time the levels of estradiol and progesterone decreased. Generally, the mean levels of estradiol and progesterone in the responders were always higher than those in the low responders at each time point during the first 36 weeks of the trial. After 36 weeks, the levels of estradiol and progesterone were tendentiously lower in the responders. Although the changes after week 1 are not significant due to the small population size, the tendencies of circulating estradiol and progesterone were different between the 2 groups, responders and low responders, at each time measurement.

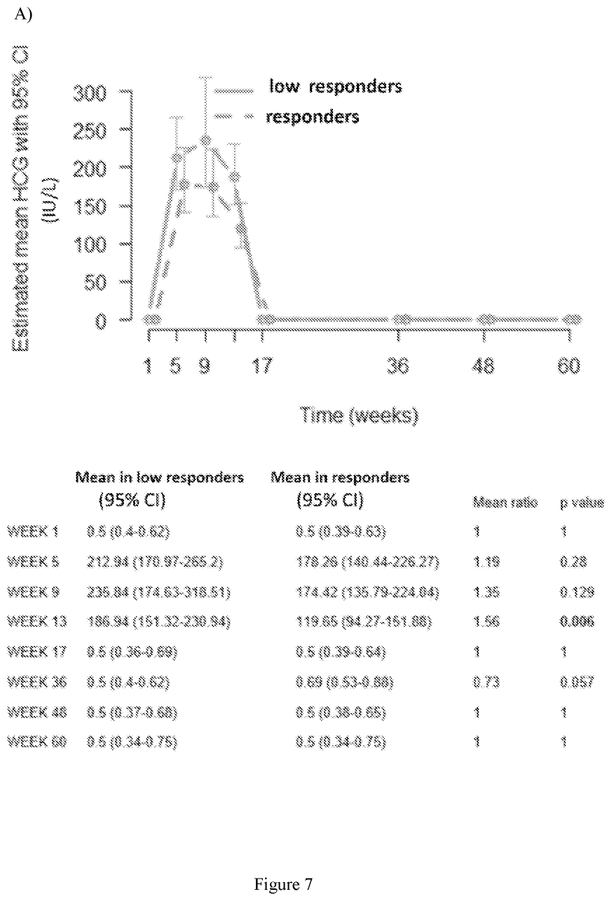

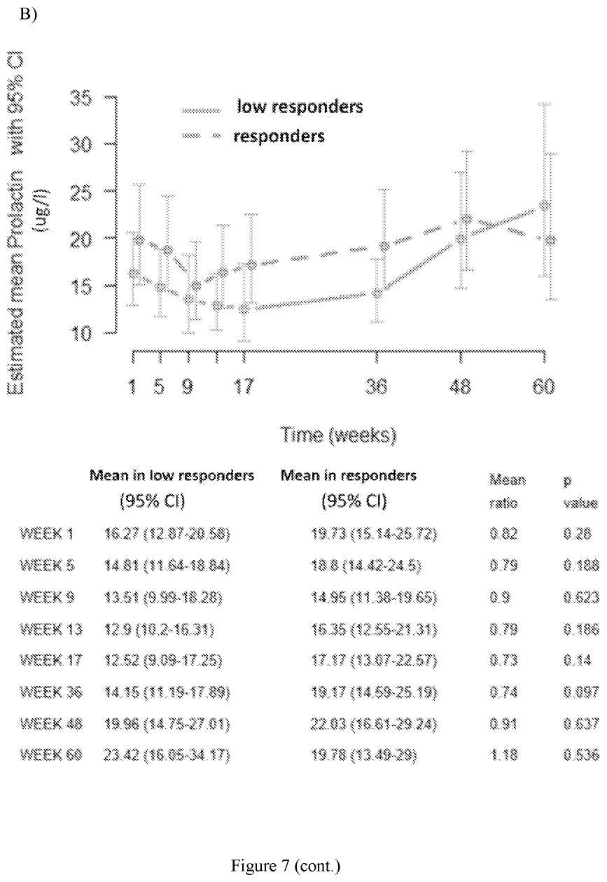

Third, mean hCG level was 206 (180-237) IU/L at week 5, 9, 13 in the low responders, it was significantly (P<0.005) higher than the mean value 154 (134-178) IU/L in the responders (mean ratio low responders to responders=1.34) (FIG. 7A). The levels of prolactin were not significantly different between groups, not even when they were pooled (FIG. 7B). The level of prolactin in lower responders showed a trend of decrease at week 36 (T3) compared to the prolactin level in responders (p=0.097).

Taken together, the levels of FSH and LH decreased significantly at week 5 and reached peak levels at week 36 for the responders, with the more decreased levels maintained during the first 13 weeks (period of rhCG administration) compared to those of the low responders; after 13 weeks, when rhCG treatment stopped, the levels of FSH and LH in the responders started to increase, causing the surge of both of these hormones at week 36 (time point 3). The serum levels of estradiol and progesterone were higher in the responders during the time of rhCG administration and were maintained up to 36 weeks compared to the low responders. The hormone levels did not change much, and showed only a very small fluctuation in the low responders.

### Discussion:

The results showed that a history of using hormonal contraceptives affects the response of the breast to rhCG treatment. This is a very important observation because the breast is a hormone-responsive organ. The lower serum hCG level observed in responders might suggest a higher binding of rhCG in target organs. Consistently, the serum estrogen and progesterone levels were relatively higher in responders during 36 weeks of the study, indicating a higher hCG response. High circulating concentrations of estrogen and progesterone increase prolactin during pregnancy. In the present study, the serum prolactin levels are in agreement with serum estrogen and progesterone levels. Interference from medication, hormonal status and hCG can act in 2 ways. The influence of hCG on clinical and endocrine parameters seems minimal and even absent. The effect of clinical parameters on hCG efficiency is unexpected. Hormonal use seems to have a paramount effect on molecular biology parameters. This observation is the first of its kind and subsequent prevention studies should take into account stratification according to contraception techniques and wash-out periods.

Initially, administration of rhCG was started during the luteal phase. Since recruitment and maturation of one follicle had taken place, it was considered that this would be a safe option to avoid multiple follicle recruitment. This was done to avoid potential hyperstimulation. As evidenced by laboratory tests and ultrasound monitoring, prolonged administration was safe, and no significant increase in estradiol levels were observed. The surface of the ovaries was used as a parameter of the size of the ovaries, reflecting the degree of ovarian stimulation. Again, no significant increase in ovarian surface was observed. Since in the initial participants no signs of OHSS were observed, women were allowed to start rhCG soon after stopping hormonal contraception, not requiring them to start during the luteal phase. The 7 participants having a hormonal LNG-IUS were not required to have it removed prior to the study. The low amount of LNG in the breast was not believed to interfere with the study medication. Surprisingly, these women (contraception group) had different responses, with a delay and a significant reduction in DEGs.

The results show the clear difference between women exposed and not exposed to hormonal contraceptives, especially less than 30 days prior to starting rhCG treatment. In this study, there was an obvious distinction in hormonal responses to rhCG therapy between the 2 groups. Specifically, the responders had lower levels of FSH and LH during the time of rhCG administration, and both FSH and LH had a surge in responders at 6 months after the last injection of rhCG.

The administration of rhCG resulted in a significant reduction of LH and FSH levels. The expected reduction in estradiol, due to the decrease in gonadotropins, was not observed. The rhCG compensated for the decrease in stimulation from the reduced gonadotropin levels.

This study demonstrates for the first time that prolonged use of rhCG in young BRCA1/2 mutation carrier women for breast cancer prevention is feasible and safe, and the breast tissue biopsy samples collected before and after rhCG treatment are of good quality for RNA and protein analysis. RNA-sequencing analysis showed that rhCG treatment had a remarkable effect on the gene expression profile of breast tissues from BRCA1/2 carrier women who did not use any hormonal contraceptives, whereas the use of contraceptives during the study delayed the response, and significantly reduced the number of DEGs.

Given these results, rhCG preventive therapy is indicated for nulligravid women carrying BRCA1/2 deleterious mutation without any prior exposure to the hormonal contraceptives both per os or in uterine device in at least 30 days. There is a remarkable response to rhCG therapy on the gene expression profile of breast tissues from BRCA1/2 carriers who did not use any contraception before or during the trial or ones that stopped using oral contraceptives more than 30 days before the trial or used the cooper intra uterine device (IUD), whereas ones exposed to the oral contraceptives or hormonal IUDs show no- or delayed- and low-response to hCG in the trial. This study is the first report demonstrating the effect of rhCG on the gene expression of breast tissues of nulligravid women carrying BRCA1/2 deleterious mutation and unexposed to hormonal contraceptives in at least 30 days prior to initiation of rhCG treatment.

### Example 3: Genomic Signature of the Breast Induced by rhCG in Women Carrying BRCA1/2 Mutation

To address how rhCG induces BRAC1 expression in the breasts of BRCA1/2 mutation carriers, and to characterize the transcriptomic profile of breasts from these women before and after rhCG treatment, RNA-sequencing (RNA-seq) analysis was performed. Breast tissue biopsy fragments in RNAlater RNA Stabilization Reagent were immediately stored in a freezer at −80° C. upon receiving. Total RNA was extracted within a month after all samples were received using the RNeasy Lipid Tissue Mini kit (Qiagen, US) according to the manufacturer's protocol. The RNA quality was measured by a Nanodrop™—Nd-1000 Spectrophotometer (Thermo Fisher Scientific, US) and integrity was evaluated using a 2100 Bioanalyzer Instrument (Agilent Technologies, US) with an RNA 6000 Pico kit (Agilent Technologies, US) according to the manufacturer's protocol. RNA samples with an RNA integrity number (RIN) less than 4.8 were discarded. Library construction was performed using PE100 strand-specific library preparation for eukaryote (BGI, CA, US) to generate DNA nanoball (DNB), which had more than 300 copies of one molecule. The DNBs were loaded into the patterned nanoarray and pair end 100 bases reads were generated by combinatorial Probe-Anchor Synthesis (cPAS) on the BGISEQ-500 platform (BGI, CA, US) with more than 60 million reads delivered to each of the samples. The library construction and sequencing were carried out by the BGI Company in Hong Kong.

All the raw sequences were quality checked using FastQC (Babraham Institute, USA) prior to alignment. The raw reads were quality filtered to remove low-quality reads using Genomic Workbench version 12.0 (USA). The cleaned reads were used for mapping against the Homo_sapiens. GRCh38 reference genomes (Esemble GRCh38/hg38) using CLC Genomics Workbench version 12.0.3 (Qiagen, US). In total, there were 166 files sequenced with each containing from 128-199 million reads. The mapping rate ranged from approximately 98% to 99% for all the samples. For analyses, only the reads aligned to 23 pairs of human chromosomes were considered. To estimate the expression level, the number of exon reads mapped per kilobase per million mapped reads, RPKM, for each gene was measured using CLC Genomics Workbench version 12.0.3 (Qiagen, US). Each gene was modeled by a separate Generalized Linear Model (GLM). The Robinson and Smyth's Exact Test implemented in the CLC Genomics Workbench version 12.0.3 (Qiagen, US), which assumes a Negative Binomial distribution of the data and takes into account the overdispersion caused by biological variability, was used to compare expression levels between each time point for treated group and controls. Fold changes were calculated from the GLM, which corrects for differences in library size between the samples. A false discovery rate (FDR)-adjusted p-value of (FDRp)≤0.05 was chosen to indicate statistical significance. The genes with absolute fold change (FC) larger than 1.5 and with an FDR p less than 0.05 were considered as differentially expressed genes (DEGs).

Analysis of differential expression between 2 time points involved adjustment for multiple testing in terms of controlling the false discovery rate (FDR). Using R version 3.4.4 package RNASeqPower34, with the RNA sequencing to an average of 11× depth for all reads of 100 base pair length in paired end and an effect size of 1 (in terms of log 2 ratios, an effect size of 1 corresponds to a 2-fold change difference between any 2 time points being compared) between any 2 time points, at a significance level of the false discovery rate of 0.05, the required sample size of approximately 11 allows the differential expression analysis of RNA sequencing of 90% power. A significance level of 0.05 results in 100 false discoveries per 2000 non-differentially expressed genes. Due to the paired nature of the comparisons, 11 participants are required for each time point. Taking into account the fact that breast samples would not always yield enough material for RNA analysis in each biopsy and that study participants were needed where all 3 biopsies could be compared, 30 women were projected to be included. Because some participants were related to each other, and it was not desired to choose amongst them for inclusion in the study, three additional women were included to end up with a total of 33 women participating in the trial. Data were imported into R version 3.4.4 & 3.5.3 and visualized with R packages for plots, diagrams and graphs.

Transcriptomic Changes: To investigate the transcriptomic changes of the breast tissue in these women before and after receiving rhCG, RNA-seq was performed for 83 breast RNA samples with good quality from 25 women using the BGISEQ-500 platform. The sequencing in paired-end 100-bp reads were generated from 128-199 million reads per sample. To ensure the quality of the reads for RNA-seq analysis, all raw reads were checked for quality using FastQC version 0.11.5 and the aggregated plots and report were generated by MultiQC (data not shown). The clean reads collected after low quality read removal were aligned against the human genome GRCh38. The total mapping rate ranged from 98-99%, and a range of 92-93% of total reads per sample were mapped in pairs to the reference genome.

To determine the difference in gene expression levels of the breast tissue prior to and after rhCG therapy, paired 2-group comparisons were conducted between the mapping results of breast tissue in women at different time points of treatment against the baseline, before receiving rhCG, using the CLC Genomics Workbench 12.0.3 with the RPKM values. The threshold p-value was determined according to the false discovery rate (FDR). In this study, genes that were considered differentially regulated met the following criteria: FDR p-value ≤0.05 and absolute fold change was ≥1.5.

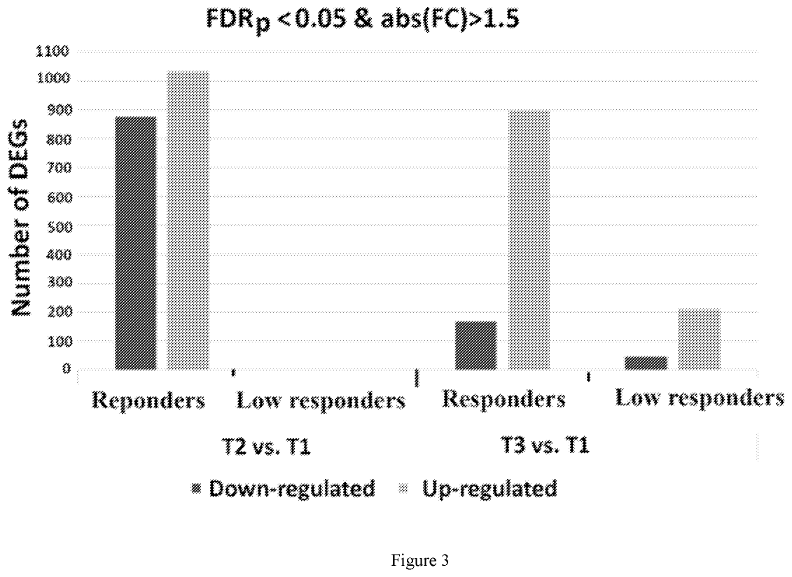

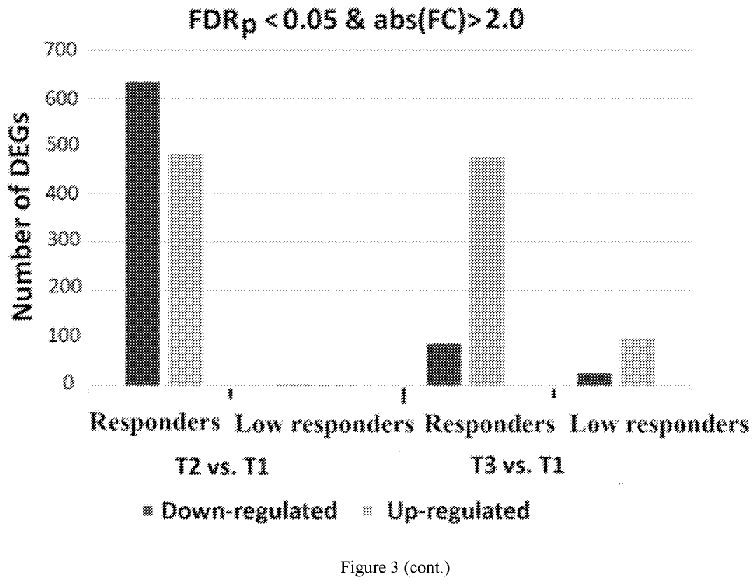

Since the response to rhCG treatment evaluated by RNA-seq varied between the study participants, the data was re-analyzed according to hormonal contraceptive use during the study. Among these 25 patients, there were 11 women who did not use contraceptives during the hCG trial or who stopped oral contraceptives more than 30 days prior to the trial (except one case using a copper IUD before, during and after the trial) and 14 women using oral contraceptives or a hormonal IUD during the trial or stopping the pills less than 30 days prior to the trial (FIG. 1). A strong difference was observed between the 2 groups, with and without contraceptive use, in the response to the rhCG at both T2 and T3 versus baseline, T1. That was clearly reflected in FIG. 3 showing the DEGs at the cutoff fold change (FC) of 1.5 and 2, respectively, with 1907 DEGs (1032 up, 875 down) at T2 vs. T1 and 1065 DEGs (897 up, 168 down) at T3 vs. T1 for the women not using contraceptives (named as responders) while there was almost no response at T2 vs. T1 and a small number of DEGs, 260 (214 up, 46 down) at T3 vs. T1 for the group of 14 women using pills or an IUD during, or stopping the pills prior to, the trial (named as low responders). Notably, the number of DEGs with the FC of 2 accounts for about half of the total number of genes with significant expression changes.

In summary, both at the end of rhCG treatment and 6 months later, rhCG has a remarkable effect on the gene expression profile of breast tissues from BRCA1/2 carrier women who did not use any hormonal contraceptives, whereas the use of a hormonal contraceptive caused an interference of hCG's effects on the gene expression response of breast tissue, delayed the responses until 6 months after treatment, and dramatically reduced the number of DEGs compared to that observed in women without hormonal contraceptive use.

The effect of rhCG on the transcriptomic profile analysis of the group of 11 women without contraceptives was the next focus. To visualize the significance and level of the changes of gene expression, genes were ranked by the log 10 FDR-adjusted-p value (log 10(pvalue)) and plotted them against the log 2 fold change (log 2FC) for each pairwise comparison in each volcano plot using R version 3.5.3. Volcano plots (data not shown) intuitively exhibited the distribution of total genes and DEGs of breast tissues at day 90 days (time point 2) and 270 days (time point 3) versus baseline before rhCG injection (time point 1).

To observe the changes of gene expression in breast tissues of 11 women without contraceptive use upon the treatment of rhCG, a heatmap was constructed on normalized gene read counts of 2135 DEGs at three time points, 01: before treatment, baseline; 02: after treatment, 3 months from baseline; 03: 6 months post treatment, 9 months from baseline (data not shown). The heatmap showed a persistent change in gene expression in breasts of these responders from right after rhCG termination to 6 months later, with some DEGs, which are more significantly different in T2 only or in T3 only compared to the baseline breasts before therapy, and some DEGs, which are consistently significantly different at both of T2 and T3 compared to that of baseline.

To identify the biological process and Reactome pathways related to DEGs of breast tissues among these women, DEGs induced by rhCG were used for the analysis using DAVID tool and Shiny application in β version 3.5.3. Significant groups of gene ontology enrichment were determined using Benjamini-Hochberg correction with cut-off levels of p<0.05. Persistently, rhCG majorly affected cellular developmental process, cell differentiation, proliferation and adhesion, MAPK/ERK1-2 cascade and G protein-coupled receptor (GPCR) signaling at both of time point 2 and 3, while apoptotic process genes were increased from 18 upregulated DEGs at time point 2 to 118 DEGs at time point 3 (data not shown). DEGs induced by rhCG at time point 2 showed the activation of CGMP mediated signaling. A great number of genes related to cell death and immune response were also observed at time point 3 (data not shown). For reactome pathway (data not shown), collagen formation, extracellular matrix organization, glycosaminoglycan biosynthesis and MAPK signaling were consistently upregulated and maintained at both time point 2 and 3. Signaling by GPCR (Reactome R-HAS-372790) was upregulated at time point 2 and GPCR ligand binding (Reactome R-HAS-500792) was upregulated at time 3, suggesting the activation of Leuteinizing hormone/choriogonadotrpin receptor.

Additionally, a great number of DEGs were identified to be associated with DNA repair, chromatin remodeling and organization at both time point 2 and 3 (data not shown). These DEGs also mainly affected DNA-templated transcription, regulation of RNA metabolic process and gene expression, cell differentiation, histone modification, cell cycle, immune response, apoptosis, double strand break repair, DNA replication, cell response to DNA damage and production of tumor necrosis factor at time-point 2 (Table 2 and Table 3). For instance, it was found that PADI2, MYC, and SOX9 were down-regulated while PADI3 was upregulated in breast tissues of BRCA1/2 mutation carrier women.

TABLE 2

| Enrichment | Genes | | |
|---|---|---|---|
| FDR | in list | Function | Up-regulated genes |
| 6.05E−13 | 14 | Chromatin organization | JAK2 PADI3 AICDA PRKCD |
| | | | PRDM6 MECOM SATB2 TWIST1 |
| | | | LOXL2 MAP3K12 JDP2 IGF2 |
| | | | GATA2 TAL1 |
| 3.66E−05 | 14 | Regulation of RNA | PRDM6 TAL1 MECOM AICDA |
| | | metabolic process | TWIST1 LOXL2 JDP2 GATA2 JAK2 |
| | | | SATB2 ZNF385A IGF2 MAP3K12 |
| | | | PRKCD |
| 6.50E−05 | 14 | Cellular protein | JAK2 LOXL2 MAP3K12 PADI3 |
| | | modification process | PRKCD IGF2 ISG15 PRDM6 |
| | | | MECOM TWIST1 PPP2R1B JDP2 |
| | | | TAL1 GATA2 |
| 0.000149676 | 14 | Regulation of gene | PRDM6 ZNF385A TAL1 MECOM |
| | | expression | AICDA TWIST1 LOXL2 JDP2 |
| | | | GATA2 JAK2 SATB2 IGF2 |
| | | | MAP3K12 PRKCD |
| 0.0001156 | 13 | Transcription, DNA- | PRDM6 TAL1 MECOM AICDA |
| | | templated | TWIST1 LOXL2 JDP2 GATA2 JAK2 |
| | | | SATB2 ZNF385A IGF2 MAP3K12 |
| 0.000123623 | 13 | Nucleic acid-templated | PRDM6 TAL1 MECOM AICDA |
| | | transcription | TWIST1 LOXL2 JDP2 GATA2 JAK2 |
| | | | SATB2 ZNF385A IGF2 MAP3K12 |
| 0.000129803 | 13 | RNA biosynthetic process | PRDM6 TAL1 MECOM AICDA |
| | | | TWIST1 LOXL2 JDP2 GATA2 JAK2 |
| | | | SATB2 ZNF385A IGF2 MAP3K12 |
| 0.000268799 | 13 | Cell differentiation | PRDM6 JAK2 LOXL2 TAL1 GATA2 |
| | | | MECOM SATB2 TWIST1 JDP2 |
| | | | ZNF385A IGF2 ISG15 AICDA |
| 3.86E−11 | 11 | Covalent chromatin | JAK2 PADI3 AICDA PRKCD |
| | | modification | PRDM6 MECOM TWIST1 MAP3K12 |
| | | | JDP2 IGF2 GATA2 |
| 6.72E−07 | 11 | Peptidyl-amino acid | JAK2 LOXL2 PADI3 PRKCD |
| | | modification | PRDM6 MECOM TWIST1 MAP3K12 |
| | | | TAL1 IGF2 GATA2 |
| 8.77E−10 | 10 | Histone modification | JAK2 PADI3 PRKCD PRDM6 |
| | | | MECOM TWIST1 MAP3K12 JDP2 |
| | | | IGF2 GATA2 |
| 0.000130111 | 10 | Cellular response to stress | RECQL MAP3K12 BIVM-ERCC5 |
| | | | SAMHD1 PRKCD JAK2 TWIST1 |
| | | | ZNF385A MECOM ISG15 |
| 0.000991655 | 10 | Transcription by RNA | TAL1 TWIST1 LOXL2 JDP2 GATA2 |
| | | polymerase II | PRDM6 MECOM SATB2 ZNF385A |
| | | | IGF2 |
| 0.002902235 | 10 | Immune system process | JAK2 SAMHD1 AICDA TAL1 |
| | | | PRKCD IGF2 GATA2 ISG15 |
| | | | ZNF385A MECOM |
| 0.002073991 | 8 | Apoptotic process | PRKCD MECOM JAK2 TWIST1 |
| | | | MAP3K12 ZNF385A PPP2R1B |
| | | | GATA2 |
| 0.002409577 | 8 | Cell proliferation | IGF2 GATA2 JAK2 TWIST1 TAL1 |
| | | | PRKCD LOXL2 MECOM |
| 0.00297636 | 8 | Programmed cell death | PRKCD MECOM JAK2 TWIST1 |
| | | | MAP3K12 ZNF385A PPP2R1B |
| | | | GATA2 |
| 0.004175164 | 8 | Cell death | PRKCD MECOM JAK2 TWIST1 |
| | | | MAP3K12 ZNF385A PPP2R1B |
| | | | GATA2 |
| 0.000175399 | 7 | Cellular response to DNA | RECQL BIVM-ERCC5 SAMHD1 |
| | | damage stimulus | ZNF385A TWIST1 PRKCD ISG15 |
| 0.040292604 | 6 | Immune response | SAMHD1 ISG15 JAK2 AICDA |
| | | | PRKCD GATA2 |
| 0.00111224 | 5 | DNA repair | RECQL BIVM-ERCC5 SAMHD1 |
| | | | TWIST1 ISG15 |
| 0.006917173 | 5 | MAPK cascade | MAP3K12 IGF2 JAK2 MECOM |
| | | | PRKCD |
| 0.008410491 | 4 | Apoptotic signaling | JAK2 PRKCD PPP2R1B ZNF385A |
| | | pathway | |
| 0.006032341 | 3 | Double-strand break | RECQL SAMHD1 TWIST1 |
| | | repair | |
| 0.007811241 | 3 | DNA recombination | RECQL SAMHD1 AICDA |
| 0.003456411 | 2 | Intrinsic apoptotic | JAK2 PRKCD |
| | | signaling pathway in | |
| | | response to oxidative | |
| | | stress | |
| 0.00503772 | 2 | Cell differentiation in | TAL1 GATA2 |
| | | spinal cord | |
| 0.00844616 | 2 | Cell death in response to | JAK2 PRKCD |
| | | oxidative stress | |
| 0.01315764 | 2 | DNA damage response, | TWIST1 ZNF385A |
| | | signal transduction by p53 | |
| | | class mediator | |
| 0.013236769 | 2 | Epithelial cell apoptotic | JAK2 GATA2 |
| | | process | |
| 0.015042133 | 2 | Double-strand break | RECQL SAMHD1 |
| | | repair via homologous | |
| | | recombination | |
| 0.016449391 | 2 | Signal transduction in | TWIST1 ZNF385A |
| | | response to DNA damage | |
| 0.021929918 | 2 | Tumor necrosis factor | TWIST1 JAK2 |
| | | production | |
| 0.031784677 | 2 | JNK cascade | MAP3K12 MECOM |
| 0.03383676 | 2 | Extrinsic apoptotic | PPP2R1B JAK2 |
| | | signaling pathway | |
| 0.042984481 | 2 | DNA replication | SAMHD1 AICDA |
| 0.04464424 | 2 | Signal transduction by | TWIST1 ZNF385A |
| | | p53 class mediator | |

TABLE 3

| Enrichment | Genes | | |
|---|---|---|---|
| FDR | in list | Function | Down-regulated genes |
| 0.000170893 | 3 | Base-excision repair | HMGA1 LIG1 RPS3 |
| 2.03E−05 | 11 | Cell cycle | LIG1 MYC RBBP8 GATA3 BRDT |
| | | | FANCD2 RPS3 CHAF1B CENPV SOX9 |
| | | | NPM2 |
| 0.048464103 | 2 | Cell cycle G1/S phase | RBBP8 MYC |
| | | transition | |
| 0.042588917 | 3 | Cell cycle phase | RBBP8 NPM2 MYC |
| | | transition | |
| 8.48E−05 | 9 | Cell cycle process | LIG1 MYC RBBP8 BRDT FANCD2 |
| | | | SOX9 RPS3 NPM2 CENPV |
| 0.001147597 | 2 | Cell proliferation | GATA3 MYC |
| 2.09E−05 | 5 | Chromatin assembly | CHAF1B SOX9 HMGA1 IPO4 CENPV |
| 2.41E−06 | 6 | Chromatin assembly | CHAF1B PADI2 SOX9 HMGA1 IPO4 |
| | | or disassembly | CENPV |
| 0.001438264 | 2 | Chromatin | PADI2 HMGA1 |
| | | disassembly | |
| 7.56E−13 | 14 | Chromatin | FBL PADI2 NPM2 CHAF1B RPS6KA5 |
| | | organization | SOX9 MYC HMGA1 IPO4 PABPC1L |
| | | | GATA3 BRDT L3MBTL4 CENPV |
| 1.85E−11 | 9 | Chromatin | NPM2 SOX9 MYC HMGA1 PABPC1L |
| | | remodeling | GATA3 BRDT PADI2 CENPV |
| 0.005266041 | 4 | Covalent chromatin | FBL PADI2 RPS6KA5 GATA3 |
| | | modification | |
| 4.38E−05 | 5 | DNA packaging | CHAF1B SOX9 HMGA1 IPO4 CENPV |
| 0.009743367 | 3 | DNA recombination | RBBP8 HELB LIG1 |
| 2.43E−07 | 9 | DNA repair | RBBP8 HELB HMGA1 FANCD2 RPS3 |
| | | | LIG1 CHAF1B CHRNA4 NPAS2 |
| 1.28E−05 | 6 | DNA replication | LIG1 NPM2 HELB CHAF1B HMGA1 |
| | | | RBBP8 |
| 0.002344243 | 2 | DNA replication- | CHAF1B IPO4 |
| | | dependent | |
| | | nucleosome assembly | |
| 0.002344243 | 2 | DNA replication- | CHAF1B IPO4 |
| | | dependent | |
| | | nucleosome | |
| | | organization | |
| 0.002344243 | 3 | DNA-dependent | LIG1 HELB HMGA1 |
| | | DNA replication | |
| 0.017809608 | 3 | Epithelial cell | GATA3 MYC SOX9 |
| | | proliferation | |
| 0.045762586 | 2 | G1/S transition of | RBBP8 MYC |
| | | mitotic cell cycle | |
| 0.00057003 | 2 | Heterochromatin | HMGA1 CENPV |
| | | assembly | |
| 0.001081081 | 2 | Heterochromatin | HMGA1 CENPV |
| | | organization | |
| 0.004830418 | 4 | Histone modification | FBL PADI2 RPS6KA5 GATA3 |
| 0.012853297 | 9 | Immune system | GATA3 PADI2 CHRNA4 SOX9 MYC |
| | | process | FANCD2 ARID5A RPS3 RPS6KA5 |
| 0.02021832 | 2 | Meiosis I cell cycle | BRDT FANCD2 |
| | | process | |
| 0.000919321 | 4 | Meiotic cell cycle | RBBP8 BRDT FANCD2 NPM2 |
| 0.004374898 | 3 | Meiotic cell cycle | BRDT FANCD2 NPM2 |
| | | process | |
| 0.004174859 | 2 | Mesenchymal cell | SOX9 MYC |
| | | proliferation | |
| 0.034874549 | 4 | Mitotic cell cycle | LIG1 RBBP8 MYC NPM2 |
| 0.037087423 | 3 | Mitotic cell cycle | RBBP8 NPM2 MYC |
| | | phase transition | |
| 0.025368318 | 4 | Mitotic cell cycle | LIG1 RBBP8 NPM2 MYC |
| | | process | |
| 0.032679565 | 2 | Notch signaling | SOX9 MYC |
| | | pathway | |
| 0.000575885 | 8 | Reproduction | SOX9 PABPC1L RBBP8 GATA3 MYC |
| | | | BRDT FANCD2 NPM2 |
| 0.030266638 | 3 | T cell activation | GATA3 FANCD2 RPS3 |
| 0.044520145 | 2 | T cell differentiation | GATA3 FANCD2 |
| 0.04817694 | 2 | T cell receptor | GATA3 RPS3 |
| | | signaling pathway | |
| 0.015910173 | 8 | Transcription by | SOX9 ARID5A GATA3 MYC NPAS2 |
| | | RNA polymerase II | RPS6KA5 HMGA1 RBBP8 |
| 4.93E−05 | 14 | Transcription, DNA- | SOX9 HMGA1 ARID5A RPS6KA5 |
| | | templated | GATA3 MYC NPAS2 PABPC1L BRDT |
| | | | FANCD2 RPS3 L3MBTL4 PADI2 RBBP8 |
| 0.000054708 | 14 | Nucleic acid- | SOX9 HMGA1 ARID5A RPS6KA5 |
| | | templated | GATA3 MYC NPAS2 PABPC1L RBBP8 |
| | | transcription | BRDT FANCD2 RPS3 L3MBTL4 PADI2 |
| 0.000000000 | 14 | Chromatin | FBL PADI2 NPM2 CHAF1B RPS6KA5 |
| | | organization | SOX9 MYC HMGA1 IPO4 PABPC1L |
| | | | GATA3 BRDT L3MBTL4 CENPV |
| 0.000037575 | 14 | Regulation of RNA | SOX9 HMGA1 ARID5A RPS6KA5 |
| | | biosynthetic process | GATA3 MYC NPAS2 PABPC1L RBBP8 |
| | | | BRDT FANCD2 RPS3 L3MBTL4 PADI2 |
| 0.000339840 | 14 | Regulation of gene | SOX9 HMGA1 ARID5A RPS6KA5 |
| | | expression | GATA3 MYC RPS3 NPAS2 PABPC1L |
| | | | BRDT FANCD2 L3MBTL4 PADI2 |
| | | | RBBP8 |
| 0.000049300 | 14 | Transcription, DNA- | SOX9 HMGA1 ARID5A RPS6KA5 |
| | | templated | GATA3 MYC NPAS2 PABPC1L BRDT |
| | | | FANCD2 RPS3 L3MBTL4 PADI2 RBBP8 |
| 7.56E−13 | 14 | Chromatin | FBL PADI2 NPM2 CHAF1B RPS6KA5 |
| | | organization | SOX9 MYC HMGA1 IPO4 PABPC1L |
| | | | GATA3 BRDT L3MBTL4 CENPV |
| 4.93E−05 | 14 | Transcription, DNA- | SOX9 HMGA1 ARID5A RPS6KA5 |
| | | templated | GATA3 MYC NPAS2 PABPC1L BRDT |
| | | | FANCD2 RPS3 L3MBTL4 PADI2 RBBP8 |
| 5.47E−05 | 14 | Nucleic acid- | SOX9 HMGA1 ARID5A RPS6KA5 |
| | | templated | GATA3 MYC NPAS2 PABPC1L RBBP8 |
| | | transcription | BRDT FANCD2 RPS3 L3MBTL4 PADI2 |
| 5.70E−07 | 10 | Cellular response to | RBBP8 HELB HMGA1 FANCD2 RPS3 |
| | | DNA damage | LIG1 MYC CHAF1B NPAS2 CHRNA4 |
| | | stimulus | |
| 0.000249389 | 10 | Cellular response to | RBBP8 HELB HMGA1 FANCD2 RPS3 |
| | | stress | LIG1 MYC CHAF1B NPAS2 CHRNA4 |
| 0.034608547 | 9 | Cell differentiation | FBL GATA3 SOX9 PABPC1L MYC |
| | | | BRDT FANCD2 NPM2 RPS6KA5 |
| 0.000084800 | 9 | Cell cycle process | LIG1 MYC RBBP8 BRDT FANCD2 |
| | | | SOX9 RPS3 NPM2 CENPV |
| 0.000000000 | 9 | Chromatin | NPM2 SOX9 MYC HMGA1 PABPC1L |
| | | remodeling | GATA3 BRDT PADI2 CENPV |
| 0.000012800 | 6 | DNA replication | LIG1 NPM2 HELB CHAF1B HMGA1 |
| | | | RBBP8 |
| 0.000020900 | 5 | Chromatin assembly | CHAF1B SOX9 HMGA1 IPO4 CENPV |
| 0.000043800 | 5 | DNA packaging | CHAF1B SOX9 HMGA1 IPO4 CENPV |
| 0.047824624 | 5 | Regulation of cell | RPS3 MYC GATA3 SOX9 NPAS2 |
| | | death | |
| 0.000031328 | 5 | Nucleosome | CHAF1B SOX9 IPO4 BRDT HMGA1 |
| | | organization | |
| 0.024677195 | 2 | Positive regulation of | MYC RPS3 |
| | | cysteine-type | |
| | | endopeptidase | |
| | | activity | |
| 0.017766778 | 2 | Nucleotide-excision | RBBP8 LIG1 |
| | | repair | |
| 0.044696654 | 2 | Double-strand break | RBBP8 HELB |
| | | repair | |
| 0.02021832 | 2 | Double-strand break | RBBP8 HELB |
| | | repair via | |
| | | homologous | |
| | | recombination | |

At time point 3, similarly the group of DEGs were not only involved in DNA repair, chromatin remodeling and organization but also showed their lasting effects on cell development and differentiation, DNA-templated transcription participating more in significant cell death and apoptosis processes (Table 4 and Table 5).

TABLE 4

| Enrichment | Genes | Functional | |
|---|---|---|---|
| FDR | in list | Category | Up-regulated genes |
| 9.09E−05 | 25 | Cell | JAK2 COL6A3 PITX2 PDGFB ITGB3 ABCB5 |
| | | differentiation | TLL1 PREX2 HDAC9 CAMK2A CRMP1 |
| | | | MECOM NOX4 CDC25B TWIST1 RAMP2 |
| | | | DYSF ACVRL1 ASAP1 CTHRC1 COL3A1 |
| | | | CBX2 EBF2 RUNX1T1 AICDA |
| 0.000326261 | 16 | Cell development | COL6A3 ITGB3 PREX2 CAMK2A CRMP1 |
| | | | JAK2 CDC25B TWIST1 RAMP2 DYSF ASAP1 |
| | | | CTHRC1 COL3A1 HDAC9 PDGFB PITX2 |
| 0.012275762 | 16 | Nucleic acid- | RUNX1T1 ZNF366 EBF2 HDAC9 MECOM |
| | | templated | PDGFB AICDA TWIST1 ACVRL1 PITX2 F2R |
| | | transcription | TMEM173 JAK2 CBX2 CAMK2A MAP3K12 |
| 0.012760221 | 16 | RNA | RUNX1T1 ZNF366 EBF2 HDAC9 MECOM |
| | | biosynthetic | PDGFB AICDA TWIST1 ACVRL1 PITX2 F2R |
| | | process | TMEM173 JAK2 CBX2 CAMK2A MAP3K12 |
| 0.014365858 | 16 | Regulation of | RUNX1T1 ZNF366 EBF2 HDAC9 MECOM |
| | | RNA metabolic | PDGFB AICDA TWIST1 ACVRL1 PITX2 F2R |
| | | process | TMEM173 JAK2 CBX2 CAMK2A MAP3K12 |
| 0.018259804 | 16 | Regulation of | ITGB3 JAK2 PTGFR F2R ABCB5 HDAC9 |
| | | biological quality | PDGFB RAMP2 HDC CAMK2A NOX4 DYSF |
| | | | EBF2 RAMP3 ACVRL1 COL3A1 |
| 0.021707241 | 16 | Cellular protein | JAK2 PDGFB MAP3K12 HDAC9 CAMK2A |
| | | modification | CDC25B ACVRL1 COL3A1 F2R PLPP1 |
| | | process | MECOM NOX4 TWIST1 DYSF RAMP3 ITGB3 |
| 0.001032276 | 14 | Cell proliferation | PDGFB F2R NOX4 JAK2 TWIST1 DYSF |
| | | | ACVRL1 CTHRC1 PTGFR ITGB3 MECOM |
| | | | PITX2 PLPP1 CDC25B |
| 0.035454549 | 13 | Immune system | JAK2 SAMHD1 AICDA TMEM173 PDGFB |
| | | process | DYSF RAB33A COL3A1 MECOM PITX2 |
| | | | HDAC9 CAMK2A ITGB3 |
| 0.017730779 | 11 | Cell death | RAMP2 F2R CAMK2A MECOM NOX4 JAK2 |
| | | | PTGFR TWIST1 MAP3K12 TMEM173 RAMP3 |
| 0.00226875 | 10 | Peptidyl-amino | JAK2 HDAC9 PDGFB CAMK2A MECOM |
| | | acid modification | NOX4 TWIST1 MAP3K12 RAMP3 ITGB3 |
| 0.007317162 | 10 | Secretion by cell | HDAC9 F2R CAMK2A JAK2 DYSF TWIST1 |
| | | | PDGFB PCDH7 TMEM173 ITGB3 |
| 0.018931741 | 10 | Apoptotic | RAMP2 F2R CAMK2A MECOM NOX4 JAK2 |
| | | process | PTGFR TWIST1 MAP3K12 TMEM173 |
| 0.026966123 | 10 | Programmed cell | RAMP2 F2R CAMK2A MECOM NOX4 JAK2 |
| | | death | PTGFR TWIST1 MAP3K12 TMEM173 |
| 0.00226875 | 9 | Tube | RAMP3 RAMP2 TWIST1 DYSF ACVRL1 |
| | | development | CTHRC1 COL3A1 HDAC9 ITGB3 |
| 0.01131492 | 9 | G protein- | PREX2 PTGFR RAMP3 RAMP2 F2R CAMK2A |
| | | coupled receptor | JAK2 GPR85 PLPP1 |
| | | signaling | |
| | | pathway | |
| 0.01199984 | 9 | Cell activation | PDGFB F2R JAK2 AICDA DYSF ITGB3 |
| | | | COL3A1 HDAC9 TMEM173 |
| 0.036968837 | 9 | Defense response | NOX4 SAMHD1 AICDA PTGFR TMEM173 |
| | | | JAK2 F2R HDAC9 CAMK2A |
| 0.032798532 | 8 | Regulation of | RAMP2 F2R CAMK2A NOX4 JAK2 PTGFR |
| | | apoptotic process | TWIST1 MAP3K12 |
| 0.001751147 | 7 | Wound healing | ITGB3 PDGFB COL3A1 F2R DYSF ACVRL1 |
| | | | JAK2 |
| 0.006433963 | 7 | Chromatin | JAK2 HDAC9 AICDA MECOM TWIST1 |
| | | organization | MAP3K12 CBX2 |
| 0.011833817 | 7 | MAPK cascade | PDGFB MAP3K12 F2R NOX4 JAK2 RAMP3 |
| | | | MECOM |
| 0.012191246 | 7 | Signal | PDGFB MAP3K12 F2R NOX4 JAK2 RAMP3 |
| | | transduction by | MECOM |
| | | protein | |
| | | phosphorylation | |
| 0.029649335 | 7 | Chromosome | JAK2 HDAC9 AICDA MECOM TWIST1 |
| | | organization | MAP3K12 CBX2 |
| 0.003544678 | 6 | Covalent | JAK2 HDAC9 AICDA MECOM TWIST1 |
| | | chromatin | MAP3K12 |
| | | modification | |
| 0.005693484 | 6 | Small GTPase | RAB33A F2R PREX2 PLEKHG1 COL3A1 JAK2 |
| | | mediated signal | |
| | | transduction | |
| 0.018013979 | 6 | Inflammatory | PTGFR JAK2 F2R TMEM173 HDAC9 NOX4 |
| | | response | |
| 0.039647739 | 6 | DNA metabolic | AICDA PDGFB SAMHD1 NOX4 TWIST1 |
| | | process | ACVRL1 |
| 0.000584671 | 5 | Phosphatidylinos | PDGFB PREX2 F2R TWIST1 JAK2 |
| | | itol 3-kinase | |
| | | signaling | |
| 0.003674696 | 5 | ERK1 and ERK2 | PDGFB NOX4 F2R RAMP3 MAP3K12 |
| | | cascade | |
| 0.009967432 | 5 | Histone | JAK2 HDAC9 MECOM TWIST1 MAP3K12 |
| | | modification | |
| 0.00493481 | 4 | Rho protein | F2R PREX2 PLEKHG1 COL3A1 |
| | | signal | |
| | | transduction | |
| 0.018852283 | 4 | Peptidyl-tyrosine | JAK2 PDGFB NOX4 ITGB3 |
| | | modification | |
| 0.034790962 | 4 | Regulation of | TMEM173 JAK2 TWIST1 CAMK2A |
| | | DNA-binding | |
| | | transcription | |
| | | factor activity | |
| 0.010229704 | 3 | Adenylate | PTGFR RAMP3 RAMP2 |
| | | cyclase- | |
| | | activating G | |
| | | protein-coupled | |
| | | receptor | |
| | | signaling | |
| | | pathway | |
| 0.01743615 | 3 | CAMP-mediated | PTGFR RAMP3 RAMP2 |
| | | signaling | |
| 0.018931741 | 3 | DNA | PDGFB NOX4 ACVRL1 |
| | | biosynthetic | |
| | | process | |
| 0.019121306 | 3 | Steroid hormone | ZNF366 JAK2 PLPP1 |
| | | mediated | |
| | | signaling | |
| | | pathway | |
| 0.022907215 | 3 | Cyclic- | PTGFR RAMP3 RAMP2 |
| | | nucleotide- | |
| | | mediated | |
| | | signaling | |
| 0.025253485 | 3 | Adenylate | PTGFR RAMP3 RAMP2 |
| | | cyclase- | |
| | | modulating G | |
| | | protein-coupled | |
| | | receptor | |
| | | signaling | |
| | | pathway | |
| 0.0262597 | 3 | Fat cell | DYSF EBF2 RUNX1T1 |
| | | differentiation | |
| 0.027608538 | 3 | Cell-matrix | ITGB3 COL3A1 ACVRL1 |
| | | adhesion | |
| 0.032841118 | 3 | Protein kinase B | NOX4 RAMP3 PDGFB |
| | | signaling | |
| 0.032934614 | 3 | G protein- | PTGFR RAMP3 RAMP2 |
| | | coupled receptor | |
| | | signaling | |
| | | pathway, coupled | |
| | | to cyclic | |
| | | nucleotide | |
| | | second | |
| | | messenger | |
| 0.032934614 | 3 | Defense response | SAMHD1 AICDA TMEM173 |
| | | to virus | |
| 0.035285992 | 3 | Mesenchyme | TWIST1 ACVRL1 PITX2 |
| | | development | |
| 0.039485793 | 3 | DNA replication | SAMHD1 AICDA ACVRL1 |
| 0.007317162 | 2 | Protein kinase C- | F2R PLPP1 |
| | | activating G | |
| | | protein-coupled | |
| | | receptor | |
| | | signaling | |
| | | pathway | |
| 0.042471441 | 2 | Integrin- | ITGB3 COL3A1 |
| | | mediated | |
| | | signaling | |
| | | pathway | |
| 0.043485782 | 2 | Regulation of | AICDA ACVRL1 |
| | | DNA replication | |
| 0.043814313 | 2 | Epithelial cell | RAMP2 JAK2 |
| | | apoptotic process | |
| 4.93E−07 | 13 | Nucleic acid- | TAL1 HDAC9 MECOM PDGFB AICDA |
| | | templated | TWIST1 ACVRL1 GATA2 TMEM173 JAK2 |
| | | transcription | CBX2 CAMK2A MAP3K12 |
| 5.06E−07 | 13 | RNA | TAL1 HDAC9 MECOM PDGFB AICDA |
| | | biosynthetic | TWIST1 ACVRL1 GATA2 TMEM173 JAK2 |
| | | process | CBX2 CAMK2A MAP3K12 |
| 4.78E−07 | 13 | Transcription, | TAL1 HDAC9 MECOM PDGFB AICDA |
| | | DNA-templated | TWIST1 ACVRL1 GATA2 TMEM173 JAK2 |
| | | | CBX2 CAMK2A MAP3K12 |
| 1.10E−05 | 12 | Cell | JAK2 PDGFB TAL1 GATA2 HDAC9 CAMK2A |
| | | differentiation | MECOM NOX4 TWIST1 ACVRL1 CBX2 |
| | | | AICDA |
| 6.68E−05 | 11 | Cellular protein | JAK2 PDGFB MAP3K12 HDAC9 CAMK2A |
| | | modification | ACVRL1 MECOM NOX4 TWIST1 TAL1 |
| | | process | GATA2 |
| 9.07E−08 | 10 | Peptidyl-amino | JAK2 HDAC9 PDGFB CAMK2A MECOM |
| | | acid modification | NOX4 TWIST1 MAP3K12 TAL1 GATA2 |
| 7.63E−05 | 10 | Immune system | JAK2 SAMHD1 AICDA TMEM173 PDGFB |
| | | process | TAL1 GATA2 MECOM HDAC9 CAMK2A |
| 6.72E−07 | 9 | Chromosome | JAK2 HDAC9 AICDA MECOM TWIST1 |
| | | organization | MAP3K12 CBX2 GATA2 TAL1 |
| 7.71E−08 | 9 | Chromatin | JAK2 HDAC9 AICDA MECOM TWIST1 |
| | | organization | MAP3K12 CBX2 GATA2 TAL1 |
| 0.000112872 | 8 | Apoptotic | CAMK2A MECOM NOX4 JAK2 TWIST1 |
| | | process | MAP3K12 TMEM173 GATA2 |
| 0.000130591 | 8 | Cell proliferation | PDGFB GATA2 NOX4 JAK2 TWIST1 ACVRL1 |
| | | | TAL1 MECOM |
| 0.000242121 | 8 | Cell death | CAMK2A MECOM NOX4 JAK2 TWIST1 |
| | | | MAP3K12 TMEM173 GATA2 |
| 4.93E−07 | 7 | Covalent | JAK2 HDAC9 AICDA MECOM TWIST1 |
| | | chromatin | MAP3K12 GATA2 |
| | | modification | |
| 0.000778169 | 7 | Tissue | PDGFB NOX4 TWIST1 ACVRL1 TAL1 |
| | | development | HDAC9 JAK2 |
| 0.000241941 | 7 | Secretion by cell | HDAC9 CAMK2A JAK2 GATA2 TWIST1 |
| | | | PDGFB TMEM173 |
| 0.000881571 | 7 | Cell development | GATA2 CAMK2A JAK2 TWIST1 TAL1 |
| | | | HDAC9 PDGFB |
| 0.000204001 | 6 | DNA metabolic | AICDA PDGFB SAMHD1 NOX4 TWIST1 |
| | | process | ACVRL1 |
| 0.0012293 | 6 | Regulation of | CAMK2A NOX4 JAK2 TWIST1 MAP3K12 |
| | | apoptotic process | GATA2 |
| 0.001043083 | 6 | Cell activation | PDGFB JAK2 AICDA GATA2 HDAC9 |
| | | | TMEM173 |
| 6.28E−06 | 6 | Histone | JAK2 HDAC9 MECOM TWIST1 MAP3K12 |
| | | modification | GATA2 |
| 0.001112152 | 5 | Signal | PDGFB MAP3K12 NOX4 JAK2 MECOM |
| | | transduction by | |
| | | protein | |
| | | phosphorylation | |
| 0.001071587 | 5 | MAPK cascade | PDGFB MAP3K12 NOX4 JAK2 MECOM |
| 0.000380828 | 4 | Peptidyl-tyrosine | JAK2 PDGFB NOX4 TAL1 |
| | | modification | |
| 0.004184216 | 4 | Inflammatory | JAK2 TMEM173 HDAC9 NOX4 |
| | | response | |
| 0.001326118 | 4 | Wound healing | PDGFB ACVRL1 JAK2 GATA2 |
| 0.000530278 | 3 | Phosphatidylinos | PDGFB TWIST1 JAK2 |
| | | itol 3-kinase | |
| | | signaling | |
| 0.002940517 | 3 | ERK1 and ERK2 | PDGFB NOX4 MAP3K12 |
| | | cascade | |
| 0.002102052 | 3 | DNA replication | SAMHD1 AICDA ACVRL1 |
| 0.00089215 | 3 | DNA | PDGFB NOX4 ACVRL1 |
| | | biosynthetic | |
| | | process | |
| 0.005461523 | 2 | Regulation of | AICDA ACVRL1 |
| | | DNA replication | |
| 0.017911294 | 2 | Protein kinase B | NOX4 PDGFB |
| | | signaling | |
| 0.019095449 | 2 | Mesenchyme | TWIST1 ACVRL1 |
| | | development | |
| 0.005520485 | 2 | Epithelial cell | JAK2 GATA2 |
| | | apoptotic process | |

TABLE 5

| Enrichment | Genes | Functional | |
|---|---|---|---|
| FDR | in list | Category | Down-regulated genes |
| 7.28E−06 | 6 | Chromatin | EYA2 PADI2 CHAF1B HMGA1 L3MBTL4 |
| | | organization | CENPV |
| 2.94E−05 | 6 | Chromosome | EYA2 PADI2 CHAF1B HMGA1 L3MBTL4 |
| | | organization | CENPV |
| 0.018425809 | 5 | Nucleic acid- | HMGA1 NPAS2 L3MBTL4 CDCA7L PADI2 |
| | | templated | |
| | | transcription | |
| 0.018425809 | 5 | Transcription, | HMGA1 NPAS2 L3MBTL4 CDCA7L PADI2 |
| | | DNA-templated | |
| 0.018425809 | 5 | RNA | HMGA1 NPAS2 L3MBTL4 CDCA7L PADI2 |
| | | biosynthetic | |
| | | process | |
| 0.026855905 | 5 | Regulation of | HMGA1 NPAS2 L3MBTL4 CDCA7L PADI2 |
| | | biosynthetic | |
| | | process | |
| 0.026855905 | 5 | Heterocycle | HMGA1 NPAS2 L3MBTL4 CDCA7L PADI2 |
| | | biosynthetic | |
| | | process | |
| 0.026855905 | 5 | Nucleobase- | HMGA1 NPAS2 L3MBTL4 CDCA7L PADI2 |
| | | containing | |
| | | compound | |
| | | biosynthetic | |
| | | process | |
| 2.23E−05 | 4 | Chromatin | CHAF1B PADI2 HMGA1 CENPV |
| | | assembly or | |
| | | disassembly | |
| 0.000371098 | 4 | DNA repair | EYA2 HMGA1 CHAF1B NPAS2 |
| 0.00144086 | 4 | Cellular response | EYA2 HMGA1 CHAF1B NPAS2 |
| | | to DNA damage | |
| | | stimulus | |
| 0.001947444 | 4 | DNA metabolic | EYA2 HMGA1 CHAF1B NPAS2 |
| | | process | |
| 0.000371098 | 3 | Chromatin | CHAF1B HMGA1 CENPV |
| | | assembly | |
| 0.000371983 | 3 | Chromatin | HMGA1 PADI2 CENPV |
| | | remodeling | |
| 0.000509154 | 3 | DNA packaging | CHAF1B HMGA1 CENPV |
| 0.001263719 | 3 | Protein-DNA | CHAF1B CENPV HMGA1 |
| | | complex subunit | |
| | | organization | |
| 0.001332106 | 3 | DNA | CHAF1B HMGA1 CENPV |
| | | conformation | |
| | | change | |
| 0.049569423 | 3 | Protein- | CHAF1B CENPV HMGA1 |
| | | containing | |
| | | complex | |
| | | assembly | |
| 0.000154705 | 2 | Heterochromatin | HMGA1 CENPV |
| | | assembly | |
| 0.000371098 | 2 | Chromatin | PADI2 HMGA1 |
| | | disassembly | |
| 0.000371098 | 2 | Heterochromatin | HMGA1 CENPV |
| | | organization | |
| 0.009231385 | 2 | Nucleosome | CHAF1B HMGA1 |
| | | organization | |
| 0.013840524 | 2 | Protein-DNA | CHAF1B CENPV |
| | | complex | |
| | | assembly | |
| 0.015582247 | 2 | DNA replication | CHAF1B HMGA1 |
| 0.026855905 | 2 | Covalent | EYA2 PADI2 |
| | | chromatin | |
| | | modification | |
| 0.026855905 | 2 | Histone | EYA2 PADI2 |
| | | modification | |
| 0.031503726 | 2 | Cellular | PADI2 HMGA1 |
| | | component | |
| | | disassembly | |
| 0.035376826 | 2 | Cell division | CDCA7L CENPV |
| 7.28E−06 | 6 | Chromatin | EYA2 PADI2 CHAF1B HMGA1 L3MBTL4 |
| | | organization | CENPV |
| 2.94E−05 | 6 | Chromosome | EYA2 PADI2 CHAF1B HMGA1 L3MBTL4 |
| | | organization | CENPV |
| 2.23E−05 | 4 | Chromatin | CHAF1B PADI2 HMGA1 CENPV |
| | | assembly or | |
| | | disassembly | |

Gene expression change induced by rhCG has an impact on activating upstream regulators TGFβ, TP53, BRCA1, and TP53 and on suppressing canonical wntβ-catenin signaling and MYC in breasts of BRCA1/2 mutation carriers unexposed to contraceptives. To identify the canonical pathways and upstream regulators of DEGs affected by rhCG treatment in breast tissues of BRCA1/2 mutation carrier women, DEGs induced by rhCG were used for the analysis using IPA (Qiagen, USA). Significant pathway or regulator enrichment was determined to be activated with positive z-score and inhibited with negative z-score and the expression FDRp<0.05 (q value) for gene expression in network, in which z-score is the statistical measure of correlation between relationship direction and gene expression. At both time point 2 and time point 3, Wnt/β-catenin and PPAR signaling were inhibited while p38 MAPK signaling was activated (data not shown).

For the upstream regulators, based on the analysis of target genes, TGFβ1, TGFβ2, TGFβR1, and TGFβR2 were persistently predicted to be activated whereas MYC was inhibited at both time point 2 and time point 3 (data not shown). The activity of BRCA1 and TP53 were predicted to be activated at both time point 2 and time point 3, with more significant change at time point 3 (Z-score for BRCA1 1.039 at time point 2 and 2.049 at time point 3; Z-score for TP53 1.99 at time point 2 and 2.711 at time point 3).

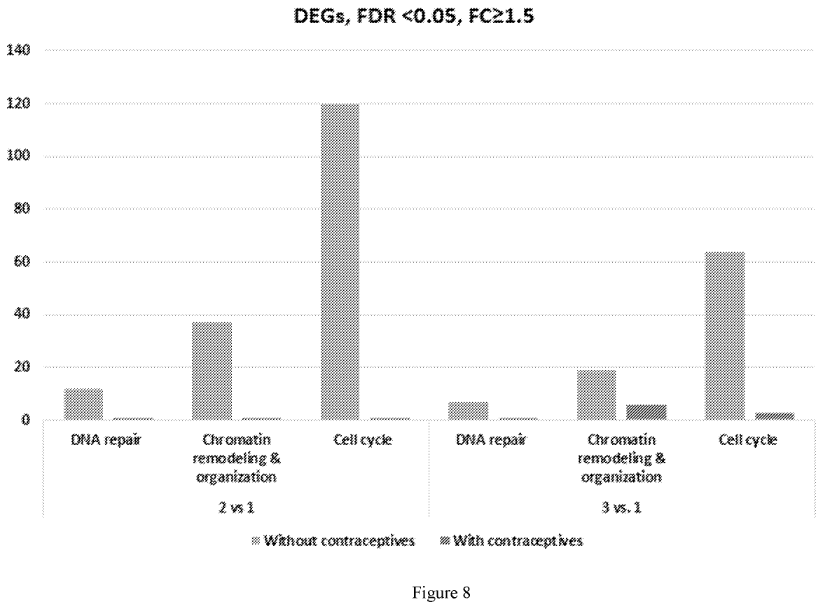

Effects of rhCG on the changes of gene expression is markedly reduced and delayed by the exposure to contraceptives, with the vanishment of all DEGs related to BRCA1 activation and DNA repair. In the present study, a clear difference between the two groups was observed in the response to the rhCG at both T2 and T3. While there are 1907 DEGs (1032 up, 875 down) at T2 and 1065 DEGs (897 up, 168 down) at T3 for the women group (n=11) without exposure to contraceptives, there are almost no response at T2 and a small number of DEGs, 260 (214 up, 46 down) at T3 for the group of women with hormonal birth control use (n=14) (FIG. 3). An expression heatmap was generated which showed the reduction in gene expression changes and the delayed response to rhCG in breasts of these women exposed to hormonal birth control until 6 months post rhCG therapy. Similarly, the less significance of the differentially expressed genes and the postponed effect of rhCG to 6 months after treatment on the low-responder's breasts are shown in the volcano plots (data not shown). For this group, there was also no DEGs related to DNA repair at both T2 and T3, and very low levels of DEGs associated with chromatin remodeling and organization (6 DEGs) as well as cell cycle (3 DEGs) (FIG. 8). No DEGs were found as target genes for prediction of upstream BRCA1 activation even at 6 months after rhCG therapy termination. These all indicate that hormonal contraceptives cause the abolition of pregnancy mimicking effect of rhCG on breast of women carrying BRCA1/2 mutation. This is the first report that the rhCG effect is interacted with hormonal contraceptives in BRCA1/2 mutation carriers, which not only causes the considerable reduction of number of differently expressed genes, but also eliminates critical effects of rhCG in DNA damage repair, chromatin remodeling and organization as well as BRCA1 activation.

The present study is the first report showing the pregnancy mimicking effect of rhCG on the genomic signature induced by rhCG in women carrying BRCA1/2 mutation and that of parous women in suppressing Wnt/β-signaling and chromatin remodeling. Moreover, rhCG also its effect on activating BRCA1 and TP53 in breast of BRCA1/2 mutation carriers, which are known as “protector of genome stability” against the breast cancer development.

Taken together, it is concluded that rhCG has a great remarkable effect on the transcriptomic profile of breast tissues from women carrying deleterious BRCA1/2 mutation towards the protective signaling against breast cancer development. The major relevant and significant effects of rhCG are activating the TGFβ signaling, cell proliferation-differentiation, DNA repair, chromatin remodeling and organization as well as suppressing Wnt/β-catenin signaling. Gene expression changes induced by rhCG additionally triggers the activation of TGFβ, TGFβR, BRCA1, and TP53 and the inhibition of MYC. These findings suggest that rhCG plays an important role in breast cancer prevention and this effect is long lasting. Moreover, when using rhCG as a preventive therapy from breast cancer, the role of contraceptives should be considered since it proves that contraceptives use can interact with rhCG and cause a reduced or delayed response of gene profile alteration to rhCG therapy.

### Example 4: Immunohistochemical Analysis

Immunohistochemical analysis demonstrated rhCG induced upregulation of BRCA1 and chromatin remodeling in breast tissues of BRCA1/2 carriers. rhCG treatment also up-regulated BRCA1 and FOXO3A expression in breast epithelial cells of BRCA1/2 carrier women.

The analysis of RNA-sequencing of breast tissue from rhCG treated women BRCA1/2 carriers showed that WNT/β-catenin signaling was inhibited while TGFβ signaling and BRCA1 were activated. To determine whether the inhibition of WNT/beta-catenin signaling and activation of TGF beta signaling might result in up-regulation of BRCA1 expression, immunohistochemical analysis was performed using breast tissues from BRCA1/2 mutation carriers.

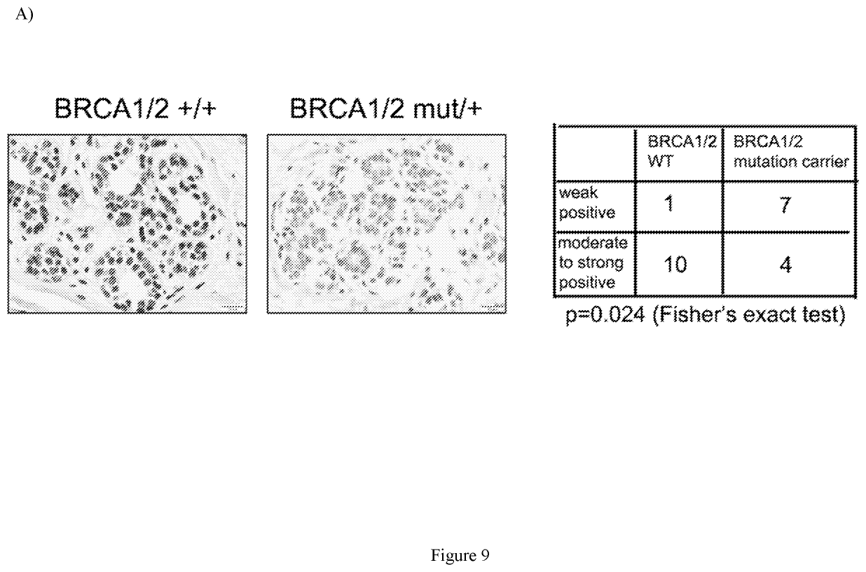

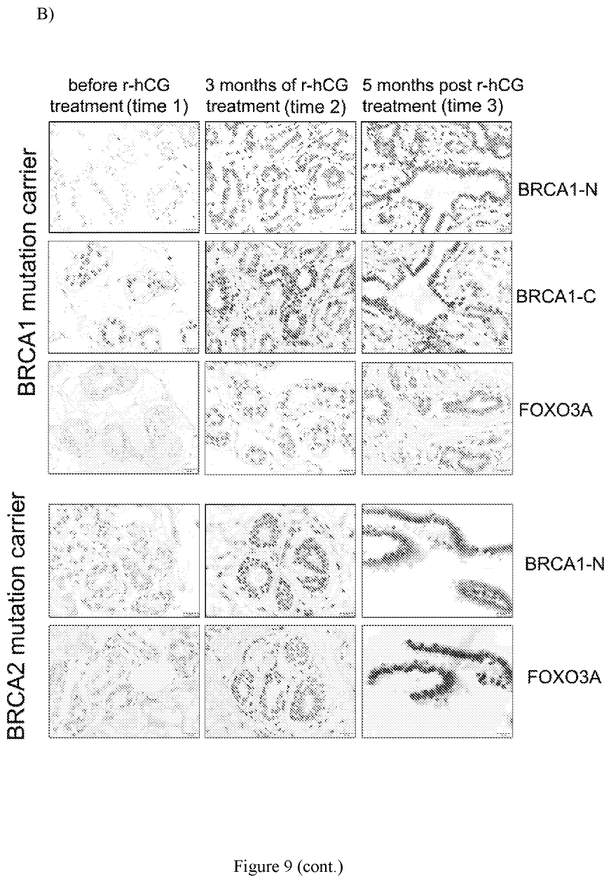

The expression of BRCA1 in the breast tissues of BRCA1/2 wild type women and BRCA1/2 mutation carriers prior to rhCG treatment was compared. BRCA1 protein was significantly higher in the breast tissues of BRCA1/2 wild type women (FIG. 9A), and BRCA1 mRNA was decreased in peripheral blood leukocytes of cancer free BRCA1 mutation carriers, supporting the concept of BRCA1 haploinsufficiency for BRCA1 mutation cells. FIG. 9B shows representative IHC images of BRCA1 or BRCA2 mutation carriers without contraceptives use. To determine whether BRCA1 expression was affected by rhCG treatment in the breast tissues of BRCA1/2 carrier women, two different monoclonal BRCA1 antibodies from Abcam were used to evaluate the expression of BRCA1 in these samples since the mutation of BRCA1 in these 33 patients cover different parts of the gene. One antibody was anti-BRCA1 (clone MS 110) that recognizes an epitope within N-terminal BRCA1 amino acids 89-222 (named as BRCA1-N here), and the other was anti-BRCA1 (clone EPR19433) that recognizes C-terminal BRCA1 amino acids 1700-1800 (named as BRCA1-C). Theoretically, the BRCA1-N antibody will detect total BRCA1 protein, including wild type and mutant protein. Since some mutations result in frame shift and premature termination of stop codon of BRCA1 gene, for these kinds of patients, BRCA-C antibody will detect wild type BRCA1 only. The actual expression of mutant BRCA1 in these cells is unknown.

In both BRCA1 mutation and BRCA2 mutation carriers, it was observed that the change of BRCA1 expression was different from subject to subject. It was found that the effect of rhCG treatment on BRCA1 expression was related to the use of contraceptives. In these 33 subjects, some subject did not use contraceptives, some used oral contraceptives, and stopped prior to the rhCG treatment, and some had the intrauterine device or oral contraceptives throughout the study. For the subjects without contraceptives (including women who did not use contraceptives and who stopped oral contraceptives more than 30 days prior to the rhCG treatment), a trend of increase of BRCA1 protein at time point 3 (7 out of 9 patients, p=0.0956, 2-sided test of binomical proportion test) was observed when using BRCA1-N antibody. There was a significant increase of BRCA1 protein at time point 2 when BRCA1-C antibody was used (8 out of 9 patients, p=0.0196, 2-sided test of binomical proportion test). Some subjects who used contraceptives during the study had an increase of BRCA1 at time point 3 (5 out of 7 patients, p=0.23). The use of contraceptives during the study delayed the response of BRCA1 evaluated by BRCA-C antibody. The subjects who stopped oral contraceptives less than 30 days prior to the study did not have a response of BRCA1 increase (Table 6).

TABLE 6

| Time 1 to 2 | | | | | | Time 1 to 3 | | | | | |
|---|---|---|---|---|---|---|---|---|---|---|---|
| | up-regulation of BRCA1 | | | | Total | | up-regulation of BRCA1 | | | | Total |
| BRCA1-N Ab | Yes | | No | | subjects | BRCA1-N Ab | Yes | | No | | subjects |
| No contraceptives | 5 | (55.6%) | 4 | (44.4%) | 9 | No contraceptives | 7 | (77.8%) | 2 | (22.2%) | 9 |
| Stop oral | 4 | (40.0%) | 6 | (60.0%) | 10 | Stop oral | 5 | (50.0%) | 5 | (50.0%) | 10 |
| contraceptives | | | | | | contraceptives | | | | | |
| less than 30 | | | | | | less than 30 | | | | | |
| days prior to | | | | | | days prior to | | | | | |
| study | | | | | | study | | | | | |
| Contraceptives | 3 | (37.5%) | 5 | (62.5%) | 8 | Contraceptives | 3 | (37.5%) | 5 | (62.5%) | 8 |
| use during the | | | | | | use during the | | | | | |
| study | | | | | | study | | | | | |
| Total subjects | 13 | (48.1%) | 14 | (51.9%) | 27 | Total subjects | 16 | (59.3%) | 11 | (40.7%) | 27 |
| Time 1 to 2 | | | | | | Time 1 to 3 | | | | | |
| | up-regulation of BRCA1 | | | | Total | | up-regulation of BRCA1 | | | | Total |
| BRCA1-C Ab | Yes | | No | | subjects | BRCA1-C Ab | Yes | | No | | subjects |
| No contraceptives | 8 | (88.9%) | 1 | (11.1%) | 9 | No contraceptives | 5 | (55.6%) | 4 | (44.4%) | 9 |
| Stop oral | 3 | (37.5%) | 5 | (62.5%) | 8 | Stop oral | 2 | (25.0%) | 6 | (75.0%) | 8 |
| contraceptives | | | | | | contraceptives | | | | | |
| less than 30 | | | | | | less than 30 | | | | | |
| days prior to | | | | | | days prior to | | | | | |
| study | | | | | | study | | | | | |
| Contraceptives | 3 | (42.9%) | 4 | (57.1%) | 7 | Contraceptives | 5 | (71.4%) | 2 | (28.6%) | 7 |
| use during the | | | | | | use during the | | | | | |
| study | | | | | | study | | | | | |
| Total subjects | 13 | (54.2%) | 11 | (45.8%) | 24 | Total subjects | 13 | (54.2%) | 11 | (45.8%) | 24 |

| Time 1 to 2 | | | | Time 1 to 3 | | | |
|---|---|---|---|---|---|---|---|
| | up-regulation of FOXO3A | | Total | | up-regulation of FOXO3A | | Total |
| FOXO3A | Yes | No | subjects | FOXO3A | Yes | No | subjects |
| No contraceptives | 7 (77.8%) | 2 (22.2%) | 9 | No contraceptives | 6 (66.7%) | 3 (33.3%) | 9 |
| Stop oral | 7 (70.0%) | 3 (30.0%) | 10 | Stop oral | 6 (60.0%) | 4 (40.0%) | 10 |
| contraceptives | | | | contraceptives | | | |
| less than 30 | | | | less than 30 | | | |
| days prior to | | | | days prior to | | | |
| study | | | | study | | | |
| Contraceptives | 2 (25.0%) | 6 (75.0%) | 8 | Contraceptives | 5 (62.5%) | 3 (37.5%) | 8 |
| use during the | | | | use during the | | | |
| study | | | | study | | | |
| Total subjects | 16 (59.3%) | 11 (40.7%) | 27 | Total subjects | 17 (63.0%) | 11 (45.8%) | 27 |

The expression of one of the BRCA1 target genes, FOXO3A, was also evaluated. The effect of rhCG on FOXO3A expression was similar to that on BRCA1 expression evaluated by BRCA1-C antibody, which suggests the level of full length BRCA1 protein is more likely to be associated with the expression of FOXO3A. There was a trend of increase of FOXO3A at time point 2 for subjects without contraceptives use (7 out of 9 subjects, p=0.0956, 2-sided test of binomical proportion test). At time points 3, 6, and 9, subjects had an increase of FOXO3A (p=0.3173, 2-sided test of binomical proportion test). Some subjects who stopped oral contraceptives use less than 30 days prior to rhCG treatment also had an increase of FOXO3A at time point 2 (7 out of 10 subjects, p=0.18), which is different from the response of BRCA1, suggesting FOXO3A is also regulated by other genes and pathways. Taken together, these data indicate that BRCA1 protein expression is reduced in the breast epithelial cells of BRCA1/2 mutation carriers, and rhCG treatment can induce BRCA1 and FOXO3A expression in these cells, demonstrating a possible role of rhCG in preventing breast carcinogenesis through recovery of BRCA1 function.

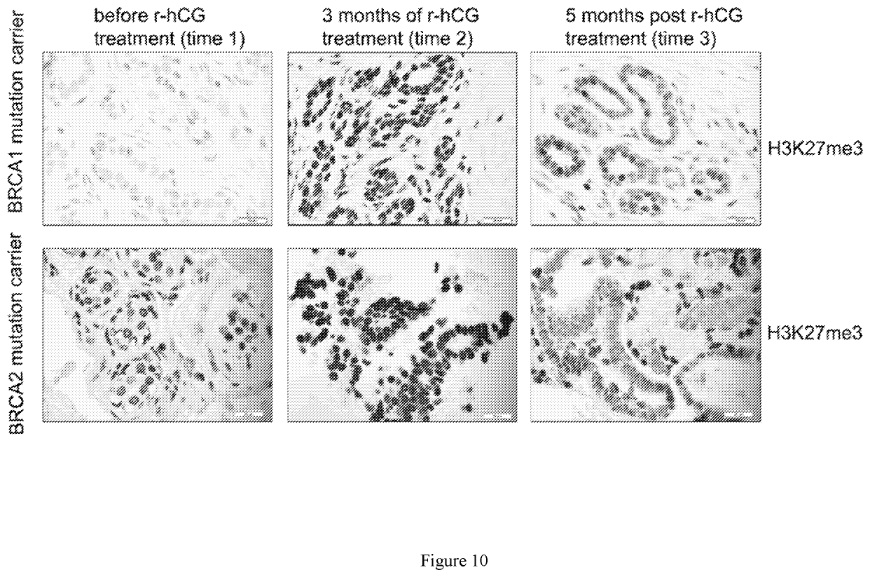

To explore the effect of rhCG on H3K27me3 in breast epithelial cells of BRCA1/2 carriers, IHC was performed on breast biopsy samples. Similar to the change of BRCA1, the use of contraceptives also affected the change of H3K27me3. Subjects without contraceptives tended to have an increase of H3K27me3 at time point 2 (6 out of 9 patients). In addition, subjects who had used contraceptives during the study showed a significant increase of H3K27me3 at time point 2 (7 out of 8 subjects, p=0.035). Interestingly, subjects who used oral contraceptives and stopped less than 30 days prior to rhCG treatment had almost an opposite response, only 20% of these women had H3K27me3 increase at time point 2, and 0% had H3K27me3 increase at time point 3 (FIG. 10 and Table 7). In summary, these observations indicate that rhCG induces chromatin remodeling in breast epithelial cells which could contribute to protection against breast cancer.

TABLE 7

| | up of H3K27me3 at time 2 | | | | Total |
|---|---|---|---|---|---|
| H3K27me3 | Yes | | No | | subjects |
| No contraceptives | 6 | (66.7%) | 3 | (33.3%) | 9 |
| Stop oral contraceptives less | 2 | (20%) | 8 | (80%) | 10 |
| than 30 days prior to study | | | | | |
| Contraceptives use during the | 7 | (87.5%) | 1 | (12.5%) | 8 |
| study | | | | | |
| Total subjects | 15 | (55.6%) | 12 | (44.4) | 27 |
| | up of H3K27me3 at time 3 | | | | Total |
| H3K27me3 | Yes | | No | | subjects |
| No contraceptives | 5 | (55.6%) | 4 | (44.4%) | 9 |
| Stop oral contraceptives less | 0 | (0.0%) | 10 | (100%) | 10 |
| than 30 days prior to study | | | | | |
| Contraceptives use during the | 4 | (50%) | 4 | (50%) | 8 |
| study | | | | | |
| Total subjects | 9 | (33.3%) | 18 | (66.6%) | 27 |

### Example 5: rhCG Induces Up-Regulation of Tumor Suppressors, Increases DNA Repair, and Induces Chromatin Remodeling in Breast Epithelial Cells In Vitro

### Recombinant hCG Up-Regulates BRCA1, BARD1, and FOXO3A Expression in Breast Epithelial Cells:

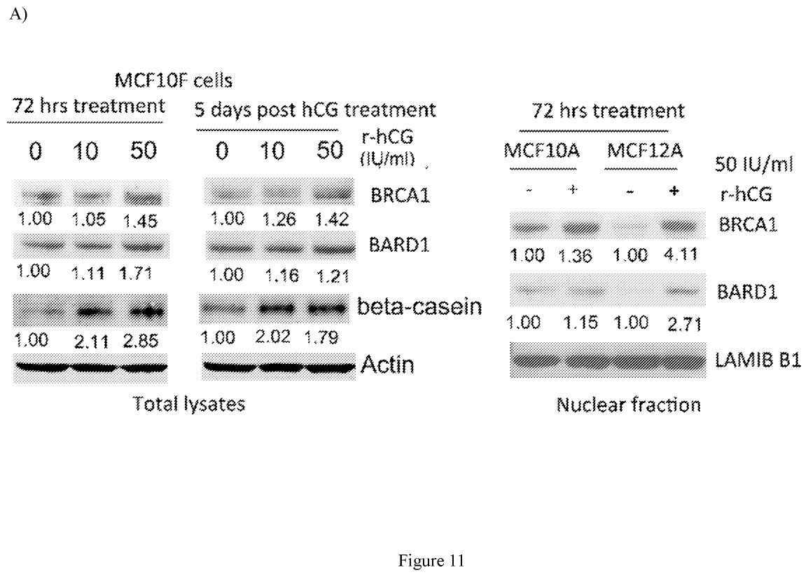

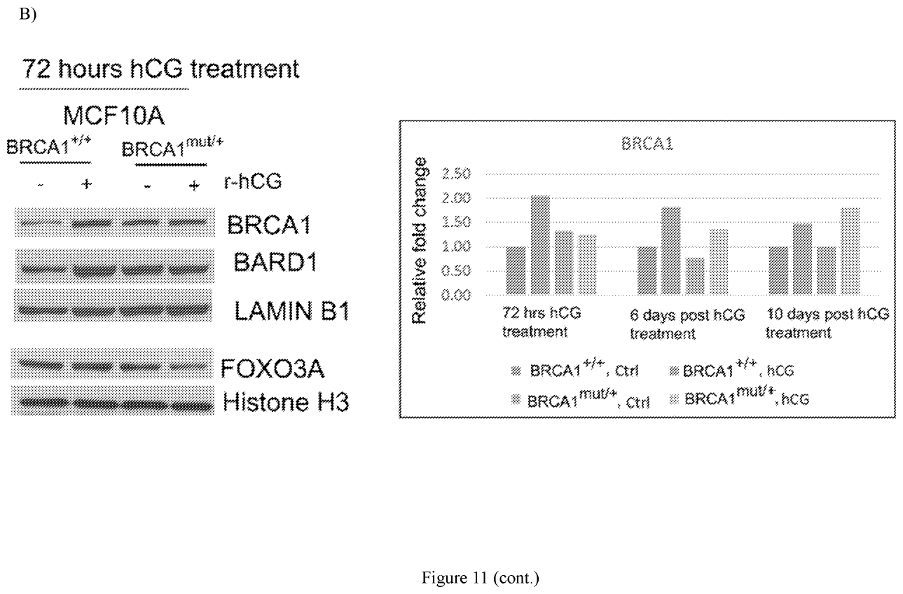

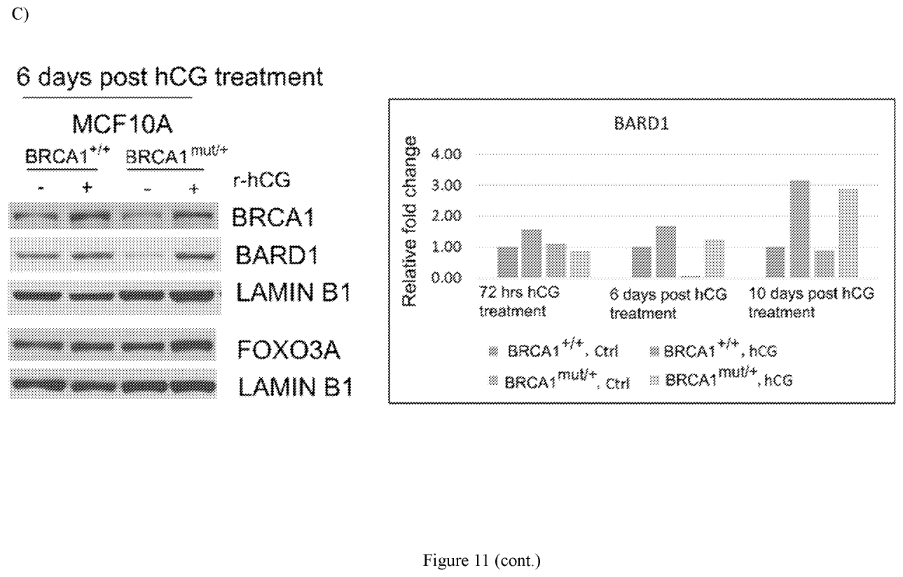

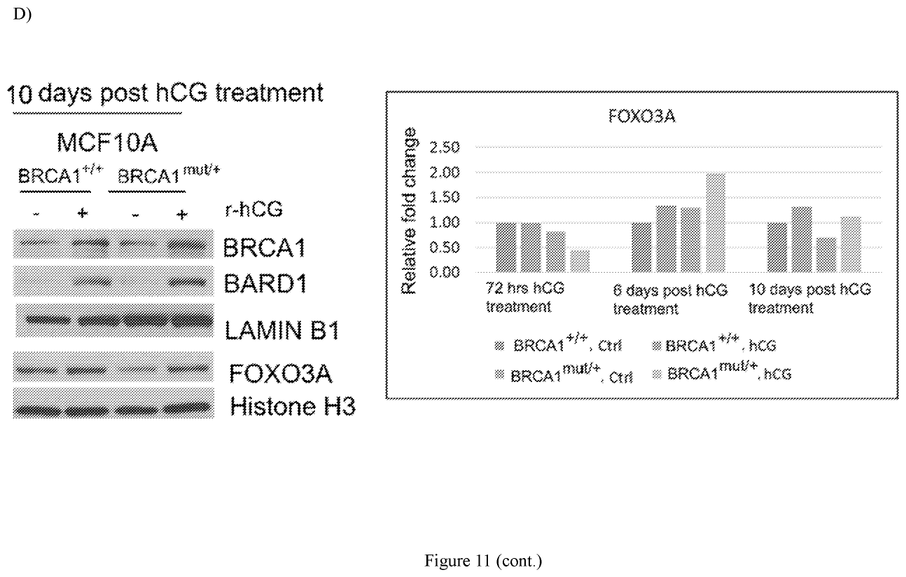

To determine whether rhCG could directly induce BRCA1 expression in breast epithelial cells, the breast epithelial cell line MCF10F was treated with 10 and 50 IU/ml of rhCG, and evaluated protein expression by Western blotting (WB). FIG. 11A shows that 50 IU/ml of rhCG treatment induced up-regulation of BRCA1 and BARD1 in MCF10F cells at the end of treatment and persisted 5-days post treatment stopped. BARD1 is a major partner of BRCA1, and it has nearly identical phenotype in knock-out mice. Consistently, it was also observed that rhCG induced beta-casein expression in MCF10F cells.

The up-regulation of BRCA1 and BARD1 was also demonstrated using other two breast epithelial cell lines: MCF10A and MCF12A (FIG. 11A). Both MCF10F and MCF10A cell lines were developed from the same parous woman, whereas the MCF12A cell line was derived from a nulliparous woman. The increase of both BRCA1 and BARD1 was greater in nulliparous cell line MCF12A than in parous cell line MCF10A because of a very low base level of the two proteins in MCF12A, this observation is consistent to the finding that the BRCA1 level is lower in the breast of nulliparous women compared to that of early parous women.

It was next determined whether rhCG could induce BRCA1 expression in BRCA1 mutant carrier breast epithelial cells. For this purpose, an MCF10A cell line with heterozygous knock-in of a 2-bp deletion in BRCA1 (185AG^{del/+}) resulting in a premature termination codon at position 39, hereafter termed BRCA1^{mut/+} MCF10A cell line, was used and the parental MCF10A cell line with wild type BRCA1 was used as a control (referred as BRCA1^{+/+}). BRCA1 and BARD1 (FIG. 11B and FIG. 11C) were evaluated at three different time points: at the end of 72 hours rhCG treatment, 6 days and 10 days post rhCG treatment. BRCA1 was significantly upregulated at all three time points in BRCA1^{+/+} MCF10A cells. For BRCA1^{mut/+} MCF10A cells, there was no change at the end of 72 hours rhCG treatment, whereas both 6 days and 10 days post rhCG treatment showed a significant increase of BRCA1. Since the BRCA1 185AG del causes a premature stop codon, and the BRCA1 antibody used for Western blotting is an antibody which recognizes amino acids 1842-1862 at the C-terminus of BRCA1 (the full length BRCA1 protein), it suggests that rhCG treatment could induce wild type BRCA1 expression in BRCA1^{mut/+} MCF10A cells. The effect of rhCG on BARD1 expression was very similar to that of BRCA1, exhibiting an upregulation of BARD1 at all three time points in BRCA^{+/+} cells, whereas in BRCA1^{mut/+} MCF10A cells the upregulation was only seen at 6 days and 10 days post rhCG treatment. BRCA1 is known to positively regulate FOXO3A gene expression in breast cancer cells. FOXO3A is a member of FOXO transcription factors which acts as a tumor suppressor gene, inhibits cell growth, controls DNA damage response, and associates with longevity. Consistent with the upregulation of BRCA1, the expression of FOXO3A was found to be increased in both BRCA1^{+/+} and BRCA1^{mut/+} MCF10A cells at 6 days and 10 days post rhCG treatment (FIG. 11D). Collectively, these date provide the evidence that rhCG induces the expression of BRCA1 and genes related to the BRCA1 function.

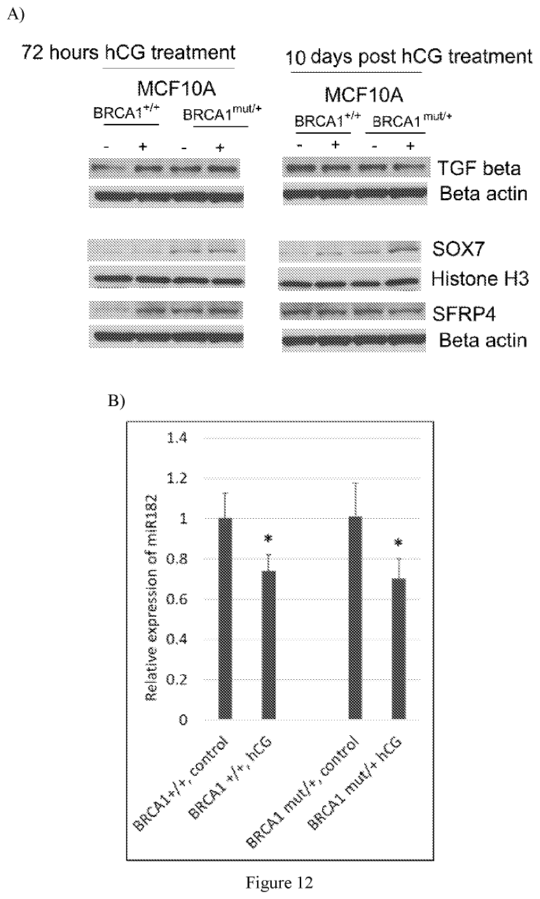

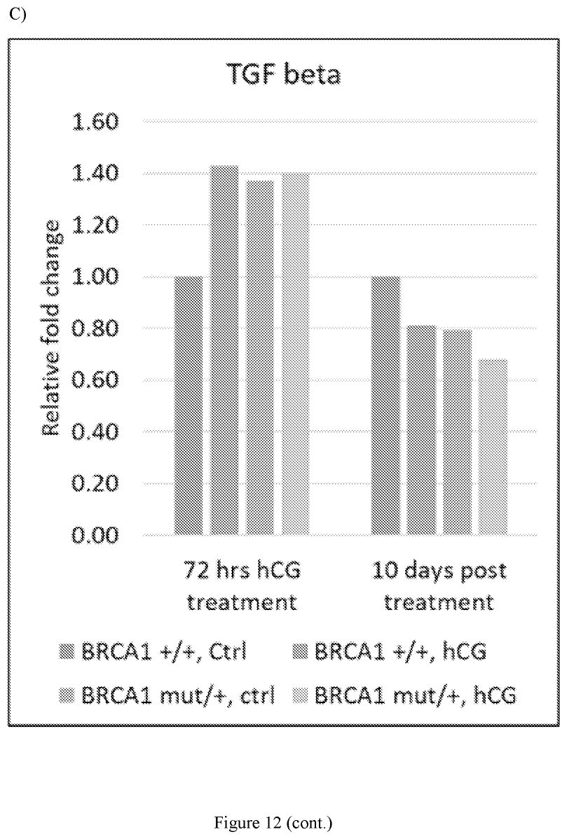

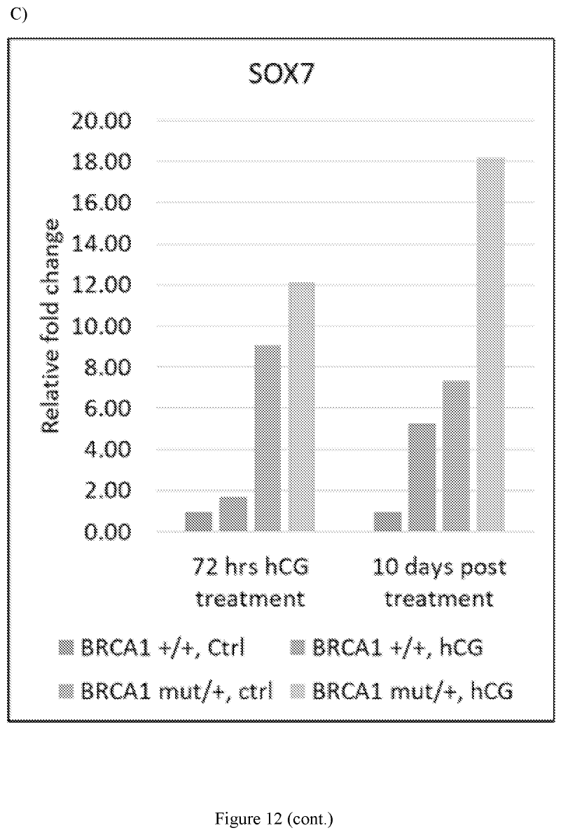

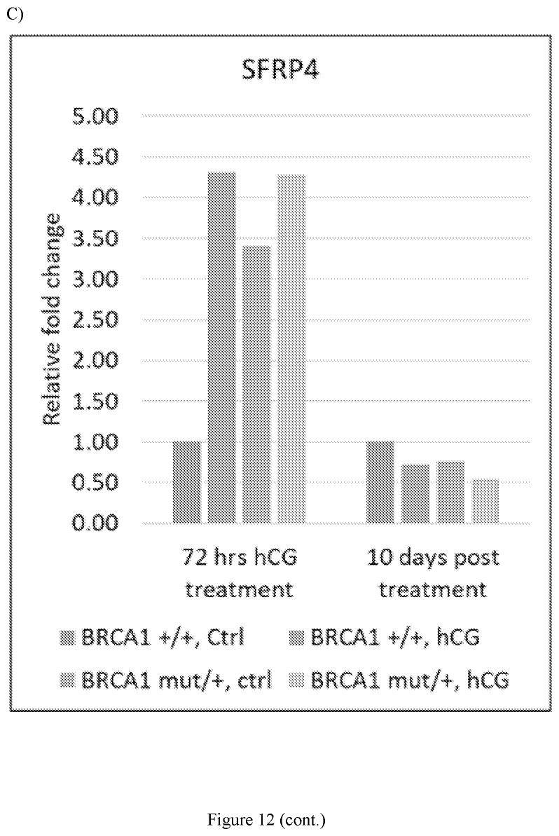

RNA-sequencing analysis of the breast tissues from rhCG treated BRCA1/2 carriers showed that WNT/β-catenin was inhibited while the TGFβ signaling, BRCA1, and p53 were activated by rhCG treatment. In addition, microarray analysis of the transcriptomic profile of mammospheres from rhCG treated rats also showed that WNT/β-catenin signaling was inhibited. The negative regulators of WNT signaling such as SOX7, SOX17, SOX18, as well as SFRP4 were up-regulated in mammospheres of rhCG treated rats and in the breast tissues of rhCG treated BRCA1/2 carriers, indicating that inhibition of WNT/β-catenin is a common event induced by rhCG both in human and rats. It was determined whether the up-regulation of BRCA1 in breast epithelial cells could be the result from the inhibition of WNT signaling and activation of TGFβ pathway. Thus, the expression of TGFβ, SOX7 and SFRP4 was evaluated by WB. As shown in FIG. 12C, the protein level of TGFβ was increased in BRCA1^{+/+} cells at the end of 72 hours rhCG treatment. The expression of TGFβ was not changed at the end of treatment, it might change earlier or later than the time point we evaluated. 10 days post rhCG treatment, TGFβ was slightly decreased in both BRCA1^{+/+} and BRCA1^{mut/+} cells. SOX7 level was increased in both cell lines at both the end of rhCG treatment and 10-days post treatment. SFRP4 level was increased at the end of 72 hours rhCG treatment. It was then determined whether there was a change in miR182 expression. Quantitative RT-CPR was performed by TaqMan miRNA assay (FIG. 12C). The results showed that miR182 was significantly reduced by rhCG treatment in both BRCA1^{+/+} and BRCA1^{mut/+} cell line at the time of 10 days post rhCG treatment (the analysis at other time points are ongoing). These data suggest that rhCG treatment might regulate BRCA1 and FOXO3A expression partly through activating TGFβ and inhibiting WNT signaling.

### RhCG Induces p53 Expression in Breast Epithelial Cells:

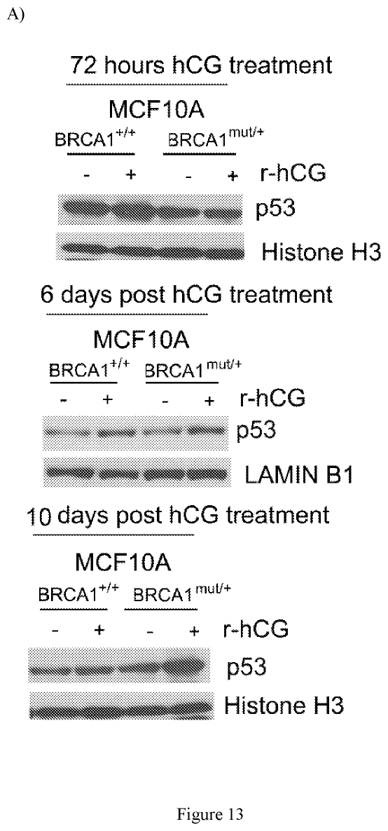

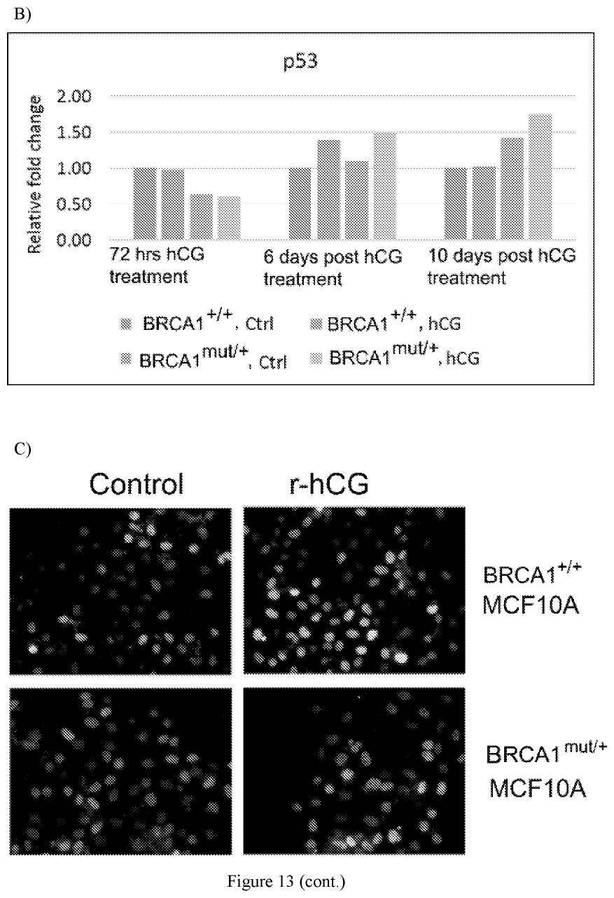

Tumor suppressor p53, which is encoded by TP53 in human, has been described as “the guardian of the genome” because of its functions in apoptosis and genome stability. The expression of p53 is higher (1.3 fold) in the breast of early parous women (first full term pregnancy=<25 yr) compared to nulliparous women. p53 interacts with a series of proteins, BRCA1 and BRCA2 are two of them. BRCA1 physically associates with p53 and stimulates its transcriptional activity. p53 protein was increased in both BRCA1 WT and mutation carrier MCF10A cells at 6 days and 10 days post rhCG treatment detected by WB (FIG. 13A and FIG. 13B). The immunofluorescence staining also detected the increase of p53 at the end of 72 hours treatment (FIG. 13C).

### RhCG Treatment Promotes DNA Repair in Breast Epithelial Cells:

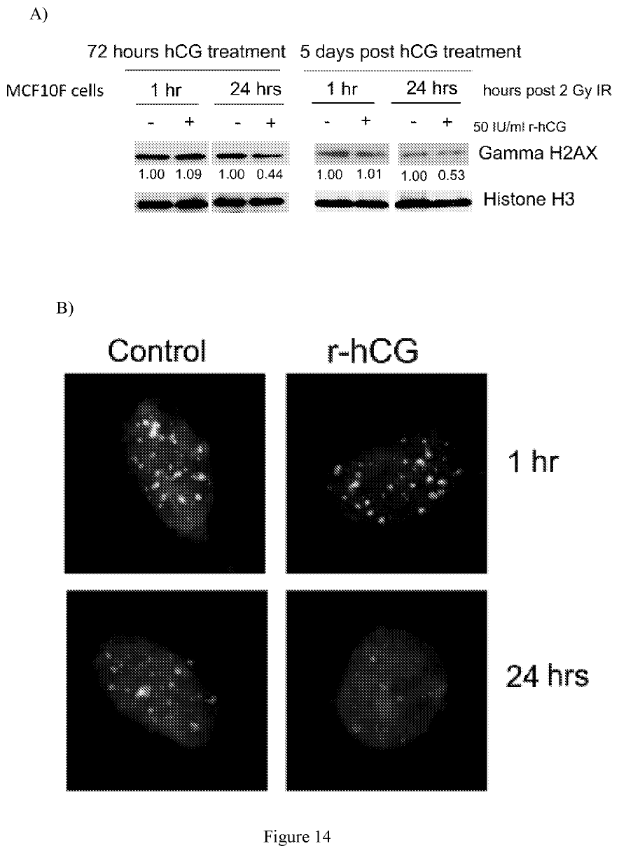

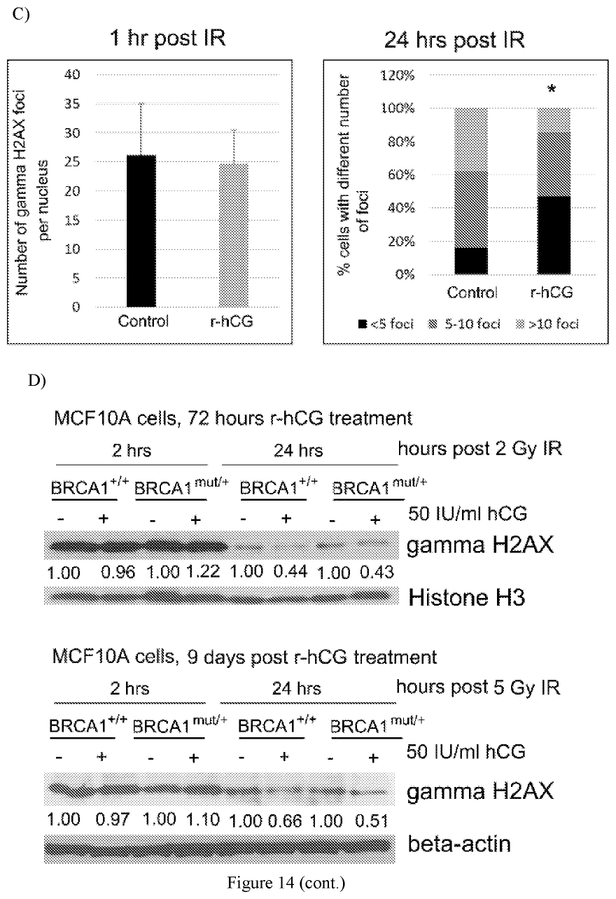

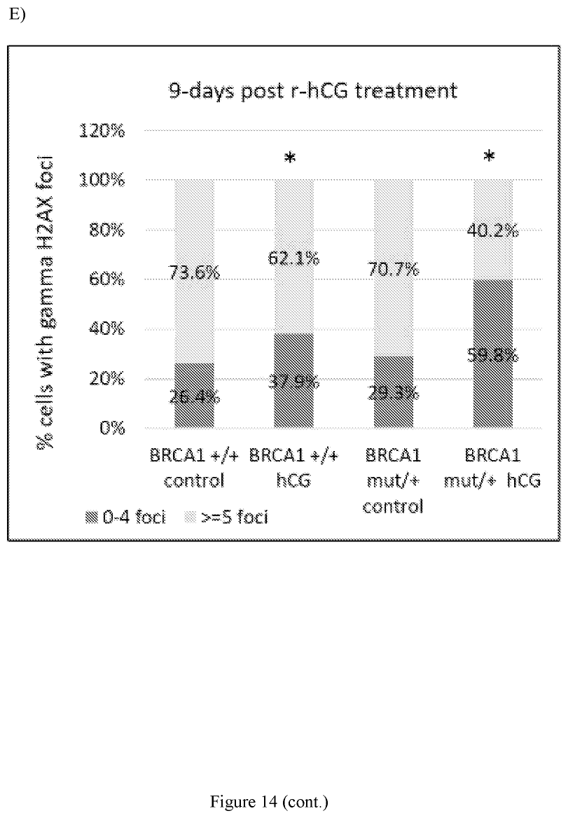

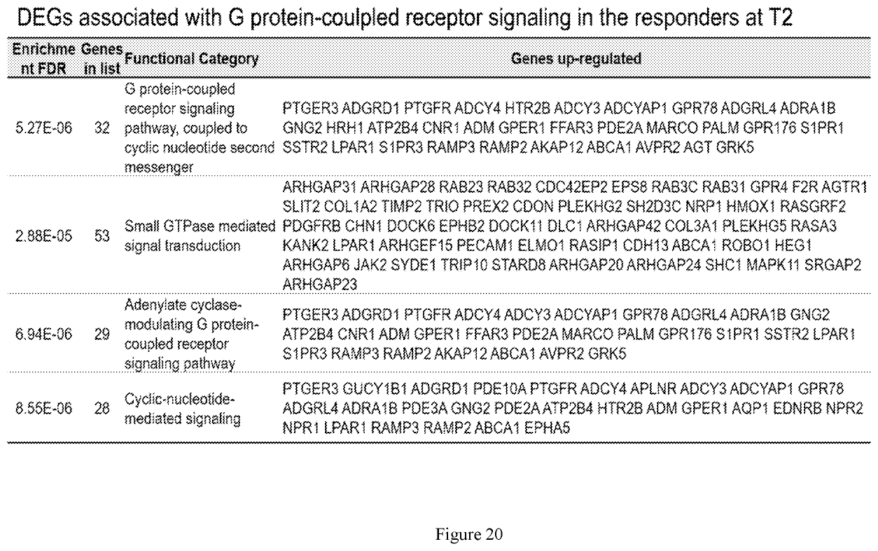

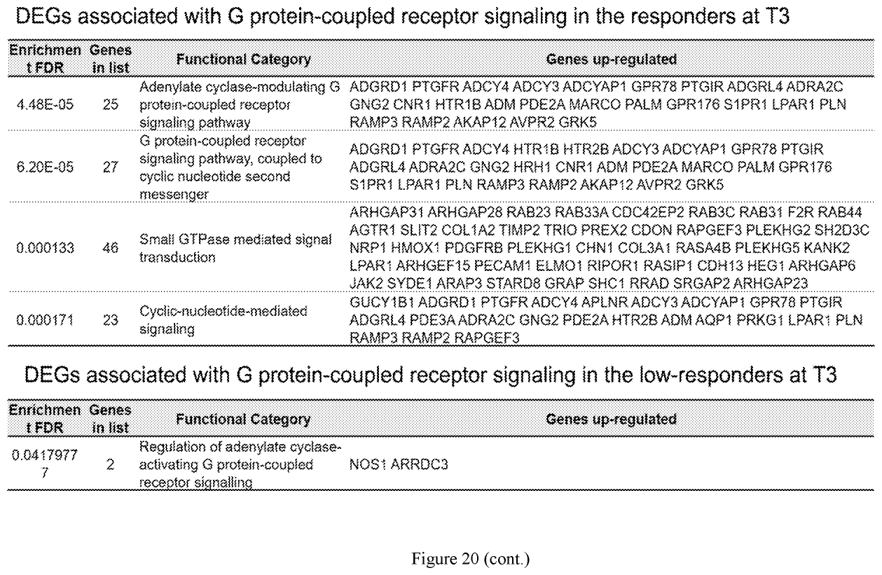

One of the most important functions of BRCA1 and p53 is DNA repair. The observation that BRCA1, BARD1, FOXO3A, and p53 are upregulated in the breast epithelial cells by rhCG treatment suggests that rhCG may have an important role in DNA repair. Therefore, MCF10F cells were treated with rhCG, and then cells were irradiated with 2 Gy gamma irradiation. DNA repair was evaluated by WB and immunofluorescence staining of DNA double strand breaks (DSB) with gamma H2AX antibody. The results showed that gamma H2AX level at 24 hours post gamma irradiation was decreased by 56% when cells were treated with rhCG before irradiation, although the gamma H2AX level was the same at 1-hour post irradiation. Importantly, this effect was also observed 5 days post rhCG treatment (FIG. 14A). Consistent with the decreased gamma H2AX level in total cell lysates of rhCG treated cells evaluated by WB, reduced number of gamma H2AX foci on the nuclei of cells treated with rhCG was observed by immunofluorescence staining of gamma H2AX (FIG. 20B and FIG. 14C). These data indicate that rhCG treatment promotes DNA repair in MCF10F cells, and the effect persists after the treatment stops. These results were further demonstrated in both BRCA1^{+/+} and BRCA1^{mut/+} MCF10A cells, even at 9-days post rhCG treatment and with 5 Gy gamma irradiation, rhCG treated cells still showed a decreased gamma H2AX level when compared with cells without rhCG treatment prior to gamma irradiation (FIG. 14D). Analysis of gamma H2AX foci 6 hours post 5 Gy gamma irradiation also demonstrated that even 9 days post rhCG treatment, these cells had an increased DNA repair compared to cells without rhCG treatment (FIG. 14E). Taken together, these results suggest that rhCG promotes DNA repair in both BRCA1 wild type and mutation carrier breast epithelial cells.

### RhCG Treatment Increases Histone H3 Tri-Methylation at Lysine 27 (H3K27Me3) in Mammary Epithelial Cells:

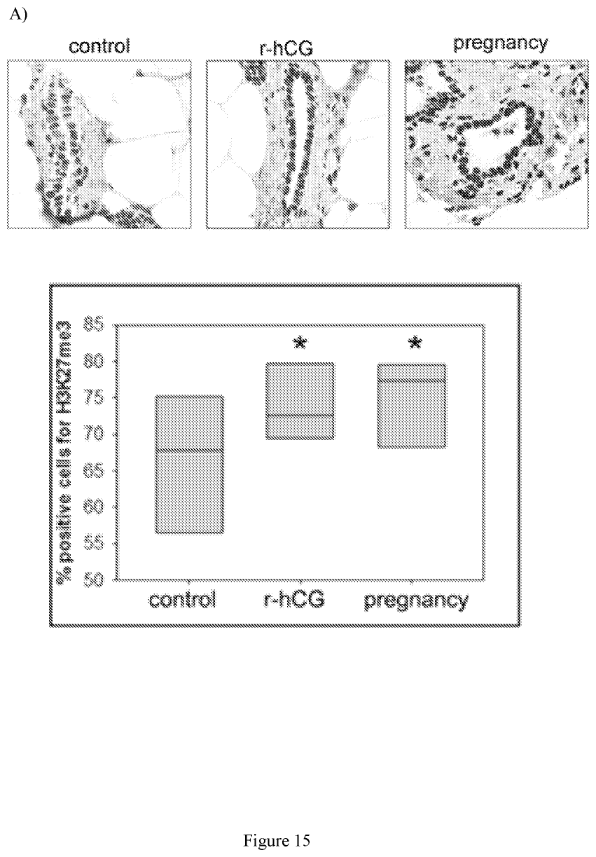

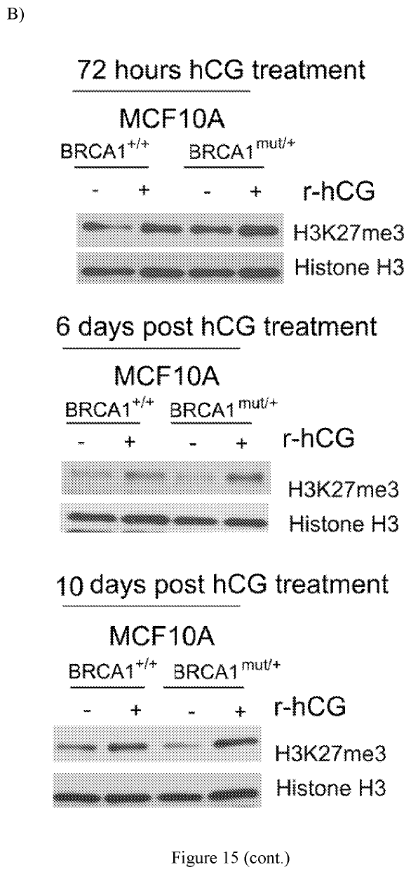

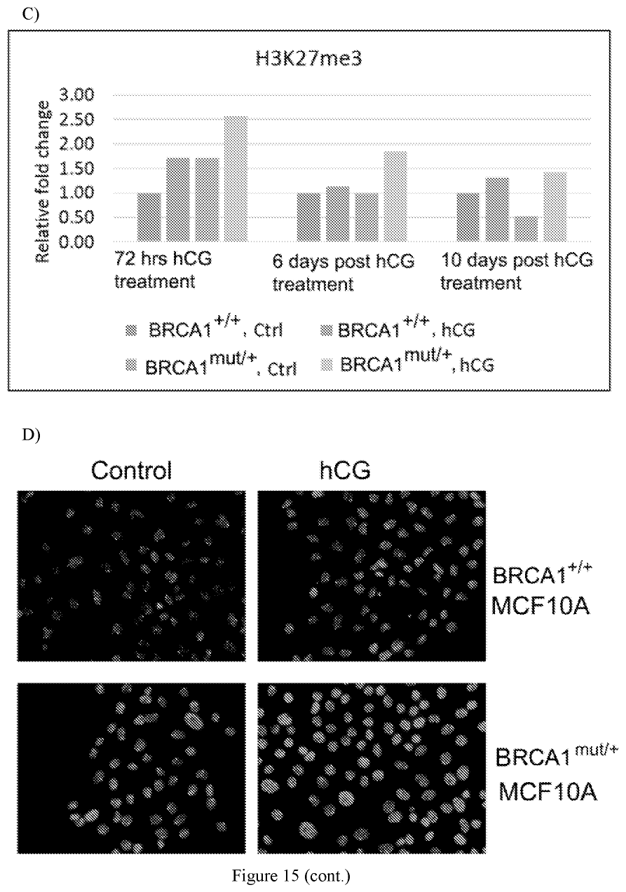

The development of mammary gland is a lifelong process initiated during embryonic life and proceeds postnatal through puberty, pregnancy, lactation, and involution. The mammary epigenome undergoes specific change and plays important roles in regulating cell-fate during the development. Correlating the global H3K27me3 modification maps with gene expression signatures indicated that the epigenome has an important role in directing cell-fate. The number of genes showing enriched H3K27me3 occupancy at transcription start site (TSS) increased upon luminal lineage specification compared to mammary stem cell subset. Moreover, the mammary epigenome was highly sensitive to hormonal environments, the total number of genes within the luminal subset with significant H3K27me3 modifications relative to input increased during pregnancy. H3K27me3 emerged as a key mediator of gene expression changes during pregnancy. The breast epithelial cells of postmenopausal parous women exhibit an increased H3K27me3 compared to that of nulliparous women. When the H3K27me3 level in the rat mammary gland epithelial cells was evaluated by immunohistochemistry, the global H3K27me3 level and the number of cells positive for H3K27me3 was increased in rat mammary gland 15-days post rhCG treatment, at a level similar to that in the mammary gland of 15 days post-delivery (FIG. 15A).

It was determined whether the increase of H3K27me3 is a direct effect of hCG on mammary epithelial cells, or whether it is a systemic effect through other organs or hormones in vivo. Thus, MCF10A cells were treated and the H3K27me3 level was determined by WB. Consistently, H3K27me3 was increased in both BRCA1^{+/+} or BRCA1^{mut/+} cells at the time of finishing 72 hours rhCG treatment, and 6 days or 10-days post rhCG treatment (FIG. 15B, FIG. 15C, and FIG. 15D), suggesting rhCG mimics pregnancy and has a direct role in chromatin remolding in mammary epithelial cells.

The data from this study supported that rhCG has a direct role in regulating the expression of tumor suppressors BRCA1, BARD1, FOXO3A, and p53 in mammary epithelia cells, consistent with the observation that rhCG induced BRCA1 and FOXO3A expression and activating BRCA1 and p53 in the breast epithelial cells of BRCA1/2 carriers after rhCG treatment. The regulation of rhCG on BRCA1 expression might be partly through down-regulating miR182 by activating TGFβ signaling and inhibiting WNT/β-catenin signaling. rhCG treatment promotes DNA repair in breast epithelial cells, suggesting a cancer prevention role through up-regulating BRCA1, p53 and other genes related to DNA repair. rhCG induces chromating remodeling, which is consistent with the findings that there was a higher level of global H3K27me3 in the breast epithelial cells of parous postmenopausal women.

### Example 6: Transcriptomic Analysis of Mammospheres Generated from Mammary Epithelial Cells of rhCG Treated Rats Supports that rhCG Induces Cell Differentiation and Inhibiting Wnt/β-Catenin Signaling

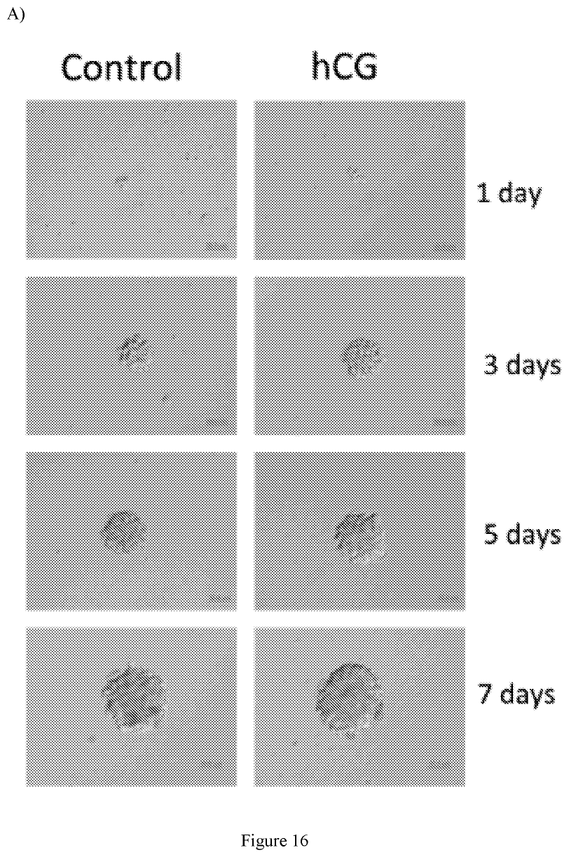

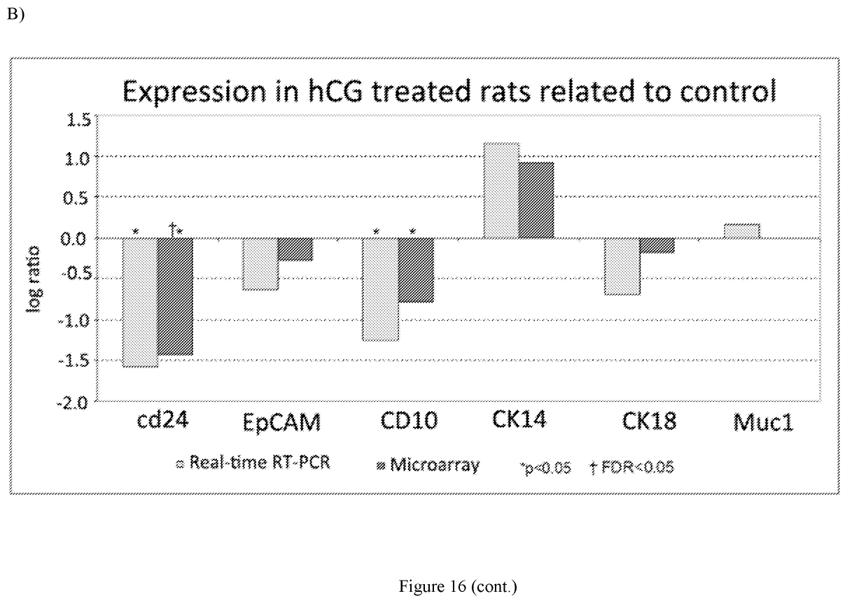

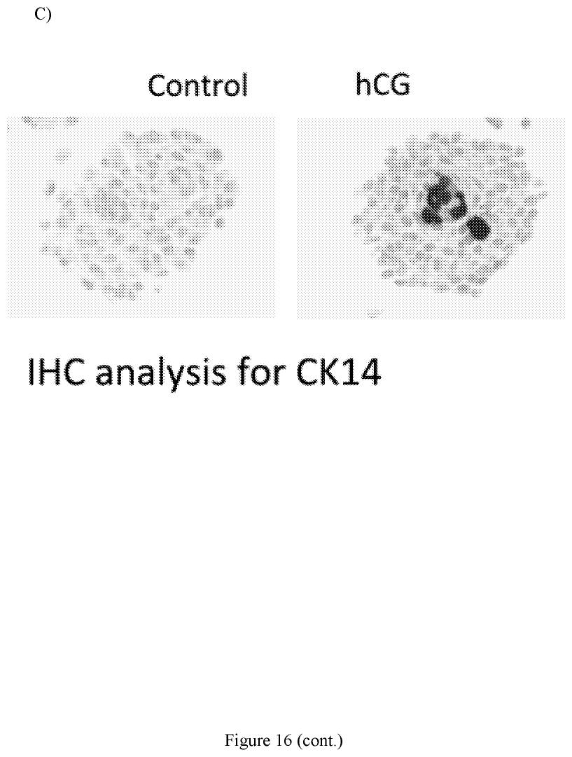

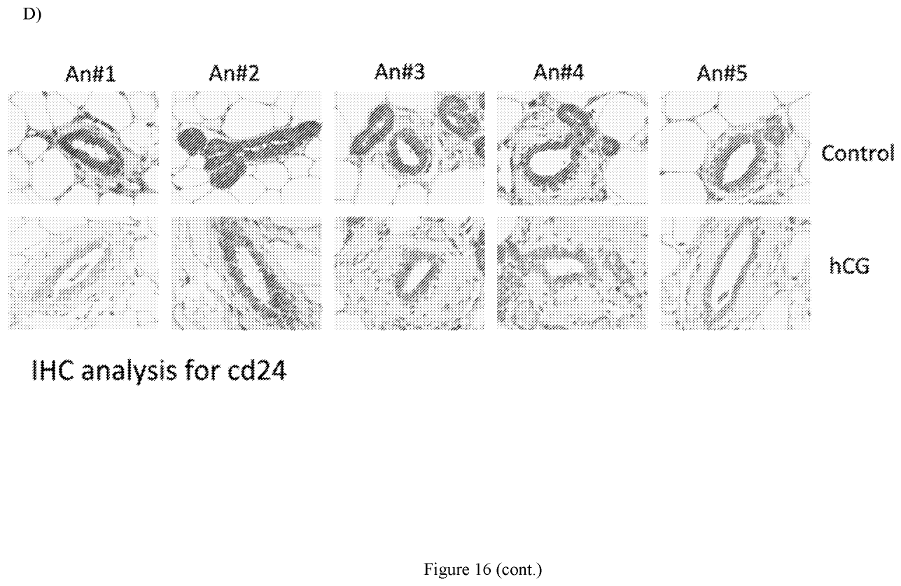

It was hypothesized that rhCG has an effect on mammary stem cells based on its effect on inducing mammary gland differentiation and suppressing mammary tumorigenesis after DMBA challenge. Thus, 55 day old Sprague-Dawley rats were treated with rhCG at the dose of 100 IU/rat/day for 3 weeks, then rat mammary epithelial cells were isolated 21-days post rhCG treatment using EasySep™ Mouse Epithelial Cell Enrichment Kit (Stemcell Technologies, Cambridge, MA). The mammary epithelial cells formed mammospheres when cultured in EpiCul™-B Mouse Medium Kit (Stemcell Technologies, Cambridge, MA). The frequency of primary mammospheres formed from these cells was 2-6 spheres/1000 cells. The number of observed primary mammospheres (representative images of mammospheres are shown in FIG. 16A) generated from mammary epithelial cells of rats 21-days post rhCG treatment was significantly reduced when compared with that from control rats (56.6±4.0 mammospheres for control, 37.2±2.0 for rhCG group, n=3, ttest p=0.002), suggesting that rhCG treatment decreases the mammary stem cell population.

Total RNA was extracted from the primary mammospheres, and microarray was performed using whole genome Agilent Microarrays of rat containing about 41,000 probes representing about 19,000 unique gene symbol. When using FDR 5 and fold change 2, there were 149 differentially expressed genes (DEGs; 49 genes upregulated in rhCG group, and 100 genes down-regulated). The GOs with the most DEGs are system development, negative regulation of cellular process, biology regulation, and signaling. Analysis of canonical pathways enriched by upregulated genes is Wnt/β-catenin signaling, and pathways enriched by down-regulated genes are immune related pathways (Table 8). It is important to note that the genes upregulated in Wnt/β-catenin signaling such as SOX7, SOX17, SOX18, SFRP4 are negative regulators of Wnt/β-catenin signaling, and WNT2 is down-regulated by rhCG, further demonstrating that rhCG inhibits Wnt/β-catenin signaling not only in the breasts of parous postmenopausal women and rhCG treated BRCA1/2 carriers, but also in the mammary glands of parous mouse and rhCG treated rats. A heat map of transcription factors among DEGs with p<0.01 and absolute fold change 2 was prepared (data not shown).

Selected genes related to mammary gland development were validated by real-time RT-PCR and immunohistochemical (IHC) analysis. Cd24 and CD10 are both significantly down-regulated by Microarray and RT-PCR analysis (FIG. 16B). IHC staining of rat mammary gland also showed cd24 was reduced significantly (p=0.05) in mammary epithelial cells of rhCG treated rats (FIG. 16D). Cd24 is a surface marker usually used to isolate mammary stem cells from mouse, Lin⁻cd24⁺cd29^{high} mammary epithelial cells consist mammary stem cells capable of generating a functional mammary gland when transplanted in clear mouse mammary fat pad. CD10 is a zinc-dependent metalloprotease that regulates the growth of the ductal tree during mammary gland development. CD10^{high}EpCAM^{-/low} population is enriched for early common progenitor and mammosphere-forming cells Down-regulation of cd24 and CD10 in mammospheres of rhCG treated rats suggest that rhCG treatment reduces stemness of mammary stem/progenitor cells. CK14 is indicative of more mature mammary epithelial cells The expression of CK14 is significantly increased in mammospheres (FIG. 16C) from rhCG treated rats (35±3.5% of mammospheres are positive in hCG group whereas only 18±3.8% are positive for control, T test p=0.0088), suggesting rhCG induces mammary stem cells differentiation. Another interesting finding is the upregulation of TGFβ1 by microarray in the mammospheres of rhCG treated rats, which is consistent with the findings that TGFβ1 is activated in the breast tissues of rhCG treated BRCA1/2 carrier women. In summary, rhCG treatment induces inhibition of Wnt/β-catenin signaling and activation of TGFβ, and reduces stemness in the mammary glands of both human and rats.

TABLE 8

| | | | |
|---|---|---|---|
| Ingenuity Canonical Pathways | p-value | Ratio | Molecules |

Canonical Pathways enriched (p < 0.01) by up
regulated genes (FDR 5% and Fold Change 2.0)

| | | | |
|---|---|---|---|
| Wnt/Î²-catenin Signaling | 2.00E−03 | 0.02 | SOX7, |
| | | | SOX17, SFRP4, CDH5, SOX18 |

Canonical Pathways enriched (p < 0.01) by down
regulated genes (FDR 5% and Fold Change 2.0)

| | | | |
|---|---|---|---|
| Graft-versus-Host Disease | 6.03E−06 | 0.09 | IL1A, HLA-DRA, HLA- |
| Signaling | | | DQA1, HLA-C |
| OX40 Signaling Pathway | 2.88E−05 | 0.05 | TNFSF4, HLA-DRA, HLA- |
| | | | DQA1, HLA-C |
| Communication between Innate | 5.37E−05 | 0.04 | IL1A, HLA-DRA, CD83, HLA-C |
| and Adaptive Immune Cells | | | |
| Dendritic Cell Maturation | 1.07E−04 | 0.03 | IL1A, HLA-DRA, HLA- |
| | | | DQA1, CD83, HLA-C |
| Antigen Presentation Pathway | 1.29E−04 | 0.07 | HLA-DRA, HLA-DQA1, HLA-C |
| Autoimmune Thyroid Disease | 2.14E−04 | 0.06 | HLA-DRA, HLA-DQA1, HLA-C |
| Signaling | | | |
| Allograft Rejection Signaling | 3.24E−04 | 0.04 | HLA-DRA, HLA-DQA1, HLA-C |
| Cytotoxic T Lymphocyte-mediated | 5.01E−04 | 0.04 | HLA-DRA, HLA-DQA1, HLA-C |
| Apoptosis of Target Cells | | | |
| B Cell Development | 1.66E−03 | 0.06 | HLA-DRA, HLA-DQA1 |
| Altered T Cell and B Cell Signaling | 1.66E−03 | 0.03 | IL1A, HLA-DRA, HLA-DQA1 |
| in Rheumatoid Arthritis | | | |
| Aryl Hydrocarbon Receptor | 1.91E−03 | 0.03 | CDKN2A, IL1A, NQO1, CYP1B1 |
| Signaling | | | |
| Crosstalk between Dendritic Cells | 2.24E−03 | 0.03 | HLA-DRA, CD83, HLA-C |
| and Natural Killer Cells | | | |
| Role of Cytokines in Mediating | 5.62E−03 | 0.04 | IL1A, CSF3 |
| Communication between Immune | | | |
| Cells | | | |
| Type I Diabetes Mellitus Signaling | 7.41E−03 | 0.03 | HLA-DRA, HLA-DQA1, HLA-C |
| Cdc42 Signaling | 9.12E−03 | 0.02 | HLA-DRA, HLA-DQA1, HLA-C |

### Example 7: R-hCG Treatment Induces Remarkable Transcriptomic Changes in the Breast Tissue of BRCA1/2 Carriers

To identify the transcriptomic changes induced by r-hCG, RNA-seq was performed on breast tissues from 25 women. The analysis showed that the response to r-hCG treatment was not associated with the BRCA1 or BRCA2 status, but strikingly related to the use of hormonal contraceptives during the clinical trial.

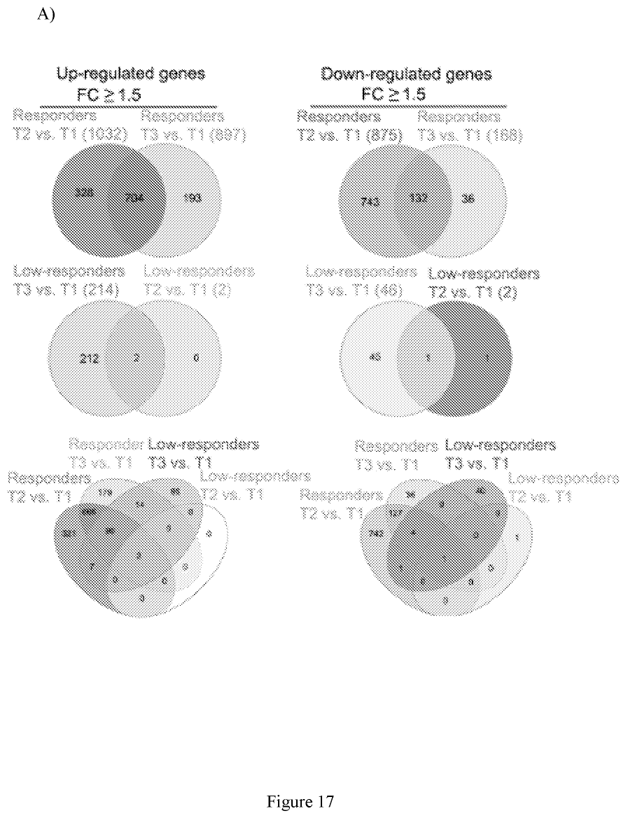

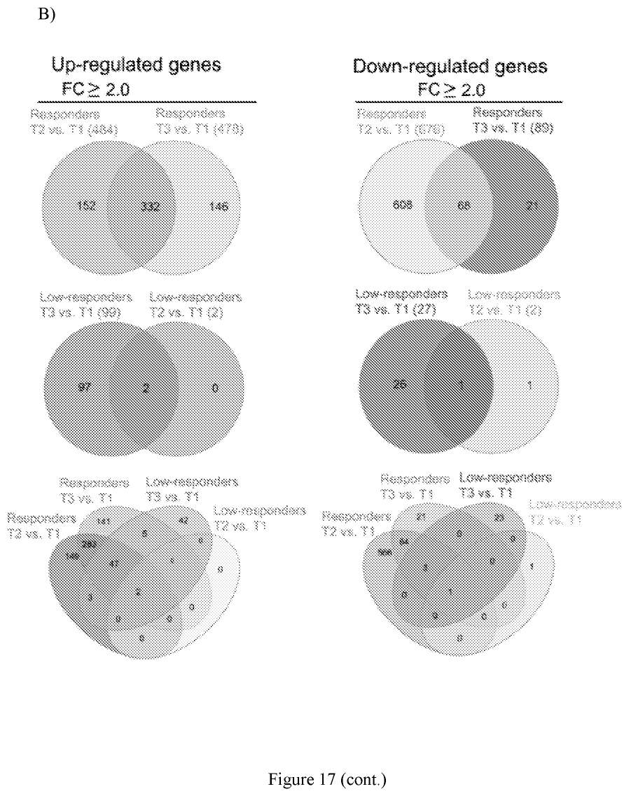

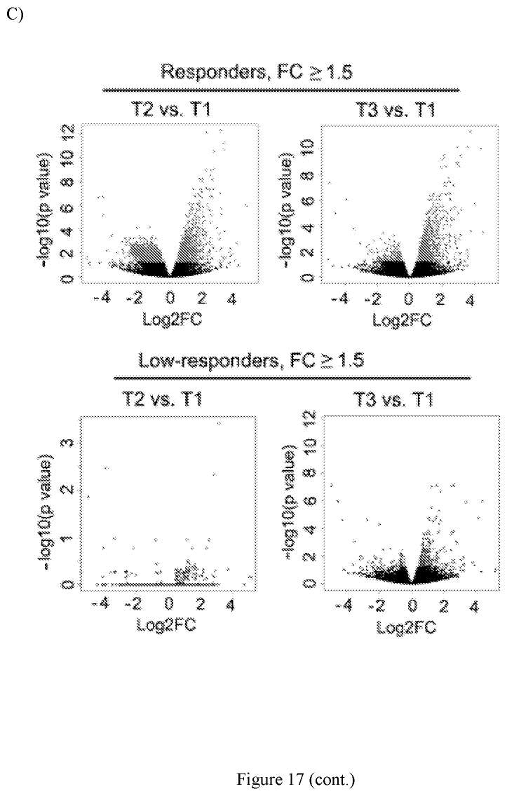

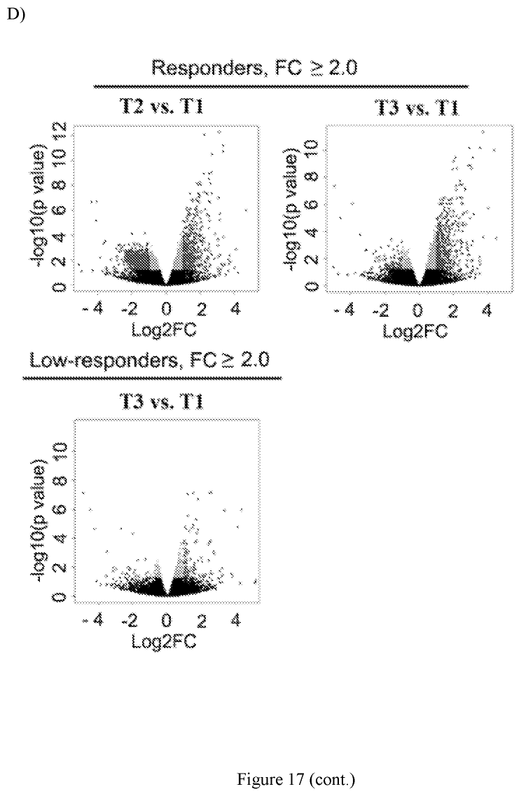

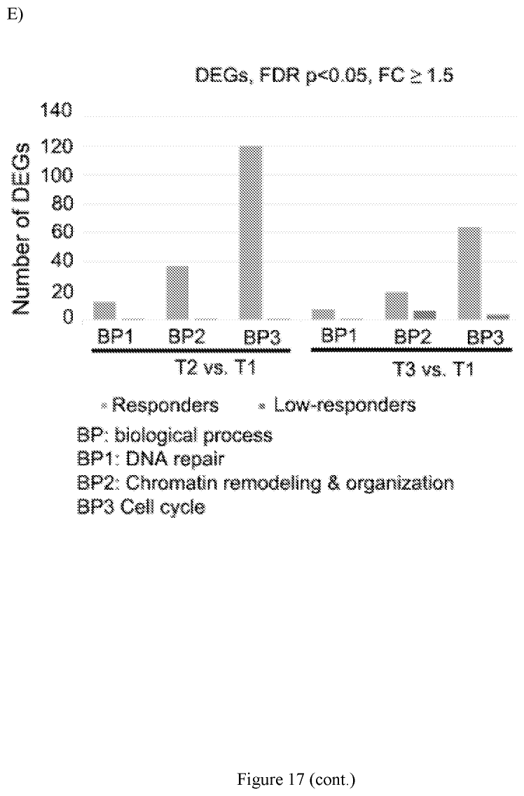

Venn Diagrams (FIG. 17, Panel A and Panel B) show the number of DEGs with cutoff fold change of 1.5 and 2.0 (FC1.5 and FC2). There were 1907 DEGs at T2 and 1065 DEGs at T3 for 11 women without contraceptives (named as responders) while there was almost no response at T2 and only 260 DEGs at T3 for 14 women with contraceptives (named as low-responders) using cutoff FC1.5 (data not shown). In addition, there were some common up-regulated DEGs between responders and low-responders, whereas the down-regulated genes were very different, suggesting contraceptives resulted in a delayed and reduced response to r-hCG, and might induce a distinct effect.

Both volcano plots (FIG. 17, Panel C and Panel D) and heatmap (data not shown) clearly show a large portion of down-regulated DEGs at T2, and a great number of up-regulated DEGs at both T2 and T3 in responders, indicating the persistent and prolonged effect of r-hCG on the transcriptomic profile. For low-responders, gene expression changes were only observed at T3 (data not shown), further confirmed the postponed and decreased impact of r-hCG.

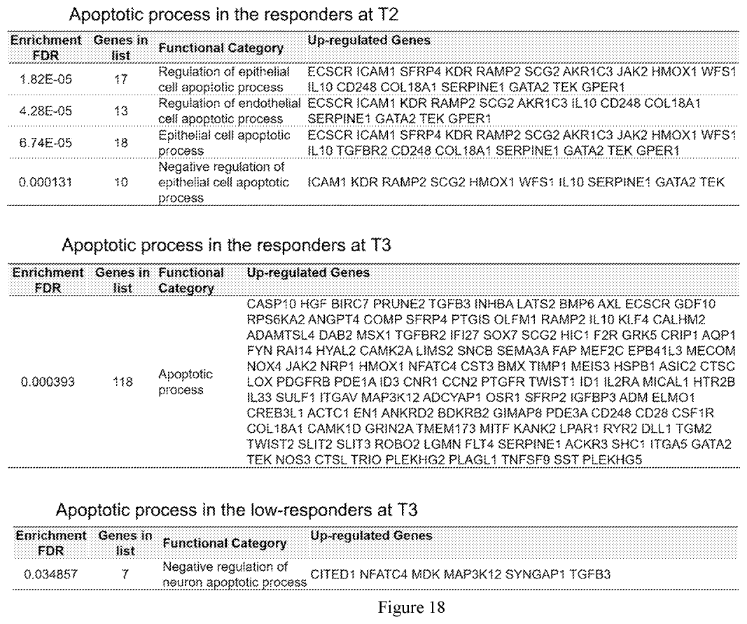

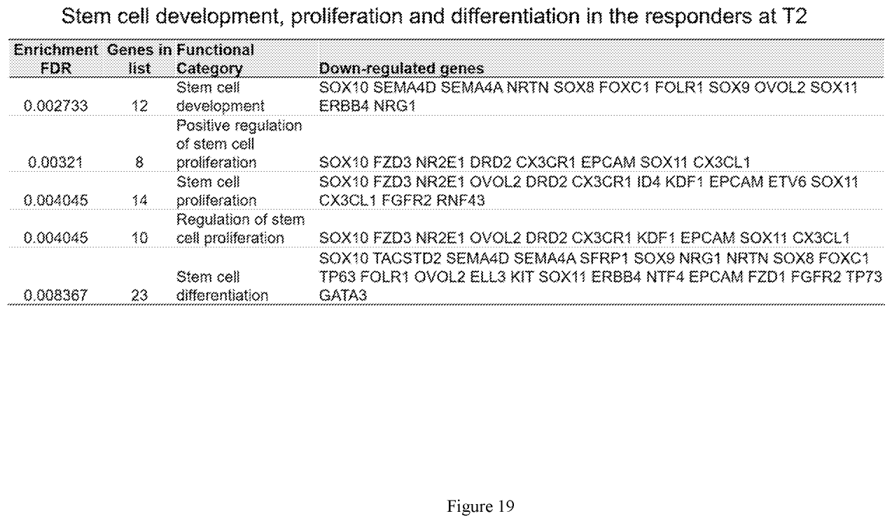

GO enrichment analysis revealed that r-hCG greatly affected cellular developmental process, cell differentiation, and anatomic structure morphogenesis at both T2 and T3 in responders and at T3 in low-responders (data not shown). Furthermore, DEGs related to cell cycle and apoptotic process were mainly observed in responders (FIG. 17, Panel E; FIG. 18). Notably, the processes of stem cell development, proliferation, and differentiation were observed at T2 in responders only and were down-regulated by r-hCG (FIG. 19). Some genes including KIT, NRG1, and SEMA4D in these processes are key regulators of stem cells. Reactome pathway analysis showed extracellular matrix organization and collagen formation were enriched with up-regulated genes in both responders and low-responders. Signal transduction, the top pathway of up-regulated DEGs at both T2 and T3, and post-translational protein modification that has 85 up-regulated DEGs at T2 in responders, were not found in low-responders. In addition, signaling by ERB13B2 and ERB13B4 were down-regulated in responders at T2 (data not shown), implying the impact of r-hCG on the prevention of ERBBs related tumor.

In summary, r-hCG has a remarkable effect on the transcriptomic profile of breast tissue from BRCA1/2 carriers who did not use contraceptives, whereas the use of contraceptives interfered with hCG's effects, delayed the response, and dramatically reduced the number of DEGs.

### Example 8: R-hCG Induces Expression Changes in Genes Related to DNA Repair, Chromatin Organization and Remodeling, and GPCR Only in Women without Contraceptive Exposure

A large number of DEGs associated with DNA repair, chromatin remodeling and organization at both T2 and T3 (FIG. 17, Panel E) were identified only in responders. These DEGs mainly affected chromatin modification, organization, and remodeling, transcription, cell differentiation, cell cycle, apoptosis, double-strand break repair, DNA replication, etc. at T2 (data not shown). Among them, HMGA1, MYC, PADI2, PADI3, and SOX9 were also related to other important pathways. HMGA1 encodes one of the most abundant non-histone chromatin remodeling proteins. HMGA1 transcriptional networks involve all hallmarks of cancer (Sumter et al., Curr. Mol. Med., 2016, 16, 353-93). At T3, these DEGs were involved in not only the processes observed at T2 but also tissue development (data not shown).

In the present study, 75 genes related to GPCR signaling were up-regulated at T2 and/or T3 in responders compared to only 2 up-regulated genes at T3 in low-responders (FIG. 20). A more detailed analysis was run to inspect differences in these DEGs between responders and low-responders (data not shown). A general tendency to up-regulation relative to the expression at T1 was identified both for responders and low-responders, whereas the changes in responders were more striking and significant. The use of contraceptives was associated with slightly higher initial gene expression (T1) in low-responders, and then to lower increases (T2 vs. T1 and T3 vs. T1) in this group compared to responders. The results suggest that in responders GPCR signaling was strongly and immediately activated under the effect of r-hCG, the use of contraceptives might delay and interfere with GPCR signaling via its influence on the initial expression of GPCR related genes or binding of r-hCG with its receptor.

### Example 9: R-hCG Treatment Inhibits Wnt/β-Catenin Canonical Pathway in the Breast Tissue of BRCA1/2 Carriers

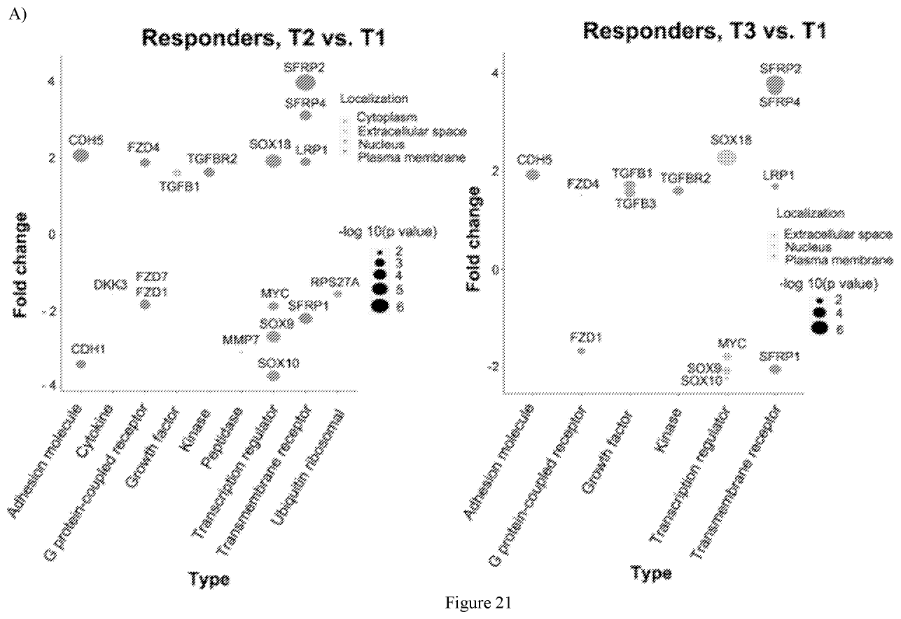

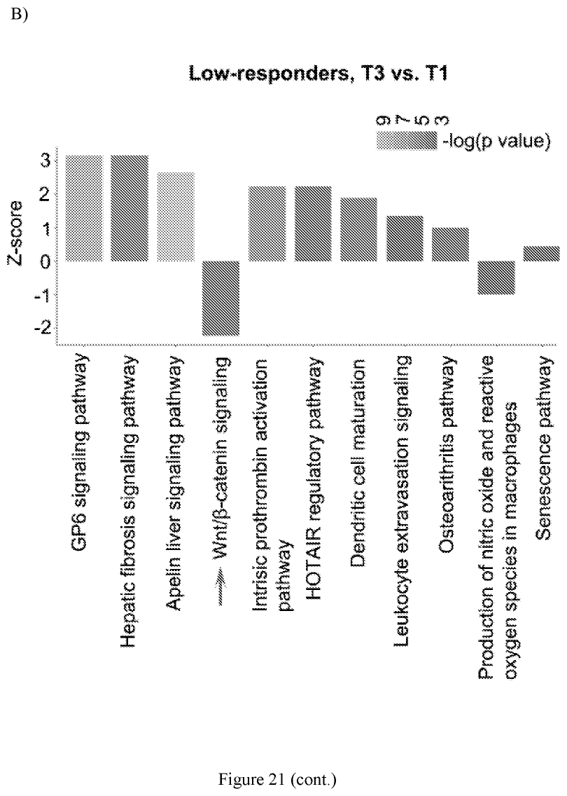

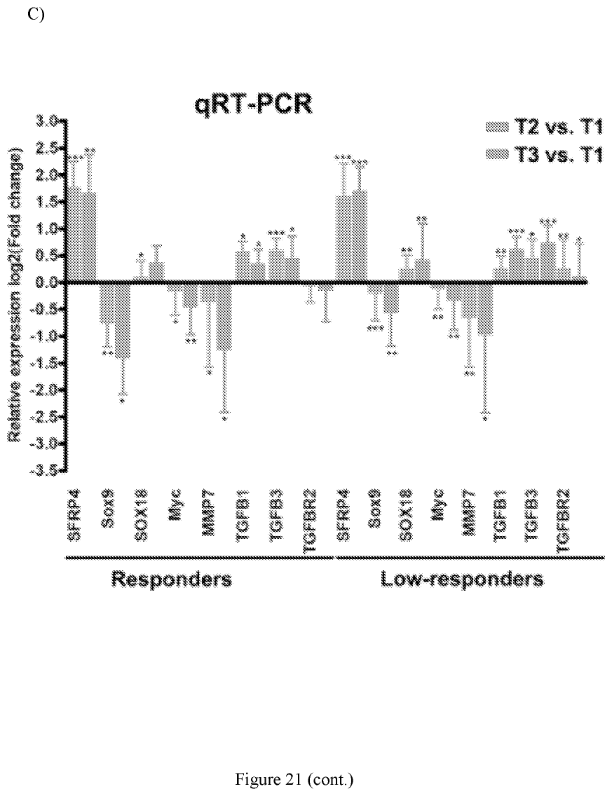

Ingenuity Pathway Analysis (IPA) was performed to identify the enriched canonical pathways of the DEGs. Activation or inhibition of many pathways that are implicated in development and tumorigenesis was observed, of which, Wnt/β-catenin and PPAR signaling pathway were inhibited while p38 MAPK signaling and cAMP-mediated signaling were activated in responders at both T2 and T3 (data not shown). In addition, ErbB2-ErbB3 signaling, Wnt/Ca+ pathway, and mouse embryonic stem cell pluripotency were inhibited whereas prolactin signaling was activated at T2, and TGFβ signaling was activated at T3 in responders. For the network of Wnt/β-catenin signaling, positive regulators including SOXE family (SOX9, SOX10) and frizzled receptors (FZD1, FZD7) were down-regulated, while negative regulators including SOXF family (SOX7, SOX17, and SOX18) and SFRP family (SFRP2, SFRP4) were up-regulated (FIG. 21, Panel A). IPA depicted the DEGs involved in canonical Wnt/β-catenin signaling at T3 in responders (data not shown). A similar change was observed in low-responders at T3 with the up-regulation of SFRP2, SFRP4 and SOX18 to a less extent, also resulting in Wnt/β-catenin signaling inhibition (FIG. 21, Panel B). Validation by qRT-PCR (FIG. 21, Panel C) confirmed the changes of selected genes in Wnt signaling. Overall, the results strongly indicate that r-hCG treatment inhibited Wnt/β-catenin signaling pathway in the breast of the responders both at the end of r-hCG treatment and six months later. Whereas in low-responders, the inhibition was delayed, and the extent of inhibition was decreased too.

### Example 10: R-hCG Treatment Activates Upstream Regulators TGFB/TGFBR-SMAD2/3/4, TP53 and BRCA1, Whereas Inhibits MYC, and Induces BRCA1 Protein in the Breast of BRCA1/2 Carriers

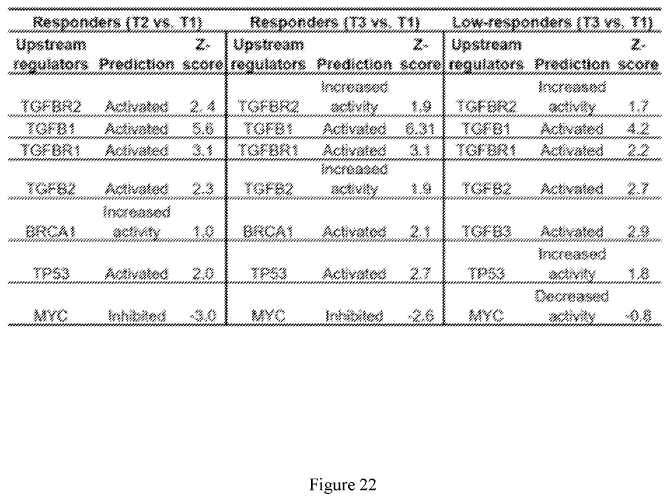

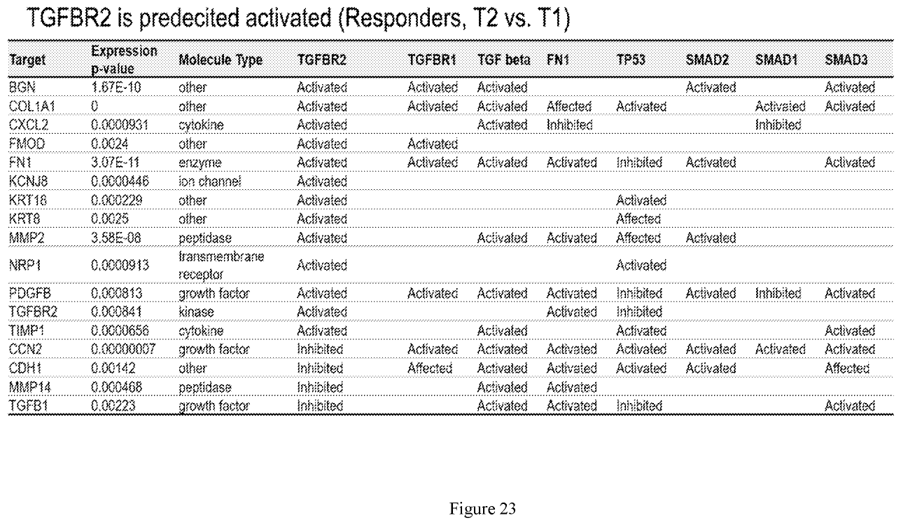

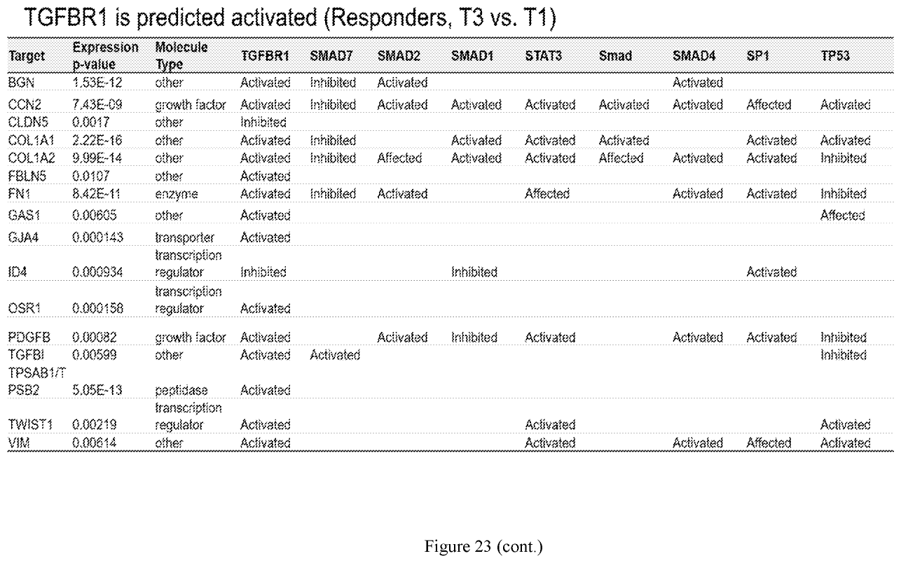

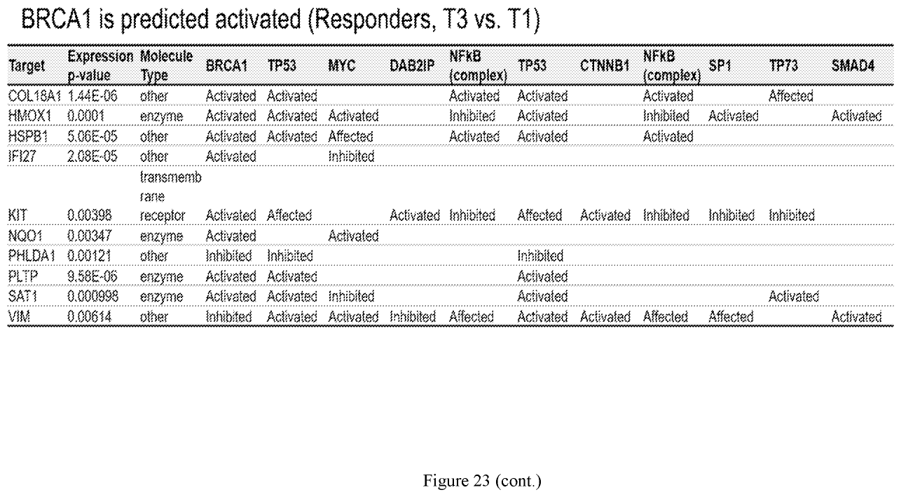

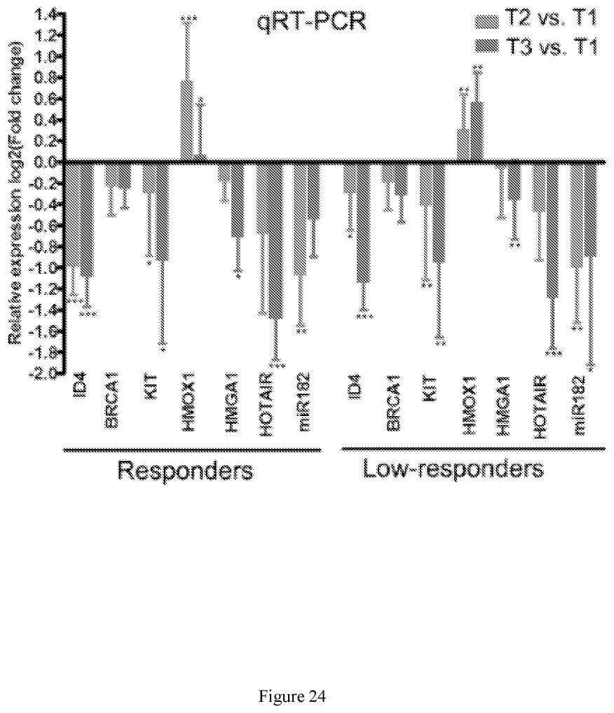

Upstream regulator analysis was performed and eight upstream regulators that are related to breast development and carcinogenesis and have the highest absolute Z-score were selected. It was identified that TGFB1, TGFBR1, and TP53 were predicted activated whereas MYC was strongly inhibited at T2 and T3 in responders (FIG. 22 and FIG. 23). Moreover, TGFB2 and TGFBR2 were activated at T2 and still had an increased activity at T3 in responders. There was a similar impact at T3 to these regulators in low-responders with a lower Z-score except for TGFB2 and TGFB3. Notably, BRCA1 was predicted activated at T3 in responders only. IPA revealed that the number of DEGs as target genes of the upstream regulators in responders was much greater than that in low-responders (data not shown). In addition, consistent with the activation of TGFBR1/2, SMAD2/3/4 were predicted activated over time in both groups, while down-regulation of HMGA1, a target gene of MYC, was observed in responders only. Chord diagrams show the relationship between regulators and target DEGs. The expression changes of ID4 (TGFBR1 target), KIT (BRCA1 target), HMOX1 (BRCA1 and TP53 target), and HMGA1 (MYC target) were confirmed by qRT-PCR (FIG. 24). It was determined that the expression of long non-coding RNA HOTAIR, a MYC-activated driver of malignancy implicated in breast carcinogenesis (Mozdarani et al., J. Transl. Med., 2020, 18, 152). Consistently, HOTAIR was significantly down-regulated at T3 in 9/9 (100%) responders and 11/14 (78.6%) low-responders, suggesting the inhibition of MYC.

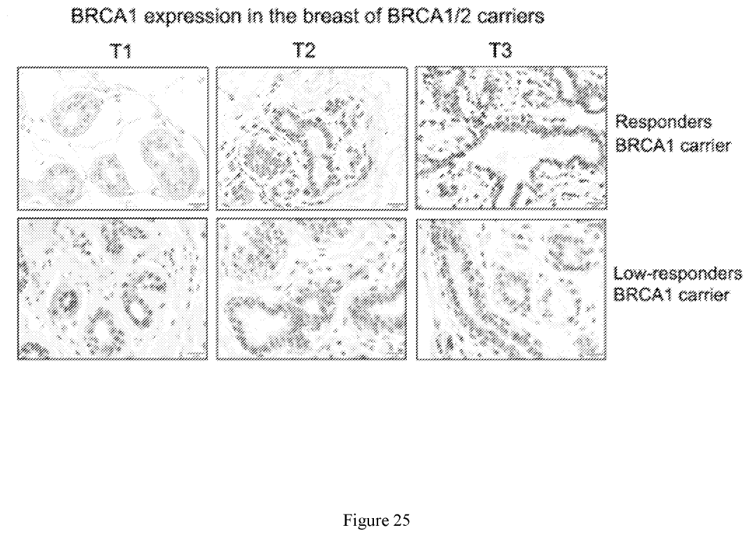

The expression of miR182 and BRCA1 was examined by qRT-PCR. There was no significant change in BRCA1 (using primers located on exons 22-23) although miR182 was significantly decreased at T2 in both groups and T3 in low-responders (FIG. 24). IHC was then performed on breast tissues with an antibody recognizing the N-terminal BRCA1 and detecting total BRCA1 protein since it is not possible to distinguish the wild type BRCA1 protein from mutant protein. Consistent with the finding that BRCA1 was activated at T3 in responders, r-hCG treatment significantly induced total BRCA1 protein at T3 in both BRCA1 and BRCA2 carriers only in responders (FIG. 25).

Taken together, the findings strongly suggest that r-hCG significantly activates TGFB/TFGBR-SMAD2/3/4 and TP53, whereas inhibits oncogene MYC and its target genes HMGA1 and HOTAIR in the responders. These effects were reduced and delayed in the low-responders. Additionally, r-hCG activates BRCA1 in the responders only, and induces BRCA1 protein expression might partially through TGFβ-miR182-BRCA1 axis.

### Example 11: R-hCG Treatment Suppresses Stemness and Inhibits Wnt/β-Catenin Signaling in Rat Mammary Epithelial Cells

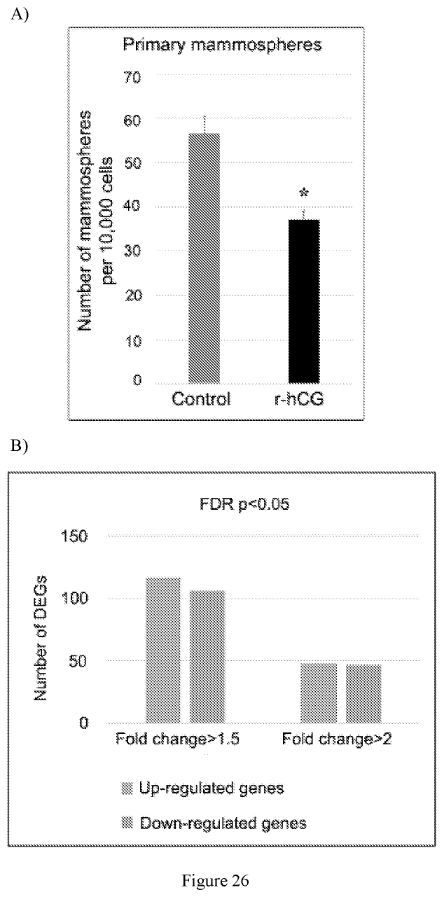

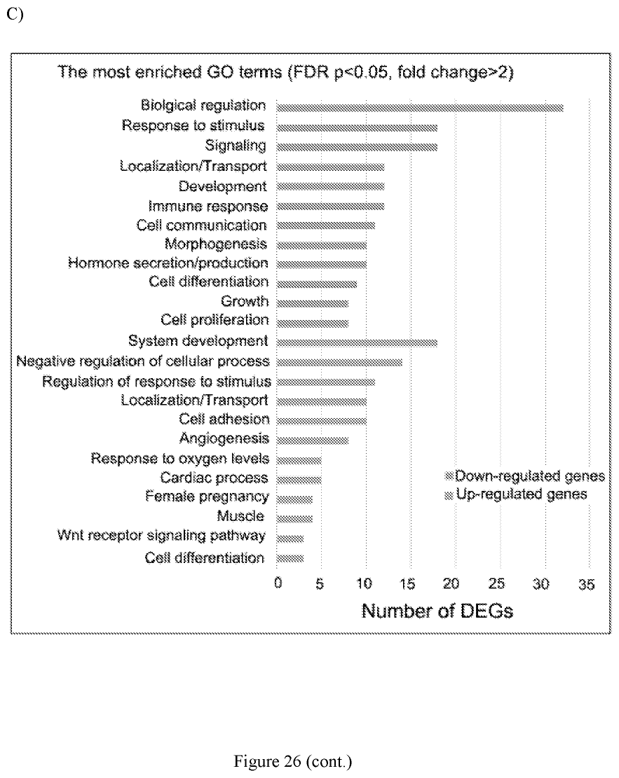

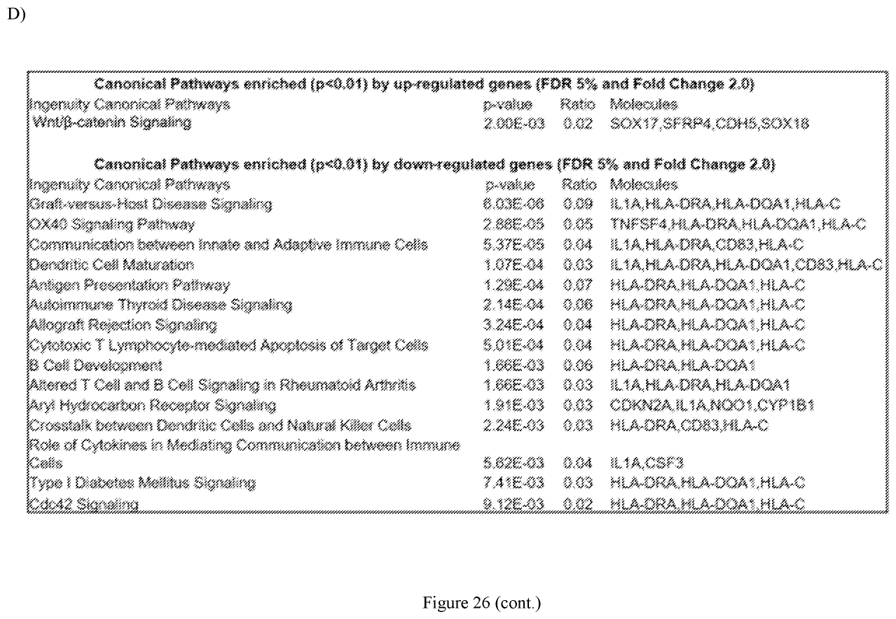

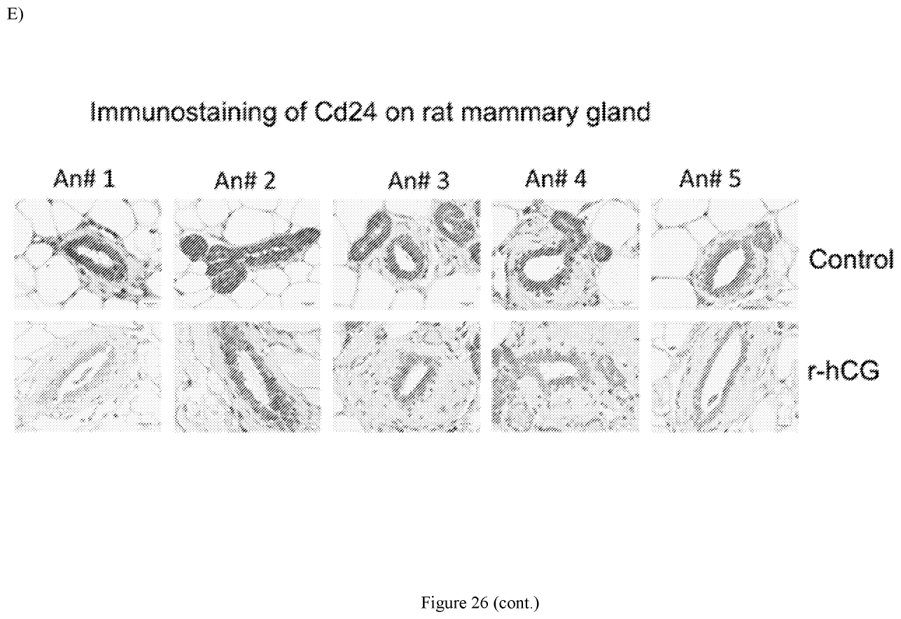

The finding in clinical trial that r-hCG treatment inhibited the expression of genes related to stem cell proliferation and maintenance is consistent with the data from an animal study on investigating the effect of r-hCG on sternness of rat mammary epithelial cells. The number of primary mammospheres formed by mammary epithelial cells of r-hCG treated rats was significantly reduced compared with that of control rats (FIG. 26, Panel A), suggesting the stemness of mammary stem cells/progenitors was suppressed. Microarray was performed using RNA extracted from primary mammospheres. There were 223 (117 up, 106 down; FC 1.5) and 95 DEGs (48 up, 47 down; FC 2.0) with FDR p<0.05 (FIG. 26, Panel B). GO analysis showed the top GO of up and down-regulated genes was system development and biological regulation, respectively (FIG. 26, Panel C). Canonical pathway analysis revealed the top pathway enriched by up-regulated genes was Wnt/β-catenin signaling, while pathways enriched by down-regulated genes were immune-related pathways (FIG. 26, Panel D). It is important to note that the genes up-regulated in Wnt/β-catenin signaling including SOX7, SOX17, SOX18, and SFRP4 are negative regulators of Wnt/β-catenin signaling, whereas Wnt ligand WNT2 was down-regulated by r-hCG, suggesting the inhibition of the Wnt signaling (data not shown).

Focusing on the analysis of stem cell/progenitor markers, it was demonstrated that Cd24 and MIE were significantly reduced in mammospheres derived from r-hCG treated rats (Russo et al., The Role of Stem Cell in Breast Cancer Prevention; In: Russo J, Russo I H, editors, Role of the Transcriptome in Breast Cancer Prevention, New York, Springer US, 2013, 403-439). It was further confirmed that Cd24 expression was significantly decreased in the mammary gland ducts of r-hCG treated rats (FIG. 26, Panel E). Taken together, the data suggest that r-hCG suppresses the stemness of mammary epithelial cells, might in part mediated by inhibiting Wnt/β-catenin signaling.

### Example 12: R-hCG Treatment Upregulates BRCA1, BARD1, FOXO3, and p53, Promotes DNA Repair, and Induces Chromatin Remodeling in Breast Epithelial Cells In Vitro

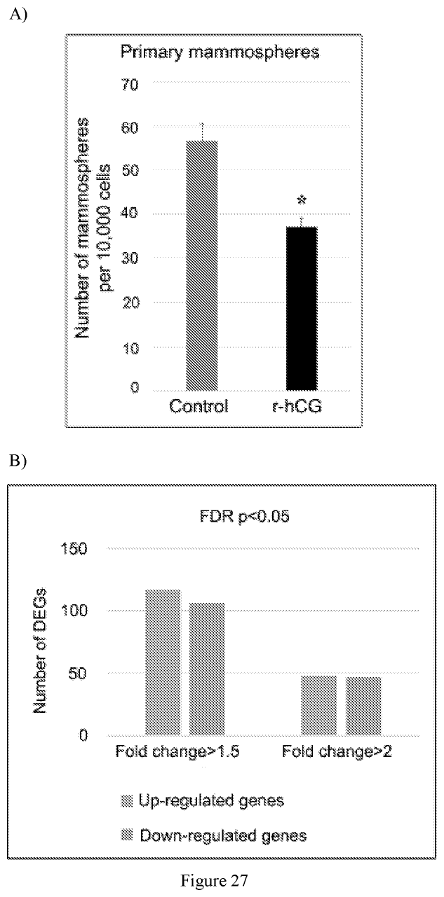

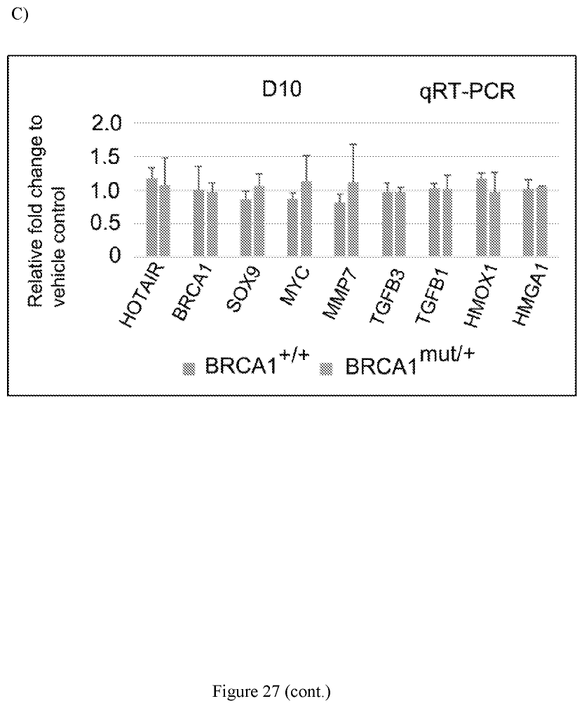

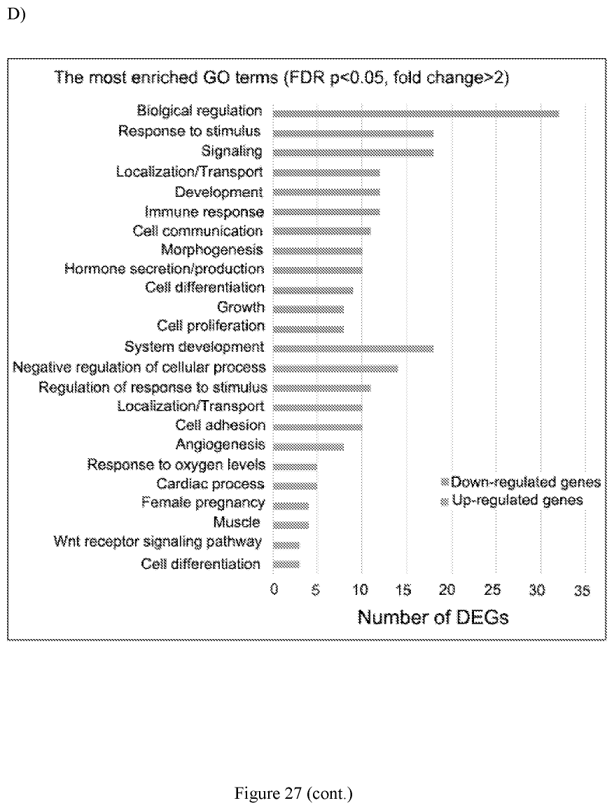

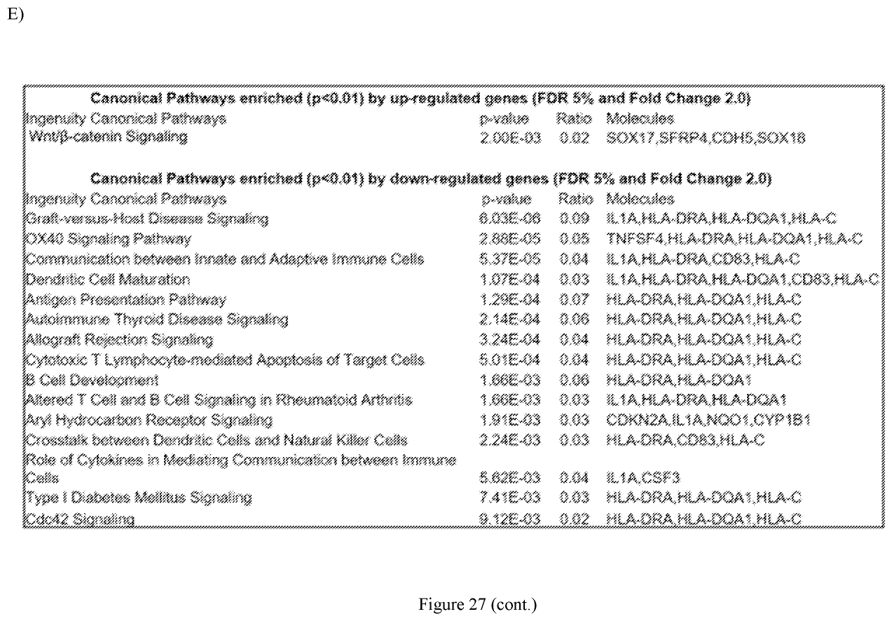

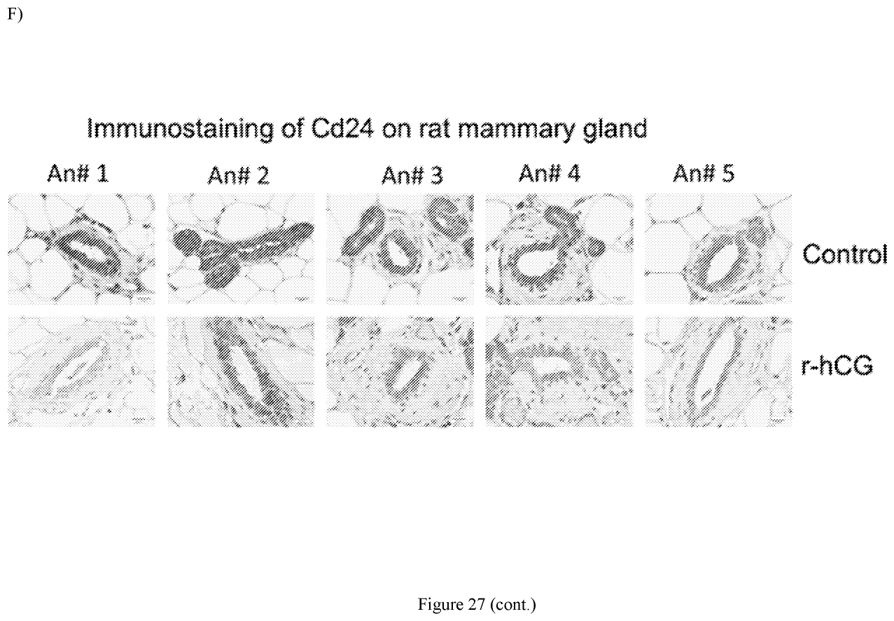

MCF10A human breast epithelial cells with engineered BRCA1 haploinsufficiency (BRCA1^{mut/+}) and its isogenic parental BRCA1^{+/+} cells was purchased from Horizon Discovery, and treated cells with r-hCG in vitro (FIG. 27, Panel A). RNA expression was evaluated for some genes that showed expression changes in BRCA1/2 carriers in the hCG clinical trial. There was a 1.29-fold increase in TGFB3 expression at the end of 3-day r-hCG treatment (DO time point) in BRCA1^{+/+} cells, and a significant decrease of miR182 in the two cell lines after r-hCG treatment (FIG. 27, Panel B and Panel C). Interestingly, it was identified that SOX9, HOTAIR, and MYC expression were higher in BRCA1^{mut/+} compared to BRCA1^{+/+} cells, indicating the up-regulation of genes related to stem cell maintenance and cell transformation in BRCA1 haploinsufficient cells (FIG. 27, Panel D).

The protein expression of BRCA1 and FOXO3, two targets of miR182, was evaluated. Consistently, a significant increase in full-length BRCA1 protein and FOXO3 after r-hCG treatment was observed. BARD1, the major BRCA1 partner, was increased in a pattern similar to that of BRCA1 (FIG. 27, Panel E). P53 was also increased at D6 and/or D10. In addition, (3-casein protein expression was increased at D6 in both cell lines, suggesting the induction of cell differentiation (data not shown).

The expression of some key components in Wnt and TGFβ signaling was examined. Consistently, increase of SOX7, SOX17, SFRP4, and TGFβ protein expression were observed at different time points upon r-hCG treatment (data not shown), suggesting the inhibition of Wnt and activation of TGFβ signaling.

The observation that r-hCG regulates BRCA1, BARD1, and p53 led us to examine its effect on DNA repair. It was demonstrated that γ-H2AX level was significantly reduced in r-hCG treated cells evaluated 6 and 24 hours after gamma irradiation compared to control cells (FIG. 27, Panel F), indicating that r-hCG treated cells repair DNA faster and better.

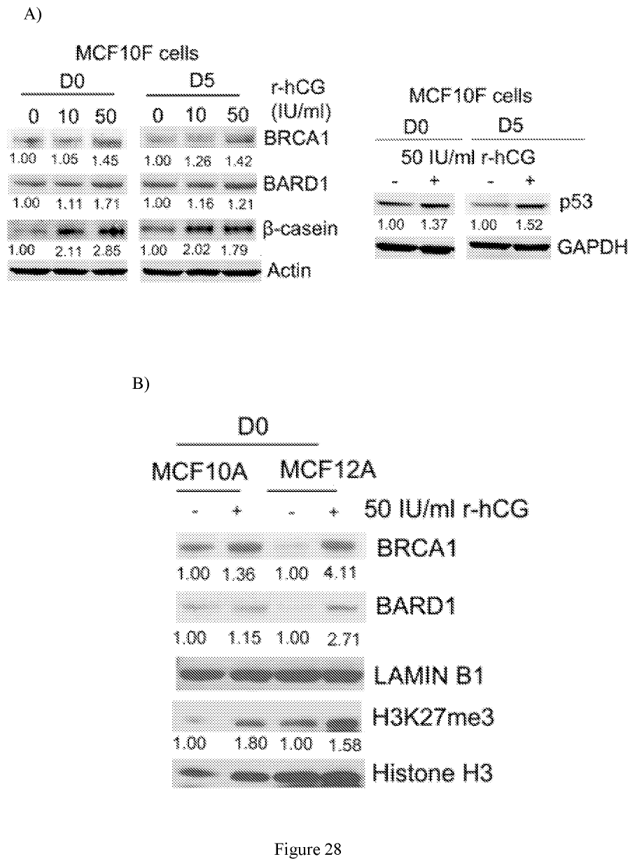

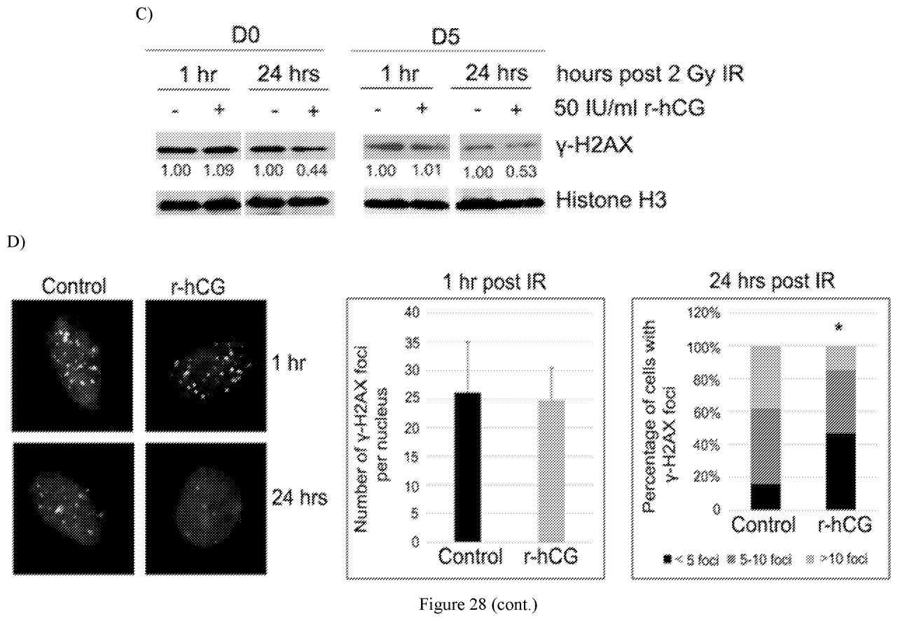

Furthermore, it was demonstrated that the chromatin remodeling marker H3K27me3 was increased in r-hCG treated cells (data not shown). The study was extended to two other human breast epithelial cell lines MCF10F and MCF12A and confirmed the up-regulation of BRCA1, BARD1, β-casein, and H3K27me3 and the increase of DNA repair capacity by r-hCG (FIG. 28, Panel A, Panel B, Panel C, and Panel D).

Altogether, these results indicate that r-hCG has a direct role in inducing full-length BRCA1, BARD1, FOXO3, and p53 expression in breast epithelial cells, might partially through Wnt signaling inhibition and TGFβ activation. The up-regulation of these proteins is more prominent after the cessation of treatment, suggesting the involvement of epigenetic mechanism. Furthermore, r-hCG promotes DNA repair in cultured cells. These data confirm the findings from the hCG clinical trial and suggest that r-hCG plays an important role in cell differentiation, DNA repair, and chromatin remodeling in breast epithelial cells.

### Discussion:

Based on the data from this study, the following was concluded. First, r-hCG treatment induces significant gene expression changes in the breast tissue of BRCA1/2 carriers; these genes are mainly related to development, cell differentiation, cell cycle, apoptosis, stem cell proliferation, DNA repair, chromatin organization and remodeling, and GPCR signaling. Second, r-hCG inhibits Wnt signaling and suppresses stemness of breast/mammary epithelial cells. Third, r-hCG activates TGFB/TGFBR-SMAD2/3/4, TP53, and BRCA1, whereas inhibits MYC in the breast tissue of BRCA1/2 carriers. Fourth, r-hCG directly upregulates tumor suppressor proteins BRCA1, BARD1, FOXO3, and p53 expression, induces chromatin remodeling and cell differentiation, and promotes DNA repair in cultured breast epithelial cells. Fifth, r-hCG inhibits the expression of non-coding RNA HOTAIR and miR182 in the breast tissue of BRCA1/2 carriers and/or cultured breast epithelial cells. In addition, a clear difference was observed in the response to r-hCG treatment, the serum progesterone level (Depypere et al., Eur. J. Cancer Prev., 2021, 30, 195-203), and changes in GPCR signaling between the two groups (with or without hormonal contraceptives).

In this study, it was observed that a great number of r-hCG up-regulated genes are involved in cell development and differentiation, suggesting r-hCG can induce breast development in BRCA1/2 carriers.

The present study also showed that r-hCG treatment inhibits WNT/β-catenin signaling in the breast of BRCA carriers. Numerous negative regulators of WNT signaling including SOX7, SOX17, SOX18, SFRP2, SFRP4, DKK3, and LRP1, were up-regulated by r-hCG, whereas positive regulators FZD1, FZD7, SOX9, SOX10, and WNT signaling target genes MMP7 and MYC, as well as MYC target gene HOTAIR were down-regulated. Of importance, SOX9, SOX10, FZD7, and MYC are implicated in maintaining human breast luminal progenitor and cancer stem cells (Domenici et al., Oncogene, 2019, 38, 3151-3169; Moumen et al., Mol. Cancer., 2013, 12, 132; and Chakrabarti et al., Nat. Cell Biol., 2014, 16, 1004-1015, 1-13), and the expression of SOX9, SOX10, FZD7, HOTAIR, MYC, and MMP7 are all positively correlated with triple negative status of the breast cancer (Wang et al., Minerva Med., 2017, 108, 513-517; and Wang et al., Oncotarget, 2015, 6, 11150-11161). It was further revealed that r-hCG treatment inhibits Wnt/β-catenin signaling in rat mammospheres and cultured breast epithelial cells. Altogether, these findings suggest that r-hCG treatment may protect BRCA1/2 carriers from breast cancer partially by suppressing stemness of breast epithelial cells mediated by Wnt signaling inhibition. In addition, the effect of hCG on Wnt signaling may also contribute to pregnancy-induced breast cancer prevention.

Another important finding of this study is the activation of upstream regulators TP53, TGFB/TGFBR-SMAD2/3/4, and BRCA1 by r-hCG. Interestingly, in this study, TGFB1/2 and TGFBR1/2 were predicted activated by r-hCG. TGFB1 and TGFB3 RNA levels were increased and numerous TGFβ signaling target genes including ID4 were altered. ID4 was down-regulated by r-hCG in this study. miR182 was down-regulated whereas BRCA1 protein was up-regulated and activated as an upstream regulator, suggesting that r-hCG may be used as a hormonal regulator to rescue BRCA1 haploinsufficiency for BRCA1 carriers. It was also demonstrated that Kit, one important BRCA1 target gene, was down-regulated by r-hCG. Consistently, MYC activity was predicted inhibited in this study. Collectively, these results strongly suggest that r-hCG treatment leads to activation of TP53, TGFB/TGFBR-SMAD and BRCA1, whereas inhibition of MYC, events that are crucial for breast epithelial differentiation and lineage commitment, DNA repair, and prevention of neoplastic transformation.

Epigenetics offers new horizons for cancer prevention. In this study, it was observed that r-hCG induced a long-lasting change in gene expression. Epigenetic mechanisms may be involved in this change. One important finding is the inhibition of chromatin remodeling gene HMGA1 by r-hCG. Expression changes were also observed in HOTAIR, miR182 and H3K27me3 after r-hCG treatment. These findings suggest that r-hCG may be used as an epigenetic modulator for breast cancer prevention.

Administration of r-hCG affects genes/signaling pathways controlling stem/progenitor cell maintenance and differentiation, mammary epithelial cell commitment, genomic stability, neoplastic transformation, and other biological processes in the breast of BRCA1/2 carriers, and may subsequently lead to reduce the risk to breast cancer. Furthermore, the protective effects of r-hCG might expand beyond breast cancer since BRCA1/2 carriers are also at high risk for ovarian cancer, and also expand to other women at risk for breast cancer or to the general population.

In conclusion, the findings herein indicate that in the breast of BRCA1/2 carriers, BRCA1/2 mutation affects not only genome stability, but also pathways related to breast progenitor cell maintaining, cell differentiation, and neoplastic transformation. Experimental evidence provided in this study indicate that these pathways can be modified by r-hCG treatment. Most importantly, Wnt signaling and MYC, the two pathways that lead to neoplastic transformation and tumorigenesis, are inhibited by r-hCG. The data highlight that r-hCG may be used as a preventative agent against breast cancer for BRCA1/2 carriers.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of treating a nulligravid female having a high risk of developing breast cancer, the method comprising administering human chorionic gonadotropin (hCG) two to four times a week for at least ten weeks, wherein the nulligravid female is without exposure to a contraceptive for at least 21 days prior to administration of the hCG, thereby reducing the risk of developing breast cancer.

2. The method of claim 1, wherein the hCG is administered: two to four times a week for at least eleven weeks; two to four times a week for at least twelve weeks; two to four times a week for no more than twelve weeks; three times a week for at least eleven weeks; three times a week for at least twelve weeks; or three times a week for no more than twelve weeks.

3. The method of claim 1 or claim 2, wherein the nulligravid female is without exposure to a contraceptive for at least 26 days prior to administration of the hCG.

4. The method of any one of claims 1 to 3, wherein the nulligravid female is without exposure to a contraceptive for at least 30 days prior to administration of the hCG.

5. The method of any one of claims 1 to 4, wherein the contraceptive is an oral hormonal contraceptive, a transdermal contraceptive, or an implanted contraceptive.

6. The method of claim 5, wherein the implanted contraceptive is levonorgestrel (LNG) intrauterine device (IUD), LNG-releasing intrauterine system (LNG-IUS), or a progestin IUD.

7. The method of any one of claims 1 to 6, wherein the nulligravid female is a carrier of a deleterious mutation in any one or more of BRCA1, BRCA2, PALP2, CHEK2, ATM, TP53, RAD51C, RAD51d, BRIP1, MLH1, MSH2, and MSH6.

8. The method of claim 7, wherein the nulligravid female is a carrier of a deleterious mutation in BRCA1 and/or BRCA2.

9. The method of any one of claims 1 to 8, wherein the nulligravid female is: from about 18 years of age to about 40 years of age; from about 18 years of age to about 30 years of age; from about 18 years of age to about 26 years of age; or from about 19 years of age to about 29 years of age.

10. The method of any one of claims 1 to 9, wherein the hCG is administered to the nulligravid female during the luteal phase.

11. The method of any one of claims 1 to 10, wherein the hCG is administered in an amount: from about 50 μg to about 500 μg; from about 100 μg to about 400 μg; from about 200 μg to about 300 μg; or about 250 μg.

12. The method of any one of claims 1 to 11, wherein the hCG is administered subcutaneously, transdermally, intranasally, by an intravaginal ring or implant, or by a controlled release device.

13. The method of claim 12, wherein the hCG is administered by subcutaneous injection.

14. The method of claim 12, wherein the hCG is administered as a slow release formulation by an implanted controlled release device.

15. The method of any one of claims 1 to 14, wherein the hCG is recombinant hCG (rhCG) or urinary hCG, or any therapeutically active peptide thereof.

16. The method of claim 15, wherein the hCG peptide comprises the amino acid sequence Ala Leu Cys Arg Arg Ser Thr Thr Asp Cys Gly Gly Pro Lys Asp His Pro Leu Thr Ser (SEQ ID NO:1), Ser Tyr Ala Val Ala Leu Ser Cys Gln Cys Ala Leu Cys Arg Arg (SEQ ID NO:2), Ser Leu Glu Pro Leu Arg Pro Arg Cys Arg Pro Ile Asn Ala Thr (SEQ ID NO:3), Ser Tyr Ala Val Ala Leu Ser Ala Gln Cys Ala Leu Cys Arg Arg (SEQ ID NO:4), or Ser Phe Pro Val Ala Leu Ser Cys Arg Cys Gly Pro Cys Arg Arg (SEQ ID NO:5).

17. The method of claim 15, wherein the hCG is rhCG.

18. A method of monitoring the efficacy of treatment of a subject having breast cancer or having a high risk of developing breast cancer, the method comprising:
a) obtaining or having obtained a biological sample from the subject prior to treatment initiation (T1) to provide a baseline expression of a panel of genes from the biological sample;
b) obtaining or having obtained a biological sample from the subject after treatment completion (T2);
c) obtaining or having obtained a biological sample from the subject about 6 months or later after treatment completion (T3); and
d) performing a gene expression assay on the T1, T2, and T3 samples to identify a set of differentially expressed genes from the biological sample;
wherein increased expression in at least 10 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 5 of the following genes: FBL, FZD1, FZD7, HMAG1, ITGB4, LIG1, KIT, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample at T3 compared to T1; indicates that the treatment is efficacious, and wherein a lack of the differentially expressed gene profile indicates that the treatment is non-efficacious.

19. The method of claim 18, wherein the biological sample is breast tissue, blood, or urine, or any combination thereof.

20. The method of claim 18 or claim 19, wherein the biological sample for identification of the baseline expression of the panel of genes is obtained from the subject about 3 months prior to treatment initiation.

21. The method of any one of claims 18 to 20, wherein the biological sample obtained from the subject after treatment completion in step b) is obtained from the subject from about 1 day to about 7 days after treatment completion.

22. The method of claim 21, wherein the biological sample obtained from the subject after treatment completion in step b) is obtained from the subject within 3 days after treatment completion.

23. The method of claim 21, wherein the biological sample obtained from the subject after treatment completion in step b) is obtained from the subject within one or two days after treatment completion.

24. The method of any one of claims 18 to 23, wherein the treatment comprises administering hCG to the subject.

25. The method of claim 24, wherein the hCG is recombinant hCG (rhCG) or urinary hCG, or any therapeutically active peptide thereof.

26. The method of claim 25, wherein the hCG peptide comprises the amino acid sequence Ala Leu Cys Arg Arg Ser Thr Thr Asp Cys Gly Gly Pro Lys Asp His Pro Leu Thr Ser (SEQ ID NO:1), Ser Tyr Ala Val Ala Leu Ser Cys Gln Cys Ala Leu Cys Arg Arg (SEQ ID NO:2), Ser Leu Glu Pro Leu Arg Pro Arg Cys Arg Pro Ile Asn Ala Thr (SEQ ID NO:3), Ser Tyr Ala Val Ala Leu Ser Ala Gln Cys Ala Leu Cys Arg Arg (SEQ ID NO:4), or Ser Phe Pro Val Ala Leu Ser Cys Arg Cys Gly Pro Cys Arg Arg (SEQ ID NO:5).

27. The method of claim 24, wherein the hCG is rhCG.

28. The method of any one of claims 18 to 27, wherein increased expression in at least 20 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 8 of the following genes: FBL, FZD1, FZD7, HMAG1, ITGB4, LIG1, KIT, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 20 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 5 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and wherein a lack of the differentially expressed gene profile indicates that the treatment is non-efficacious.

29. The method of any one of claims 18 to 27, wherein increased expression in at least 30 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 10 of the following genes: FBL, FZD1, FZD7, HMAG1, ITGB4, LIG1, KIT, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 30 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 6 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and wherein a lack of the differentially expressed gene profile indicates that the treatment is non-efficacious.

30. The method of any one of claims 18 to 27, wherein increased expression in at least 40 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 12 of the following genes: FBL, FZD1, FZD7, HMAG1, ITGB4, LIG1, KIT, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 40 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 7 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and wherein a lack of the differentially expressed gene profile indicates that the treatment is non-efficacious.

31. The method of any one of claims 18 to 27, wherein increased expression in at least 50 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 15 of the following genes: FBL, FZD1, FZD7, HMAG1, ITGB4, LIG1, KIT, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 50 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 8 of the following genes: EYA2, FZD1, HMGA1, ID4, KIT, miR182, MMP7, MYC, PADI2, SOX9, and SOX10, in the biological sample at T3 compared to T1 indicates that the treatment is efficacious, and wherein a lack of the differentially expressed gene profile indicates that the treatment is non-efficacious.

32. The method of any one of claims 18 to 31, wherein upon an indication of efficacious treatment, the treatment can be discontinued.

33. The method of any one of claims 18 to 31, wherein upon an indication of non-efficacious treatment, the treatment can be altered to a different treatment.

34. The method of any one of claims 18 to 31, wherein the subject is a carrier of a deleterious mutation in any one or more of BRCA1, BRCA2, PALP2, CHEK2, ATM, TP53, RAD51C, RAD51d, BRIP1, MLH1, MSH2, and MSH6.

35. The method of claim 34, wherein the subject is a carrier of a deleterious mutation in BRCA1 and/or BRCA2.

36. The method of any one of claims 18 to 35, wherein the subject having breast cancer or having a high risk of developing breast cancer is a nulligravid female without exposure to a contraceptive for at least 26 days prior to treatment initiation (T1).

37. The method of claim 36, wherein the subject is without exposure to a contraceptive for at least 30 days prior to treatment initiation (T1).

38. The method of claim 36, wherein the contraceptive is an oral hormonal contraceptive, a transdermal contraceptive, or an implanted contraceptive.

39. The method of claim 38, wherein the implanted contraceptive is levonorgestrel (LNG) intrauterine device (IUD), LNG-releasing intrauterine system (LNG-IUS), or a progestin IUD.

40. The method of any one of claims 18 to 39, wherein the subject is a female: from about 18 years of age to about 40 years of age; from about 18 years of age to about 30 years of age; from about 18 years of age to about 26 years of age; or from about 19 years of age to about 29 years of age.

41. A method of monitoring the efficacy of treatment of a subject having breast cancer or having a high risk of developing breast cancer, the method comprising:
a) obtaining or having obtained a biological sample from the subject prior to treatment initiation (T1) to provide a baseline expression of a panel of genes from the biological sample; and
b) obtaining or having obtained a biological sample from the subject after treatment initiation (T1); and
c) performing a gene expression assay on the two samples to identify a set of differentially expressed genes from the biological sample;
wherein increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

42. The method of claim 41, wherein the biological sample is breast tissue, blood, or urine, or any combination thereof.

43. The method of claim 41 or claim 42, wherein the biological sample for identification of the baseline expression of the panel of genes is obtained from the subject about 3 months prior to treatment initiation.

44. The method of any one of claims 41 to 43, wherein the biological sample obtained from the subject after treatment initiation in step b) is obtained from the subject from about 1 month to about 9 months after treatment initiation.

45. The method of claim 44, wherein the biological sample obtained from the subject after treatment initiation in step b) is obtained from the subject from about 3 months to about 9 months after treatment initiation.

46. The method of claim 44, wherein the biological sample obtained from the subject after treatment initiation in step b) is obtained from the subject from about 6 months to about 9 months after treatment initiation.

47. The method of any one of claims 41 to 46, wherein the treatment comprises administering hCG to the subject.

48. The method of claim 47, wherein the hCG is recombinant hCG (rhCG) or urinary hCG, or any therapeutically active peptide thereof.

49. The method of claim 48, wherein the hCG peptide comprises the amino acid sequence Ala Leu Cys Arg Arg Ser Thr Thr Asp Cys Gly Gly Pro Lys Asp His Pro Leu Thr Ser (SEQ ID NO:1), Ser Tyr Ala Val Ala Leu Ser Cys Gln Cys Ala Leu Cys Arg Arg (SEQ ID NO:2), Ser Leu Glu Pro Leu Arg Pro Arg Cys Arg Pro Ile Asn Ala Thr (SEQ ID NO:3), Ser Tyr Ala Val Ala Leu Ser Ala Gln Cys Ala Leu Cys Arg Arg (SEQ ID NO:4), or Ser Phe Pro Val Ala Leu Ser Cys Arg Cys Gly Pro Cys Arg Arg (SEQ ID NO:5).

50. The method of claim 47, wherein the hCG is rhCG.

51. The method of any one of claims 41 to 50, wherein increased expression in at least 20 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 5 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

52. The method of any one of claims 41 to 50, wherein increased expression in at least 30 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 6 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

53. The method of any one of claims 41 to 50, wherein increased expression in at least 40 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 7 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

54. The method of any one of claims 41 to 50, wherein increased expression in at least 50 of the following genes: ADAMTSL4, BMP1, BMP6, BRCA1, CASP10, CBX2, CCN2, CD28, CREB3L1, DAB2, EPB4IL3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 8 of the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

55. The method of any one of claims 41 to 54, wherein upon an indication of efficacious treatment, the treatment can be discontinued.

56. The method of any one of claims 41 to 54, wherein upon an indication of non-efficacious treatment, the treatment can be continued or altered to a different treatment.

57. The method of any one of claims 41 to 56, wherein the subject is a carrier of a deleterious mutation in any one or more of BRCA1, BRCA2, PALP2, CHEK2, ATM, TP53, RAD51C, RAD51d, BRIP1, MLH1, MSH2, and MSH6.

58. The method of claim 57, wherein the subject is a carrier of a deleterious mutation in BRCA1 and/or BRCA2.

59. The method of any one of claims 41 to 58, wherein the subject having breast cancer or having a high risk of developing breast cancer is a nulligravid female without exposure to a contraceptive for at least 26 days prior to treatment initiation (T1).

60. The method of claim 59, wherein the subject is without exposure to a contraceptive for at least 30 days prior to treatment initiation (T1).

61. The method of claim 59, wherein the contraceptive is an oral hormonal contraceptive, a transdermal contraceptive, or an implanted contraceptive.

62. The method of claim 61, wherein the implanted contraceptive is levonorgestrel (LNG) intrauterine device (IUD), LNG-releasing intrauterine system (LNG-IUS), or a progestin IUD.

63. The method of any one of claims 41 to 62, wherein the subject is a female: from about 18 years of age to about 40 years of age; from about 18 years of age to about 30 years of age; from about 18 years of age to about 26 years of age; or from about 19 years of age to about 29 years of age.

64. A method of determining whether a subject is at risk of developing breast cancer, the method comprising obtaining or having obtained a biological sample from the subject and performing a gene expression assay to identify an expression profile of a panel of genes from the biological sample;
wherein increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer; and when the subject does not have the expression profile, the subject is at higher risk of developing breast cancer.

65. The method of claim 64, wherein increased expression in at least 20 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 5 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer; and when the subject does not have the expression profile, the subject is at higher risk of developing breast cancer.

66. The method of claim 64, wherein increased expression in at least 30 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 6 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer; and when the subject does not have the expression profile, the subject is at higher risk of developing breast cancer.

67. The method of claim 64, wherein increased expression in at least 40 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 7 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer; and when the subject does not have the expression profile, the subject is at higher risk of developing breast cancer.

68. The method of claim 64, wherein increased expression in at least 50 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 8 of the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer; and when the subject does not have the expression profile, the subject is at higher risk of developing breast cancer.

69. The method of any one of claims 64 to 68, wherein the control breast cancer expression profile is derived from a subject having breast cancer.

70. The method of any one of claims 64 to 69, wherein the biological sample is breast tissue, blood, or urine, or any combination thereof.

71. The method of any one of claims 64 to 70, wherein the subject is a female: from about 18 years of age to about 40 years of age; from about 18 years of age to about 30 years of age; from about 18 years of age to about 26 years of age; or from about 19 years of age to about 29 years of age.

72. The method of any one of claims 64 to 71, wherein when the subject does not have the expression profile, the subject is further treated to prevent the development of breast cancer.

73. The method of claim 72, wherein the subject is a carrier of a deleterious mutation in any one or more of BRCA1, BRCA2, PALP2, CHEK2, ATM, TP53, RAD51C, RAD51d, BRIP1, MLH1, MSH2, and MSH6.

74. The method of claim 73, wherein the subject is a carrier of a deleterious mutation in BRCA1 and/or BRCA2.

75. The method of any one of claims 72 to 74, wherein the treatment comprises administering hCG, or a therapeutically active peptide thereof, to the subject.

76. The method of claim 75, wherein the hCG is recombinant hCG (rhCG) or urinary hCG, or any therapeutically active peptide thereof.

77. The method of claim 76, wherein the hCG peptide comprises the amino acid sequence Ala Leu Cys Arg Arg Ser Thr Thr Asp Cys Gly Gly Pro Lys Asp His Pro Leu Thr Ser (SEQ ID NO:1), Ser Tyr Ala Val Ala Leu Ser Cys Gln Cys Ala Leu Cys Arg Arg (SEQ ID NO:2), Ser Leu Glu Pro Leu Arg Pro Arg Cys Arg Pro Ile Asn Ala Thr (SEQ ID NO:3), Ser Tyr Ala Val Ala Leu Ser Ala Gln Cys Ala Leu Cys Arg Arg (SEQ ID NO:4), or Ser Phe Pro Val Ala Leu Ser Cys Arg Cys Gly Pro Cys Arg Arg (SEQ ID NO:5).

78. The method of claim 76, wherein the hCG is rhCG.

79. The method of any one of claims 72 to 78, wherein the treatment comprises administering from about 50 μg to about 500 μg of hCG two to four times a week for at least ten weeks.

80. The method of claim 79, wherein the hCG is administered: two to four times a week for at least eleven weeks; two to four times a week for at least twelve weeks; two to four times a week for no more than twelve weeks; three times a week for at least eleven weeks; three times a week for at least twelve weeks; or three times a week for no more than twelve weeks.

81. The method of claim 79 or claim 80, wherein the hCG is administered in an amount: from about 100 μg to about 400 μg; from about 200 μg to about 300 μg; or about 250 μg.

82. The method of any one of claims 72 to 81, wherein the subject is: a nulligravid female without exposure to a contraceptive for at least 21 days prior to administration of the hCG; a nulligravid female without exposure to a contraceptive for at least 26 days prior to administration of the hCG; or a nulligravid female without exposure to a contraceptive for at least 30 days prior to administration of the hCG.

83. The method of claim 82, wherein the contraceptive is an oral hormonal contraceptive, a transdermal contraceptive, or an implanted contraceptive.

84. The method of claim 83, wherein the implanted contraceptive is levonorgestrel (LNG) intrauterine device (IUD), LNG-releasing intrauterine system (LNG-IUS), or a progestin IUD.

85. Human chorionic gonadotropin (hCG), or a therapeutically active peptide thereof, for use in treating a nulligravid female having a high risk of developing breast cancer, the treating comprising administering hCG, or a therapeutically active peptide thereof, two to four times a week for at least ten weeks, wherein the nulligravid female is without exposure to a contraceptive for at least 21 days prior to administration of the hCG.

86. The hCG, or therapeutically active peptide thereof, of claim 85, wherein the contraceptive is an oral hormonal contraceptive, a transdermal contraceptive, or an implanted contraceptive.

87. The hCG, or therapeutically active peptide thereof, of claim 85 or claim 86, wherein the nulligravid female is a carrier of a deleterious mutation in a gene selected from the group consisting of: BRCA1, BRCA2, PALP2, CHEK2, ATM, TP53, RAD51C, RAD51d, BRIP1, MLH1, MSH2, and MSH6.

88. The hCG, or therapeutically active peptide thereof, of any one of claims 85 to 87, wherein the nulligravid female is from about 18 years of age to about 40 years of age.

89. The hCG, or therapeutically active peptide thereof, of any one of claims 85 to 88, wherein, the hCG is recombinant hCG (rhCG) or urinary hCG, or wherein the therapeutically active peptide comprises the amino acid sequence Ala Leu Cys Arg Arg Ser Thr Thr Asp Cys Gly Gly Pro Lys Asp His Pro Leu Thr Ser (SEQ ID NO:1), Ser Tyr Ala Val Ala Leu Ser Cys Gln Cys Ala Leu Cys Arg Arg (SEQ ID NO:2), Ser Leu Glu Pro Leu Arg Pro Arg Cys Arg Pro Ile Asn Ala Thr (SEQ ID NO:3), Ser Tyr Ala Val Ala Leu Ser Ala Gln Cys Ala Leu Cys Arg Arg (SEQ ID NO:4), or Ser Phe Pro Val Ala Leu Ser Cys Arg Cys Gly Pro Cys Arg Arg (SEQ ID NO:5).

90. The hCG of any one of claims 85 to 89, wherein the hCG is administered in an amount from about 50 μg to about 500 μg.

91. A method of monitoring the efficacy of treatment of a subject having breast cancer or having a high risk of developing breast cancer, the method comprising:
a) obtaining or having obtained a biological sample from the subject prior to treatment initiation (T1) to provide a baseline expression of a panel of genes from the biological sample;
b) obtaining or having obtained a biological sample from the subject after treatment completion (T2);
c) obtaining or having obtained a biological sample from the subject about 6 months or later after treatment completion (T3); and
d) performing a gene expression assay on the T1, T2, and T3 samples to identify a set of differentially expressed genes from the biological sample;
wherein increased expression in at least 10 of the following genes: ADAMTSL4, ANXA2, ASPN, BIN, BIVM-ERCC5, BMP1, BRCA1, CAV1, CAV2, CCDC80, CCN2, DKK3, ELN, ETS, FBLN1, FBLN2, FN1, FOXO3, FZD4, GAS1, GATA2, GPER1, HIC1, HMOX1, HSPB1, IGFBP3, ISG15, MMP2, MYCT1, NQO1, PADI3, PMEPA1, PRKCD, RECQL, SAMHD1, SATB2, SFRP2, SFRP4, SOX7, SOX17, SOX18, TIMP1, TIMP3, TGFB1, TGFB3, and TGFBR2, and decreased expression in at least 5 of the following genes: FBL, FZD1, FZD7, HMAG1, ITGB4, LIG1, KIT, miR182, NPM2, MMP7, MYC, MYCL, PADI2, PROM1, RPS6, RPS12, RPS18, RPS19, SOX9, and SOX10, in the biological sample at T2 compared to T1; and/or increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample at T3 compared to T1; indicates that the treatment is efficacious, and wherein a lack of the differentially expressed gene profile indicates that the treatment is non-efficacious.

92. The method of claim 91, wherein the biological sample obtained from the subject after treatment completion in step b) is obtained from the subject: i) from about 1 day to about 7 days after treatment completion; ii) within 3 days after treatment completion; or iii) within one or two days after treatment completion.

93. The method of claim 91 or claim 92, wherein the subject having breast cancer or having a high risk of developing breast cancer is a nulligravid female without exposure to a contraceptive for at least 26 days prior to treatment initiation (T1).

94. A method of monitoring the efficacy of treatment of a subject having breast cancer or having a high risk of developing breast cancer, the method comprising:
a) obtaining or having obtained a biological sample from the subject prior to treatment initiation (T1) to provide a baseline expression of a panel of genes from the biological sample; and
b) obtaining or having obtained a biological sample from the subject after treatment initiation (T1); and
c) performing a gene expression assay on the two samples to identify a set of differentially expressed genes from the biological sample;
wherein increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample obtained in step b) compared to step a) indicates that the treatment is efficacious, and wherein a lack of the differentially expressed gene profile indicates that the treatment is not yet efficacious.

95. The method of any one of claims 91 to 94, wherein the biological sample is breast tissue, blood, or urine, or any combination thereof.

96. The method of any one of claims 91 to 95, wherein the biological sample for identification of the baseline expression of the panel of genes is obtained from the subject about 3 months prior to treatment initiation.

97. The method of any one of claims 94 to 96, wherein the biological sample obtained from the subject after treatment initiation in step b) is obtained from the subject: i) from about 1 month to about 9 months after treatment initiation; ii) from about 3 months to about 9 months after treatment initiation; or iii) from about 6 months to about 9 months after treatment initiation.

98. A method of determining whether a subject is at risk of developing breast cancer, the method comprising obtaining or having obtained a biological sample from the subject and performing a gene expression assay to identify an expression profile of a panel of genes from the biological sample;
wherein increased expression in at least 10 of the following genes: ADAMTSL4, BMP1, BMP6, BRCA1, CASP10, CBX2, CCN2, CD28, CREB3L1, DAB2, EPB41L3, FBLN2, FN1, FOXO3, FZD4, GAS1, GDF10, GIMAP8, GPX3, HIC1, HMOX1, HSPB1, ID3, IGFBP3, INHBA, KLF4, LATS2, MEF2C, MIX1, MMP2, MYCT1, NQO1, OSR1, PLAGL1, PRUNE2, PTGIS, ROBO2, RPS6KA2, SAMHD1, SAT1, SFRP2, SFRP4, SILF1, SLIT2, SLIT3, SOX17, SOX18, SOX7, TIMP1, TGFB1, TGFB3, TGFBR2, and TMEM173, and decreased expression in at least 4 the following genes: EYA2, FZD1, HMGA1, ID4, KIT, miR182, MMP7, MYC, PADI2, SOX9, and SOX10, in the biological sample obtained from the subject compared to a control breast cancer expression profile indicates that the subject has a lower risk of developing breast cancer, and when the subject does not have the expression profile, the subject is at higher risk of developing breast cancer.

99. The method of claim 98, wherein the biological sample is breast tissue, blood, or urine, or any combination thereof, and the subject is a nulligravid female without exposure to a contraceptive for at least 21 days prior to administration of the hCG.
